(19) United States
(12) Patent Application Publication
(10) Pub. No.: US 2025/0008959 A1
(43) Pub. Date: Jan. 9, 2025

(54) HERBICIDES AND USE THEREOF

(71) Applicant: Projini Agchem LTD, D.N. Upper Galil (IL)

(72) Inventors: Nesly DOTAN, Kibbutz HaGoshrim (IL); Itai BLOCH, Rehovot (IL); Maayan GAL, Givat-Ada (IL); Elad COHEN, Kiryat-Shmona (IL); Rotem BEN-SHUSHAN SHELLY, Kibbutz Kfar-Blum (IL); Eytan AMRAM, Kibbutz Iftach (IL)

(73) Assignee: Projini Agchem LTD, D.N. Upper Galil (IL)

(21) Appl. No.: 18/699,617

(22) PCT Filed: Oct. 10, 2022
(86) PCT No.: PCT/IL2022/051075

§ 371 Date: Apr. 9, 2024

Related U.S. Application Data
(60) Provisional application No. 63/254,193, filed on Oct. 11, 2021.

(51) Int. Cl. A01N 43/78; A01N 43/08; A01N 43/54; A01N 43/84; A01P 13/00; C07C 237/40; C07D 233/96; C07D 265/30; C07D 277/46; C07D 307/68

(52) U.S. Cl. CPC A01N 43/78; A01N 43/08; A01N 43/54; A01N 43/84; A01P 13/00; C07C 237/40; C07D 233/96; C07D 265/30; C07D 277/46; C07D 307/68

(57) ABSTRACT

Provided herein are compounds that inhibit the protein-protein interaction of serine acetyltransferase (SAT) with O-acetyl serine sulfhydrylase (OASS), and can therefore be used as herbicides.

### RELATED APPLICATIONS

This application claims the benefit of priority of U.S. Provisional Patent Application No. 63/254,193 filed on 11 Oct. 2021, the contents of which are incorporated herein by reference in their entirety.

### FIELD AND BACKGROUND OF THE INVENTION

The present invention, in some embodiments thereof, relates to cysteine synthesis pathway inhibition, and more particularly, but not exclusively, to inhibitors of O-acetyl serine sulfhydrylase (OASS) enzyme and use thereof as herbicides.

Over the past decades the agrochemical industry has provided only a few new targets for pesticides, and even fewer herbicides. This depleted arsenal is problematic since due to the heavy use of existing herbicides, resistance has evolved at an accelerated pace in the last few years. This brings an urgent need to develop new herbicides, acting in a new mode of action by inhibiting novel protein targets; those to which resistance has not yet evolved. The problem is exacerbated by regulatory and voluntary deregistration of older herbicides such that fewer herbicides are available to deal with resistance. Agrochemical companies have each screened tens of thousands of new chemical entities per year in an attempt to find new herbicides. Yet the proportion of newly commercialized compounds that adequately kill plants in field situations (hits) continually decreased over the years and has reached an asymptote of near zero before 1995. Due to this situation, one can postulate that there are few or no new target sites that small organic chemicals can inhibit in the context of plant growth. Two years after glyphosate-resistant crops were introduced and widely adopted, the agrochemical industry greatly reduced its herbicide development efforts, resulting in a 90% reduction in patents issued. Most of those issued were for compounds inhibiting the same known targets by already commercialized herbicides.

Cysteine has a significant role in plant growth and development since it is the major source for the sulfur moiety in the biosynthesis of sulfur containing compounds. The cysteine biosynthetic pathway involves two sequential reactions catalyzed by serine acetyltransferase (SAT) which synthesizes the intermediary product, 0-acetyl-Ser (OAS), from acetyl-CoA and Ser, and O-acetylserine sulfhydrylase (OASS) which incorporates the sulfide to OAS producing L-cysteine. The formation of a multi-enzyme complex of OASS-SAT through the binding of the C-terminus tail of SAT to OASS is essential for cysteine biosynthesis. Thus, interference of the OASS-SAT interaction will suppress the cysteine biosynthesis pathway without affecting humans or other mammals that do not have this enzyme.

### SUMMARY

Provided herein are compounds that were designed to bind to O-acetyl serine sulfhydrylase at the binding site of serine acetyltransferase (SAT), thereby interfering with the protein-protein interaction, and affecting the cysteine synthesis pathway. According to some embodiments of the present invention, the compounds provided herein are capable of inhibiting OASS, and in general inhibit the cysteine synthesis pathway, and can therefore be used to inhibit the growth of plants. These compounds can be used as herbicides. Some compounds of the present invention can be used for seed treatment. In a particular embodiment, the compounds of the present invention are used as selective herbicides, non-selective herbicides, agricultural herbicides, non-agricultural herbicides or weed killers, herbicides in integrated pest management, herbicides in gardening, herbicides in clearing waste ground, herbicides in clearing industrial or constructions sites, or herbicides in clearing roadsides, railways and railway embankments.

In one aspect, the present invention relates to O-acetyl serine sulfhydrylase inhibitors described by the following Formula (I):

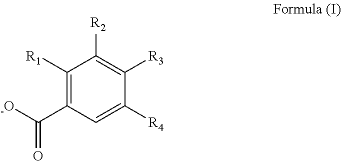

and any agronomically acceptable salt, pro-herbicide, solvate, and/or hydrate thereof,
wherein R₁ is selected from hydrogen atom, a radical of Formula (A), a radical of Formula (A2), a radical of Formula (B), and a radical of Formula (C):

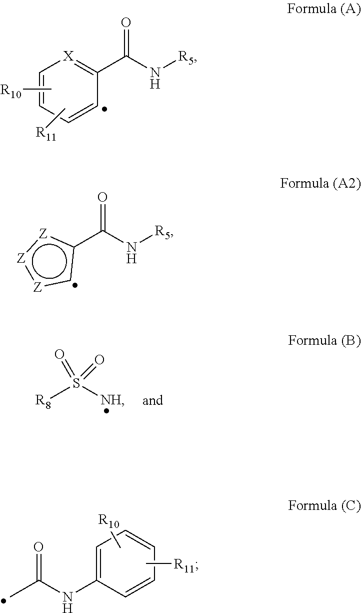

X is carbon, oxygen, nitrogen or absent (5-membered ring),
R₂ is selected from hydrogen atom, the radical of formula (D), and the following radical of Formula (E):

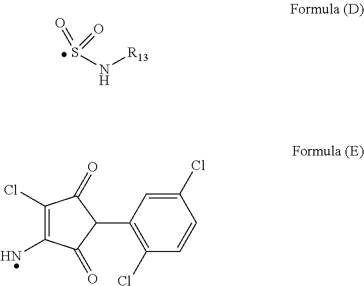

R₃ and R₄ are independently selected from hydrogen, (C₁-C₃)-alkyl, (C₁-C₃)-haloalkyl, amino, cyano, hydroxy, (C₁-C₃)-alkoxy, halogen, and nitro group; or
R₃ and R₄ are linked via (C₁-C₃)-alkyl to form a ring (for example, a cyclopenten ring fused to the benzyl ring);
R₅ is selected from any one of the following radicals:

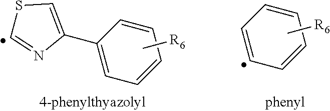

R₆ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, nitro group, and any one of the following radicals:

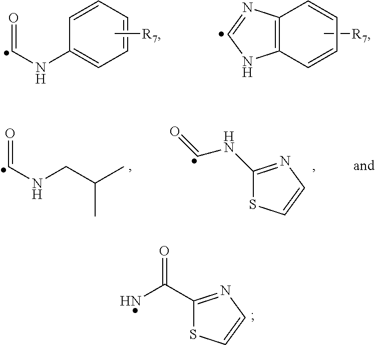

R₇ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, and nitro group;
R₈ is selected from any one of the following radicals:

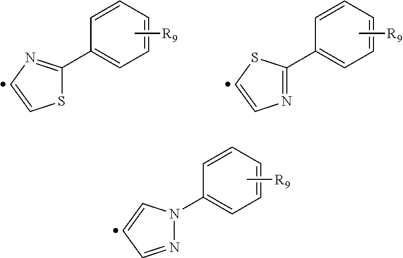

R₉ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, and nitro group;
R₁₀ and R₁₁ are attached to any available phenyl ring carbon atom and independently selected from hydrogen, (C₁-C₃)-alkyl, (C₁-C₃)-haloalkyl, (C₁-C₃)-alkoxy, halogen, amino, cyano, hydroxy, nitro group, and any one of the following radicals:

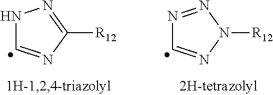

R₁₂ is selected from hydrogen, (C₁-C₃)-alkyl, and (C₁-C₃)-haloalkyl;
R₁₃ selected from any one of the following radicals:

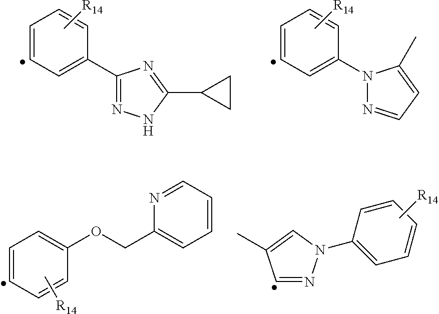

R₁₄ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, nitro group;
(i) provided that when R₁ is the radical of Formula (A), the radical of Formula (A2) or the radical of Formula (C), then
R₂, R₃ and R₄ are hydrogens;
(ii) provided that when R₁ is the radical of Formula (B), then
R₂ is hydrogen, and
R₃ and R₄ are independently selected from hydrogen, (C₁-C₃)-alkyl, (C₁-C₃)-haloalkyl, amino, cyano, hydroxy, (C₁-C₃)-alkoxy, trifluoromethyl, halogen, and nitro group;
(iii) provided that when R₂ is the radical of formula (D), then
R₁ is hydrogen, and
R₃ and R₄ are linked via (C₁-C₃)-alkyl to form a ring; and
(iv) provided that when R₂ is the radical of formula (E), then
R₁, R₃ and R₄ are hydrogens.

In some embodiments, the compound is represented by Formula (IA):

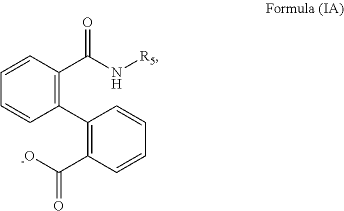

wherein R₅ is the 4-phenylthyazolyl of the following formula:

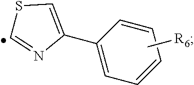

R₆ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, and nitro group.

In some embodiments, the compound is represented by the following formula:

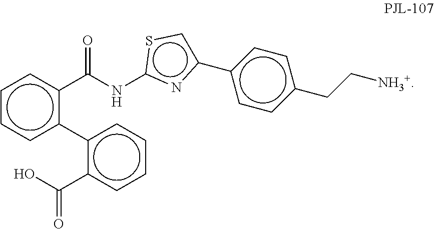

In some embodiments, the compound is represented by Formula (IA):

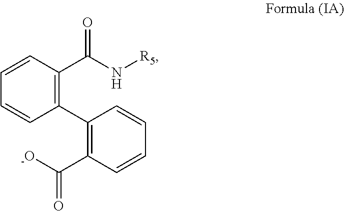

wherein R₅ is the phenyl of the following formula:

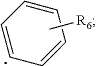

R₆ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, nitro group, and any one of the following radicals:

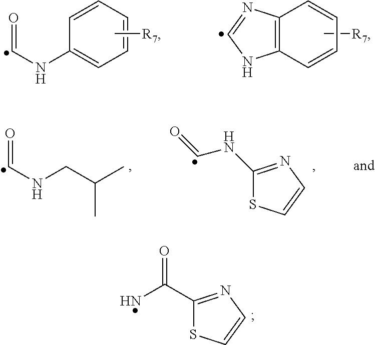

R₇ is attached to any available phenyl ring carbon atom and independently selected from hydrogen, (C₁-C₃)-alkyl, (C₁-C₃)-haloalkyl, (C₁-C₃)-alkoxy, halogen, amino, cyano, hydroxy, and nitro group.

In some embodiments, the compound is represented by Formula (IA-1):

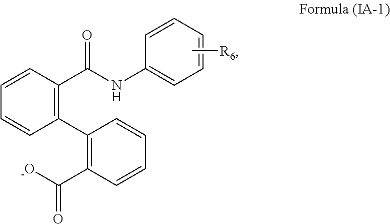

wherein R₆ is attached to any available phenyl ring carbon atom and independently selected from hydrogen, (C₁-C₃)-alkyl, (C₁-C₃)-haloalkyl, (C₁-C₃)-alkoxy, halogen, amino, cyano, hydroxy, nitro group.

In some embodiments, R₆ is selected from hydrogen, halogen, trifluoromethyl, hydroxy and methoxy.

In some embodiments, the compound is represented by Formula (IA-2):

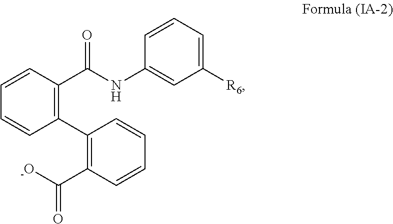

wherein R₆ is selected from the following radicals:

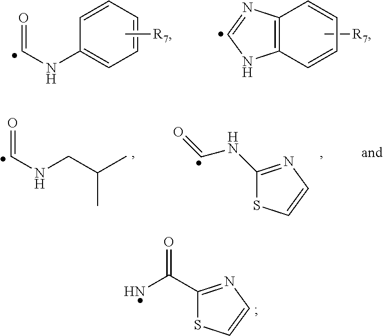

R₇ is attached to any available phenyl ring carbon atom and independently selected from hydrogen, (C₁-C₃)-alkyl, (C₁-C₃)-haloalkyl, (C₁-C₃)-alkoxy, halogen, amino, cyano, hydroxy, and nitro group.

In some embodiments, R₇ is selected from hydrogen, halogen, trifluoromethyl, hydroxy and methoxy.

In some embodiments, the compound is represented by Formula (IB):

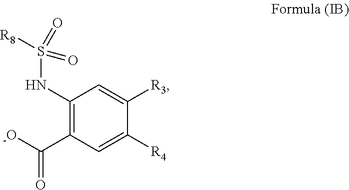

wherein R₃ and R₄ are independently selected from hydrogen, halo, hydroxy, amino, cyano, (C₁-C₃)-alkyl, (C₁-C₃)-haloalkyl, (C₁-C₃)-alkoxy, and nitro group;
R₈ is selected from any one of the following radicals:

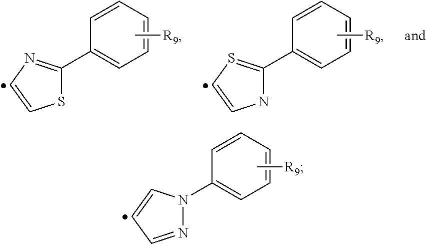

R₉ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, and nitro group.

In some embodiments, R₃, R₄ and R₉ are independently selected from hydrogen, halo, trifluoromethyl, hydroxy and methoxy.

In some embodiments, the compound is selected from:

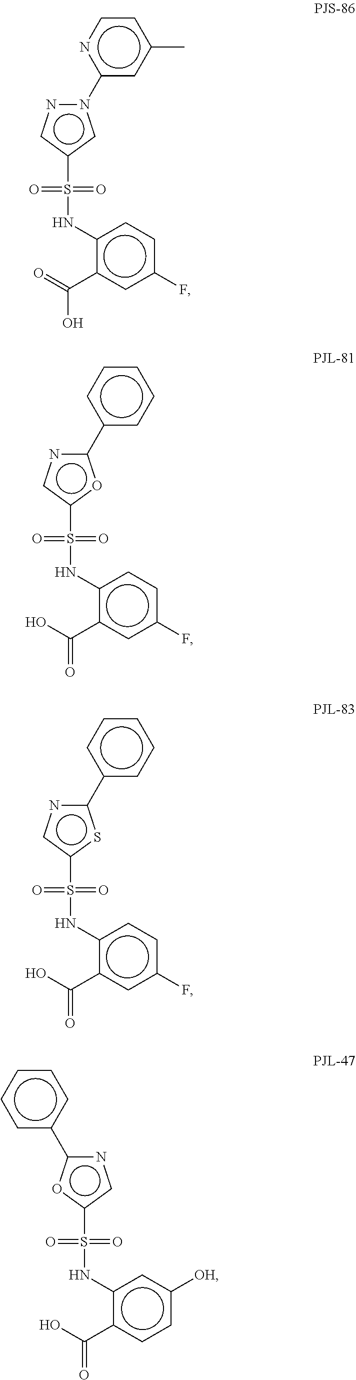

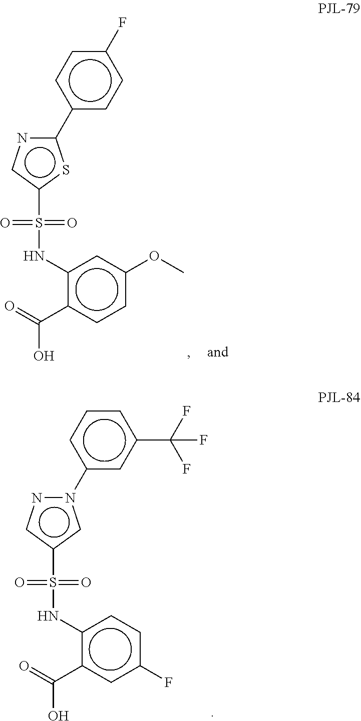

In some embodiments, the compound is represented by Formula (IC):

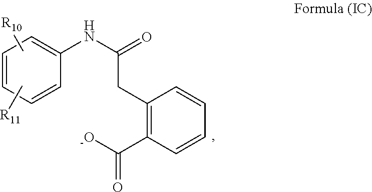

wherein R₁₀ and R₁₁ are attached to any available phenyl ring carbon atom and independently selected from hydrogen, (C₁-C₃)-alkyl, (C₁-C₃)-haloalkyl, (C₁-C₃)-alkoxy, halogen, amino, cyano, hydroxy, nitro group, and any one of the following radicals:

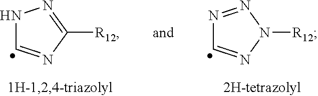

R₁₂ is selected from hydrogen, (C₁-C₃)-alkyl, and (C₁-C₃)-haloalkyl.

In some embodiments, R₁₀ and R₁₁ are independently selected from hydrogen, hydroxy, and any one of the following radicals:

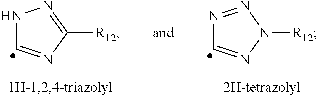

R₁₂ is selected from hydrogen, (C₁-C₃)-alkyl, and (C₁-C₃)-haloalkyl.

In some embodiments, R₁₀ is selected from hydrogen and hydroxy, and R₁₁ is selected from any one of the following radicals:

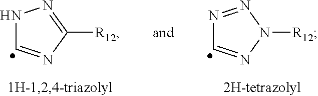

R₁₂ is selected from hydrogen, (C₁-C₃)-alkyl, and (C₁-C₃)-haloalkyl.

In some embodiments, R₁₂ is (C₁-C₃)-alkyl.

In some embodiments, the compound is represented by Formula (ID):

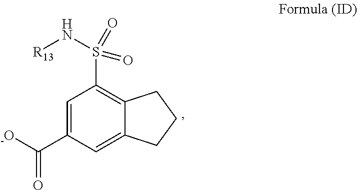

wherein R₁₃ selected from any one of the following radicals:

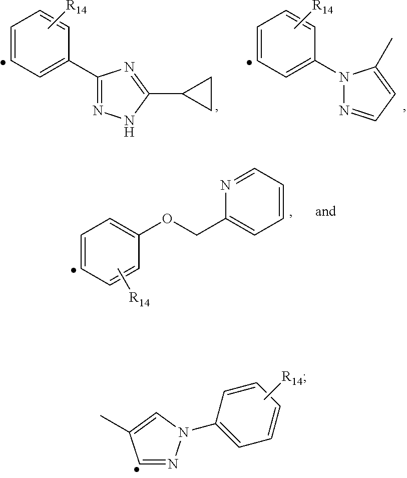

R₁₄ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, nitro group.

In some embodiments, R₁₄ is selected from hydrogen, halogen, trifluoromethyl, hydroxy and methoxy.

In some embodiments, the compound is selected from:

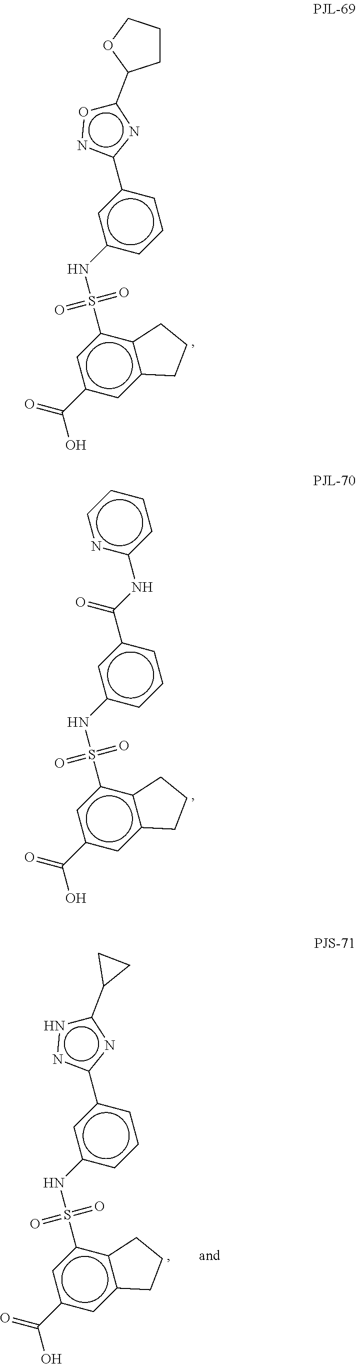

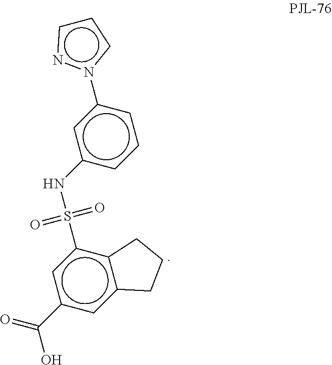

According to an aspect of some embodiments of the present invention, the compounds provided herein are listed in denotation and structure in Table 4 hereinbelow and any agronomically acceptable salt, pro-herbicide, solvate, and/or hydrate thereof.

According to an aspect of some embodiments of the present invention, there is provided a compound that inhibits the binding of O-acetyl serine sulfhydrylase (OASS) to serine acetyltransferase (SAT), which is defined by:
15-40 non-hydrogen atoms;
2-7H-bond acceptors;
0-3H-bond donors;
1-2 functional groups capable of exhibiting a negative charge;
0-1 functional groups capable of exhibiting a positive charge;
2-9 rotatable bonds;
1-2 hydrophobic functional groups;
0-4 non-aromatic rings; and
2-5 aromatic rings;
the atoms, and/or the functional groups, and/or the bonds, and/or the rings, form at least three structural determinants positioned on an arbitrary 3D cartesian coordinates system (in angstroms; Å), selected from the group consisting of:
a first hydrophobic functional group at position 78.51, 47.01, −12.92 within a radius of 1.6 (HD1);
a second hydrophobic functional group at position 78.26, 49.86, −6.08 within a radius of 1.6 (HD2);
a functional group capable of exhibiting a negative charge at position 75.93, 45.12, −12.53 within a radius of 1.7 (NC1);
an aromatic ring at position 77.12, 51.92, −10.61 within a radius of 1.6 (AR1), the aromatic ring is having a ring projection at position 80.05, 51.32, −10.93 within a radius of 2.2 (A1_{P}); and
a H-bond acceptor at position 74.18, 50.71, −13.04 within a radius of 1.6 (HA1), the H-bond acceptor is having a ring projection at position 71.80, 49.22, −11.99 within a radius of 2.2 (HA1_{P}),
and characterized by:
a Log P that ranges 0.5-5.5;
a molecular mass that ranges 300-600 g/mol;
wherein the compound exhibiting at least one property selected from the group consisting of:
k_{d} lower that 100 m as determined in vitro by an iso thermal calorimetry (ITC) assay;
IC50 lower that 200 m as determined in-vitro by a fluorescence polarization (FP) assay;
at least 10% inhibition as determined in-vitro by an FP assay; and
at least 10% inhibition as determined by an in-planta root elongation assay, and any agronomically acceptable salt, pro-herbicide, solvate, and/or hydrate thereof.

In some embodiments, the structural determinants lack atoms in at least one position selected from the group consisting of Table 2B.

In some embodiments, the structural determinants lack atoms in positions listed in Table 2B.

In some embodiments, each of the structural determinants exhibit positioning and orientation so as to interact with at least one residue in OASS, wherein:
HD1 interacts with F148, G181 and/or A228;
AR1 interacts with F148 and/or M125;
NC1, interacts with K46, T78 and/or Q147;
HA1 interacts with S75, T74 and/or Q147; and
HD2 interacts with I129, F148 and/or F230.

In some embodiments, the structural determinants exhibit positioning and orientation so as to interact with one or more residues in OASS, wherein:
HD1 interacts with F148, G181, and A228;
AR1 interacts with F148 and M125;
NC1, interacts with K46, or T78 or Q147;
HA1 interacts with S75, or T74 or Q147; and
HD2 interacts with 1129, F148 and F230.

In some embodiments, the compound provided herein is characterized by a general formula selected from the group consisting of:

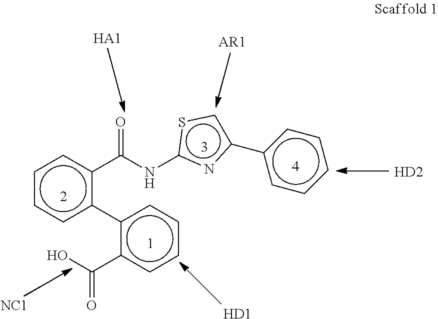

wherein rings 2 and 4 are each independently a substituted or unsubstituted 5 or 6 membered aryl or heteroaryl,

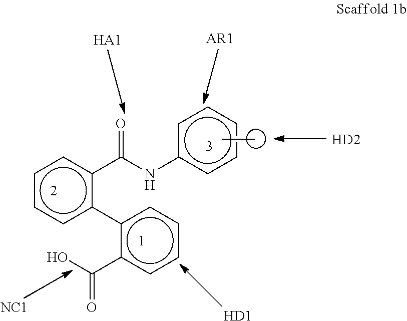

wherein ring 3 is a substituted or unsubstituted 5 or 6 membered aryl or heteroaryl,

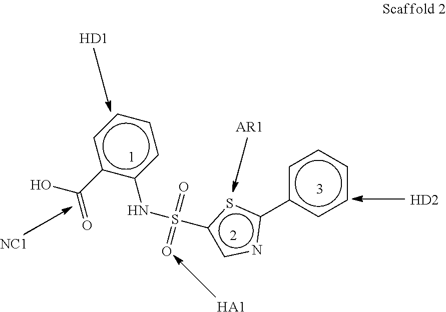

wherein ring 3 is a substituted or unsubstituted 5 or 6 membered aryl or heteroaryl,

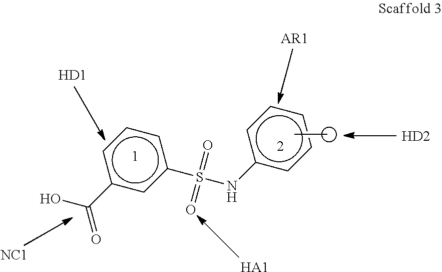

wherein ring 2 is a substituted or unsubstituted 5 or 6 membered aryl or heteroaryl,

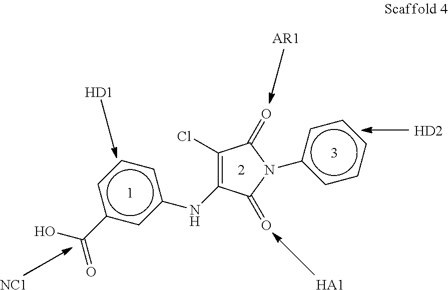

wherein ring 3 is a substituted or unsubstituted 5 or 6 membered aryl or heteroaryl, and

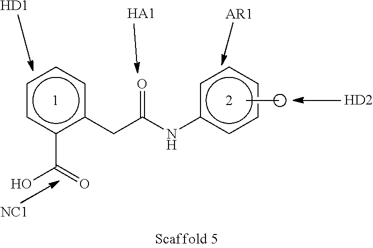

wherein ring 2 is a substituted or unsubstituted 5 or 6 membered aryl or heteroaryl.

In some embodiments, the compound under Scaffold 1 is represented by the compounds selected from the group consisting of PJL-107, PJL-106, PJL-126, PJL-110, PJL-125, PJL-130, PJL-116, PJL-65, PJL-132, PJL-61, PJL-108, PJL-60, PJL-109, PJL-118, PJL-133, PJL-119, PJL-131, PJS-212, PJL-129, PJL-115, PJL-64, PJL-127, PJL-128, PJL-67, PJL-66, PJL-59, PJL-120, PJL-117, and PJL-111;
Scaffold 1b is represented by the compounds selected from the group consisting of PJL-59, PJL-60, PJL-62, PJL-63, PJL-68, and PJS-120;
Scaffold 2 is represented by the compounds selected from the group consisting of PJL-86, PJL-81, PJS-41, PJL-114, PJL-84, PJL-47, PJL-79, PJL-83, PJL-85, PJL-5, PJL-82, PJL-27, PJS-110, PJL-121, PJL-122, and PJL-123;
Scaffold 3 is represented by the compounds selected from the group consisting of PJL-27, PJL-71, PJL-74, PJS-227, PJS-39, PJL-43, PJL-39, PJL-29, PJL-21, PJL-22, PJS-31, PJL-2, PJS-30, PJL-76, PJL-70, and PJL-69;
Scaffold 4 is represented by the compounds selected from the group consisting of PJS-211 and PJL-103; and
Scaffold 5 is represented by the compounds selected from the group consisting of PJL-95, PJL-96, PJL-91, PJL-88, PJS-208, PJL-92, and PJL-93.

In some embodiments, the compound provided herein is defined by being capable of entering a plant cell (e.g., cytosol).

In some embodiments, the compound provided herein is defined by being capable of entering a plant plastid/chloroplast.

According to an aspect of the present invention, there is provided an herbicidal composition that includes, as an active ingredient, the compound provided herein, and an agronomically acceptable carrier and optionally at least one adjuvant, the carrier or the adjuvant is for allowing the compound to be used as herbicide.

In some embodiments, the concentration of the compound is applied an herbicidal effective amount.

In some embodiments, the carrier includes at least one adjuvant, which is selected from the group consisting of a surfactant, a safener, an extender, a sticker, a synergist, an agent facilitating slow release into the environment, a drift control agent, a foaming agent, a colloid stabilizer, a dispersing agent, an emulsifying agent, a pH adjuster, a conditioning agent, a thickening agent, a wetting agent, an acidifier, a buffering agent, a water conditioner, an anti-foaming agent, a compatibility agent, a colorant and a odorant.

In some embodiments, the adjuvant facilitates an entry of the compound to a plant cell (e.g., cytosol).

In some embodiments, the adjuvant facilitates an entry of the compound to a plant plastid/chloroplast.

In some embodiments, the composition further includes an additional ingredient selected form the group consisting of an herbicide, a pesticide, a fertilizer, an insecticide, and a fungicide.

In some embodiments, the composition is in a form selected from the group consisting of a dry powder, a wettable powder, a plurality of granules, a solution, a colloid and an aerosol.

In some embodiments, the composition's type is selected from the group consisting of an emulsifiable concentrate (EC), a dispersible concentrate (DC), an emulsion in water (EW), a microemulsion (ME), an aqueous suspension concentrates (SC), an aqueous suspo-emulsion (SE), an oil dispersion (OD) and a water dispersible granule (WG).

In some embodiments, the surfactant is selected from the group consisting of a non-ionic surfactant and an anionic surfactant.

According to an aspect of the present invention, there is provided a method of inhibiting the binding of SAT to OASS, that includes contacting OASS with a compound, the compound includes functional groups at position and orientation so as to interact with at least three groups of amino acid residues of OASS, the groups of amino acid residues are selected from the group consisting of:
F148, G181 and/or A228;
F148 and/or M125;
K46, T78 and/or Q147;
S75, T74 and/or Q147; and
I129, F148 and/or F230.

In some embodiments, the groups of amino acid residues are selected from the group consisting of:
F148, G181 and A228;
F148 and M125;
K46, T78 or Q147;
S75 or T74 or Q147; and
I129, F148 and F230.

In some embodiments, the compound that is used in any of the compositions and methods provided herein, is any compound provided herein.

According to an aspect of the present invention, there is provided a method of inhibiting the post-emergence growth of a plant, that includes contacting the plant with a herbicidally effective amount of any one of the compounds or compositions provided herein.

According to an aspect of the present invention, there is provided a method of inhibiting the pre-emergence growth of a plant, that includes contacting at least one terrestrial area with a herbicidally effective amount of any one of the compounds provided herein, or with any one of the compositions provided herein.

According to an aspect of the present invention, there is provided a method of controlling undesired vegetation growth, that includes applying to at least one terrestrial area of the undesired vegetation a herbicidally effective amount of any one of the compounds provided herein, or with any one of the compositions provided herein.

In some embodiments, the terrestrial area is an agricultural area, a crop field, a garden, a greenhouse, a waste ground, an industrial or construction site, roadside railway or railway embankment.

In some embodiments, the compound or the composition provided herein, includes an herbicide for clearing waterways, canals, ponds and reservoirs, or an herbicide for clearing water weeds and algae.

In some embodiments, the compound or the compound in the composition is selected from the group consisting of a selective herbicide, a non-selective herbicide, an agricultural herbicide, a non-agricultural herbicide, an herbicide in integrated pest management, a gardening herbicide, an herbicide in clearing waste ground, an herbicide in clearing an industrial or a construction site, and an herbicide for clearing railways and railway embankments.

Various embodiments may allow various benefits, and may be used in conjunction with various applications. The details of one or more embodiments are set forth in the accompanying figures and the description below. Other features, objects and advantages of the described techniques will be apparent from the description and drawings and from the claims.

### BRIEF DESCRIPTION OF SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents an illustration of OASS's binding site for SAT C-terminal peptide, showing the molecular surface of the binding site as a white surface and showing some of the pharmacophore's structural determinants, referred to as HD1, AR1, A1_{P}, NC1, HA1, HA1_{P}, HD2, as net spheres; and

FIG. 2 presents an illustration of the OASS's binding site for SAT C-terminal peptide, showing some of the pharmacophore's structural determinants including some of the excluded positions.

### DESCRIPTION OF SOME SPECIFIC EMBODIMENTS OF THE INVENTION

The present invention, in some embodiments thereof, relates to cysteine biosynthesis pathway inhibition, and more particularly, but not exclusively, to inhibitors of O-acetyl serine sulfhydrylase (OASS) enzyme and use thereof as herbicides.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As discussed hereinabove, the agrochemical industry is in great need for new targets in plants that can be inhibited by small molecules with commercial potential to become commercial herbicides. Molecular knock-down studies with some enzymes of primary plant metabolic pathways have shown that there are some new promising herbicide targets, however, no small organic compounds were found hitherto. The present inventors have posited that some of these potential targets may not have the traditional substrate binding pocket that most herbicides possess, however there are proteins that participate in vital protein-protein interactions, and have contemplated turning these protein-protein interaction sites into targets for small molecules, which in turn can be used as herbicidal agents.

### Basis for Target Selection:

While reducing the present invention to practice, the present inventors have used computational tools that are effective for the screening small molecule as modulators (e.g., inhibitors) of protein-protein interactions (PPIs). The interface of PPIs are attractive targets for the development of selective modulators. The number of PPIs is much higher than that of single protein genes and thus entails a huge number of yet unexplored protein interfaces that play important cellular roles. In the context of pesticide development, an important advantage that is associated with PPIs is related to the evolution of resistance. Simultaneous mutations should occur on the target protein and its interactor to evade the small molecule from binding while still maintaining the vital PPI. Since the formation of two non-dependent simultaneous mutations is highly unlikely, the evolution of target site resistance will be at a vastly lower rate. Moreover, plant-specific PPIs could potentially reduce the likelihood of mammalian toxicity.

The discovery of PPI modulators in the form of small organic molecules is a challenging task, since PPI interfaces are typically relatively large (more than 800 Ain diameter) and shallow, which may not be amenable for the binding of small molecules. Moreover, the discovery of PPI modulators is often hampered by the requirement for a priori structural and experimental data, which are not abundant, particularly in the plant kingdom. This often prompted the conclusion that PPIs are “undruggable” targets. The successful approach practiced by the present inventors has led to the discovery of small molecule inhibitors targeting PPIs was to elucidate ‘hot-spots’ on the protein interface. The latter are key residues essential for the complex formation of the two protein interactors. Computer-based models, virtual screening, and molecular dynamics simulations have been used as effective tools for the elucidation of molecules that could bind a specific region on PPI interfaces. Such tools are capable of predicting the 3D structure and conformational changes in proteins and protein-ligand complexes and evaluating multiple protein conformations.

While conceiving the present invention, the present inventors have identified and selected the protein-protein interaction of serine acetyltransferase (SAT) with O-acetyl serine sulfhydrylase (OASS) (OASS-SAT) as a promising herbicidal target. It is noted herein that any reference to amino-acid numbering and position in the polypeptide chain in the context of OASS is based on the sequence numbering of O-acetyl serine sulfhydrylase from Arabidopsis thaliana (AtOASS; accession number P47998, CYSK1_ARATH, cysteine synthase 1, EC:2.5.1.47, gene: OASA1 (OAS1, OASS, OLD3), 322 amino acids). Any reference to amino-acid numbering and position in the polypeptide chain in the context of SAT is based on the sequence numbering of serine acetyltransferase (SAT) from Arabidopsis thaliana, which catalyzes the formation of O-acetyl-L-serine from acetyl-CoA and L-serine (AtSAT; accession number Q42588, SAT1_ARATH, Serine acetyltransferase 1, chloroplastic, EC:2.3.1.30, gene: SAT1 (SAT5), 314 amino acids).

While further reducing the present invention to practice, the present inventors have effectively used computational tools to elucidate the binding site of OASS and SAT, and via the compilation of the PPI pharmacophore, have successfully determined the structural blueprints for small molecular entities (inhibitors). In the process of validating and fine-tuning the PPI pharmacophore, virtual libraries comprising millions to billions of small molecules were filtered based on the meticulously complied set of criteria, and molecular dynamics and conformational changes were simulated to refine molecular features that were fitted into various docking protocols to discover novel high-affinity inhibitors of the OASS-SAT PPI.

Specifically, OASS-SAT PPI was selected for the study that led to the present invention, since the assimilation of inorganic sulfur from the environment into the various biosynthetic routes within plants is a key step for a range of cellular processes. As one of the two sulfur-containing amino acids and the first that is biosynthesized, cysteine is a precursor for the biosynthesis of a range of essential metabolites. For example, cysteine is one of the required substrates for the biosynthesis of methionine. Besides being an essential amino acid in proteins methionine plays a critical role in initiating mRNA translation and indirectly regulates various cellular processes through the precursor of S-adenosyl-methionine (SAM), the primary biological methyl group donor. Thus, the biosynthesis of cysteine has been identified by the present inventors as an essential step for plant survival.

The present inventors have identified the OASS-SAT PPI as follows: SAT C-terminal is anchored within OASS binding site. SAT 1907 carboxyl interacts with OASS Q147, N77 and T74 and directs the side chain toward the PLP cofactor in the hydrophobic pocket. SAT Ile is conserved and it was shown that a mutated peptide with Ala instead of Ile has no interaction with OASS. SAT C-terminal includes Y905 residue which directs its aromatic ring close to OASS F148 and F230 to make π-π interaction. SAT S903 forms two hydrogen bonds with M125 and K126 NH backbone at the entrance of the hydrophobic pocket.

Since cysteine is critical for a wide array of cellular processes, its biosynthesis is tightly regulated. Among the various regulators are the enzymes serine acetyltransferase (SAT, EC 2.3.1.30) and O-acetyl serine sulfhydrylase (OASS, EC 2.5.1.47). SAT catalyzes the formation of O-acetyl serine via CoA-dependent acetylation of serine. Thereafter, OASS catalyzes the L-cysteine by replacing the acetate with sulfide. The formation of a multi-enzyme complex of SAT-OASS through the binding of the C-terminus tail of SAT to OASS is essential for cysteine biosynthesis. Thus, the development of small molecule inhibitors that could inhibit this PPI has a promising herbicidal potential.

### Main Objective and Overall Methodology:

The main objective of the present invention is the provision of compounds that can inhibit the growth of plants. In the context of embodiments of the present invention, the terms “inhibiting” and “controlling undesired vegetation growth” refer to the modulation of plant growth such that the application of the compounds provided herein, i.e. in the form of a herbicidal composition comprising the compound presented herein, applied to plants through the soil (applying to a terrestrial area where growth of undesired vegetation is suspected, expected or emerged), or sprayed on the plant itself (foliage), that leads to the destruction of unwanted undesired vegetation, or damages the undesired vegetation to the point where it is no longer viable or competitive with a crop. Alternatively, the term “inhibiting”, as used herein, refers to the capacity of an herbicidal agent (the “compound”, as referred to herein), to kill or inhibit the growth of plants, preferably undesired vegetation.

In the context of some embodiments of the present invention, the term “undesired vegetation” refers also to weeds.

In the context of some embodiments of the present invention, the term “inhibiting” refers to the capacity of the compound provided herein to modulate and interfere with the binding of SAT to OASS at the PPI binding site. Consequently, the term “inhibiting” is correlated to the term “affinity” when characterizing the interaction of the compound with the target protein in the sense that the compound exhibits affinity to the SAT binding site in OASS at such a level that the compounds interfere with SAT binding, and even such that the compound can displace SAT from its binding site in OASS. In the context of embodiments of the present invention, the target is OASS, and the term “inhibiting” infers that the compound exhibits an affinity to the target that is sufficiently high to interfere with the binding of SAT to OASS, leading to inhibition of the cysteine synthesis pathway, and therefore to the inhibition of plant growth.

The compounds provided herein, and the general rules for their structural definition, have been discovered by in silico screening methods, and the validation of the rules and the activity of exemplary compounds has been carried out in a wet lab. A set of chemical requirements and preferred characteristics has been defined based on a list of all available herbicides by studying the common features of in-planta active compounds (set of chemical properties). A thorough structural analysis of the O-acetyl serine sulfhydrylase enzyme structure, and identification of potential binding sites for small molecule inhibitors was performed based on published X-ray structures PDB ID: 2ISQ (PDB DOI: 10.2210/pdb2ISQ/pdb; Francois, J. A. et al., Plant Cell, 2006, 18, 3647-3655, DOI: 10.1105/tpc.106.047316). Virtual screening was carried out based on herbicide-like profiled library of small molecules. The profiled library was generated based on an in-house database of about 30 million small organic compounds taken from various commercial sources. The database was filtered based on the above-mentioned set of chemical properties, to yield the initial set of compounds which may have the potential to be active in plants, and this reduced library of molecules was used for the virtual and in-vitro screening that yielded the pharmacophore.

### Library Construction and Molecular Filtering:

The initial chemical space constituted a database with about 30 million commercially available small molecules from various chemical vendors worldwide. Small molecules within the database were filtered based on (i) molecules that have biophysical/biochemical properties that are similar to that of known herbicides, (ii) molecules that have chemical properties resembling that of SAT C-terminal peptide known to bind OASS, and (iii) pharmacophores that were generated using OASS known binding peptides and small molecules, based on the following published articles: Francois, J. A. et al., Structural basis for interaction of O-acetyl serine sulfhydrylase and serine acetyltransferase in the Arabidopsis cysteine synthase complex. Plant Cell 18, 3647-3655 (2006); Poyraz, O. et al. Structure-guided design of novel thiazolidine inhibitors of O-acetyl serine sulfhydrylase from Mycobacterium tuberculosis. J. Med. Chem. 56, 6457-6466 (2013); Schnell, R. et al., Structural insights into catalysis and inhibition of O-acetyl serine sulfhydrylase from Mycobacterium tuberculosis. Crystal structures of the enzyme alpha-aminoacrylate intermediate and an enzyme-inhibitor complex. J. Biol. Chem. 282, 23473-23481 (2007); Huang, B. et al., The active site of O-acetyl serine sulfhydrylase is the anchor point for bienzyme complex formation with serine acetyltransferase. J. Bacteriol. 187, 3201-3205 (2005); and Spyrakis, F. et al. Fine tuning of the active site modulates specificity in the interaction of O-acetyl serine sulfhydrylase isozymes with serine acetyltransferase. Biochim. Biophys. Acta 1834, 169-181 (2013).

To filter the molecular library based on physio-chemical properties of known herbicides, the present inventors enforced the “Tice rules” as initial filters on the molecular library in order to reduce it to manageable size. Briefly, the Tice rules, described by Tice, C. M. [“Selecting the right compounds for screening: does Lipinski's Rule of 5 for pharmaceuticals apply to agrochemicals?”, Pest Manag. Sci., 2001, 57:3-16, doi: 10.1002/1526-4998(200101)57:1<3::AID-PS269>3.0.CO;2-6] is a set of simple molecular criteria that have been derived from observation and machine analysis of hundreds of herbicides, that can guide screening-compound purchase and library synthesis towards compounds that are more likely to have herbicidal activity (see, Table 4 in Tice's article).

Table 1 presents the physio-chemical properties used to select the compounds for pharmacophore determination.

TABLE 1

| Physio-chemical property | Min | Max |
|---|---|---|
| Number of non-hydrogen atoms | 15 | 40 |
| Number of hydrophobic functional groups | 1 | 2 |
| Number of aromatic rings | 1 | 3 |
| Number of non-aromatic rings | 0 | 4 |
| Number of H acceptors | 2 | 7 |
| Number of H donors | 0 | 3 |
| Number of negative functional groups | 1 | 2 |
| Number of positive functional groups | 0 | 1 |
| Number of rotatable bonds | 2 | 8 |
| LogP (calculated or empiric) | 0.5 | 5.5 |
| Molecular Weight (g/mol) | 300 | 600 |

According to some embodiments, the Min and Max values of the number of non-hydrogen atoms, and molecular weight may be larger or smaller by 10% within plausibility limits, than the values presented in Table 1. It is noted herein that the definition of the compounds provided herein, based on the pharmacophore elucidated as described herein, may encompass some known herbicides; Hence, all the known herbicides, including the herbicides listed in, for example, the databased of Gandy et al. [see, supplementary information: Gandy, M. N. et al., “An interactive database to explore herbicide physicochemical properties”, Org. Biomol. Chem. 13, 5586-5590 (2015)], are excluded from the scope of embodiments to the present invention that provide new chemical entities and from embodiments that provide new herbicides.

The OASS binding site was defined based on the crystal structure of Arabidopsis thaliana OASS in complex with SAT C-terminal peptide, published under PDB ID: 2ISQ (PDB DOI: 10.2210/pdb2ISQ/pdb; Francois, J. A. et al., Plant Cell, 2006, 18, 3647-3655, DOI: 10.1105/tpc.106.047316). In addition, bacterial OASS structures bound to various peptides or small molecules were used (e.g., PDB ID: 3ZEI; see Poyraz et al. op. cit.) to elucidate the binding site. All proteins, peptides and small molecules from these publicly accessible structures were used to generate a pharmacophore using computational tools known in the art. Small molecules fitting the pharmacophore constraints were filtered and selected for further evaluation.

### Pharmacophore Determination:

The information gathered in the computational methods and tools discussed above was used to create a set of structural determinants referred to herein as the pharmacophore of the OASS-SAT PPI binding site. Combining the list of physio-chemical properties stemming from known herbicides, with the pharmacophore of the OASS-SAT binding site allowed the present inventors to generate a plurality of novel chemical entities that can be potential herbicides, as well as identify potential herbicides in databases of known chemical entities that may never have been tested or known for exhibiting herbicidal activity.

The inventors have studied the complex structure presented in PDB ID: 2ISQ, showing the SAT C-terminus anchored within the OASS binding site, and noted that SAT amino acid 1907 carboxylate interacts with OASS amino acids Q147, N77 and T74 with the side chain located towards the pyridoxal phosphate (PLP) cofactor in a hydrophobic pocket. These interactions were taken as critical as SAT ILE is highly conserved and a mutated peptide with ALA instead of ILE abolishes OASS-SAT PPI. The SAT C-terminus includes a Y905 residue with its aromatic ring located close to OASS F148 and F230 in a π-π interaction. SAT S903 has two hydrogen bonds with OASS M125 and K126 amide backbone at the entrance of the pocket.

The pharmacophore of the OASS-SAT binding site was further determined based on peptides and small molecules that have a solved crystal structure within OASS-SAT binding site, using Biovia Discovery Studio pharmacophore generation tools as described at Barnum, D.; Greene, J.; Smellie, A.; Sprague, P. Identification of common functional configurations among molecules. J. Chem. Inf Comput. Sci. 1996, 36, 563-571. Required features were derived and selected from the analysis of all peptides and molecules and using the apparent features of the OASS in this site. Exclusion points were added to avoid overlap with OASS binding site atoms.

The process described above produced the following list of structural determinants, which constitute the pharmacophore, and presented in Table 2A-B below. The XYZ coordinates in Table 2A dictates the location a particular functional group or moiety of the compound, followed by the radius that defines the sphere within which the functional group can be placed (a quantitated degree of freedom), wherein the location of the center of the sphere is presented in angstrom (A) on an arbitrary cartesian coordinate system. For example, HD1 denotes the position of a hydrophobic group, such as an alkyl, the center of which is located at X=78.5 Å, Y=47.0 Å and Z=−12.9 Å, within a radius of 1.6 Å. Similarly, AR1 denotes the position of an aromatic ring, such as a phenyl, the center of which is located at X=77.1 Å, Y=51.9 Å and Z=−10.6 Å, within a radius of 1.6 Å, and the projection of the ring, denoted A1_{P}, is located at X=80.0 Å, Y=51.3 Å and Z=−10.9 Å, within a radius of 2.2 Å. The list of excluded positions (Table 2B) dictates where no atom of the compound can exist in order to produce a compound that binds to the binding site.

It is noted that the term “aromatic ring” encompasses aryl, heteroaryl, and multi-ring aromatic and heteroaromatic systems. It is noted that the term “hydrophobic” encompasses branched aliphatic, linear aliphatic, hetero-aliphatic, aromatic, heteroaryl, heteroaryl, and multi-ring aromatic and heteroaromatic systems. Hence, for example, HD1 can be an aromatic ring.

TABLE 2A

| Name | Denotation | X | Y | Z | Radius |
|---|---|---|---|---|---|
| Hydrophobic_1 | HD1 | 78.51 | 47.01 | −12.92 | 1.6 |
| Aromatic ring | AR1 | 77.12 | 51.92 | −10.61 | 1.6 |
| Aromatic ring | A1_{P} | 80.05 | 51.32 | −10.93 | 2.2 |
| projection | | | | | |
| Negative atom | NC1 | 75.93 | 45.12 | −12.53 | 1.7 |
| H-bond acceptor | HA1 | 74.18 | 50.71 | −13.04 | 1.6 |
| H-bond acceptor | HA1_{P} | 71.8 | 49.22 | −11.99 | 2.2 |
| projection | | | | | |
| Hydrophobic_2 | HD2 | 78.26 | 49.86 | −6.08 | 1.6 |

TABLE 2B

| Name | X | Y | Z | Radius |
|---|---|---|---|---|
| Excluded_001 | 79.08 | 41.72 | −13.52 | 1.2 |
| Excluded_002 | 79.41 | 42.72 | −14.57 | 1.2 |
| Excluded_003 | 78.46 | 42.79 | −15.05 | 1.2 |
| Excluded_004 | 79.55 | 43.58 | −13.95 | 1.2 |
| Excluded_005 | 72.62 | 45.13 | −4.48 | 1.2 |
| Excluded_006 | 72.13 | 46.26 | −5.27 | 1.2 |
| Excluded_007 | 72.73 | 46.19 | −6.67 | 1.2 |
| Excluded_008 | 73.95 | 46.28 | −6.84 | 1.2 |
| Excluded_009 | 72.63 | 47.47 | −4.49 | 1.2 |
| Excluded_010 | 73.95 | 46.98 | −3.97 | 1.2 |
| Excluded_011 | 73.66 | 45.57 | −3.52 | 1.2 |
| Excluded_012 | 71.88 | 46.01 | −7.67 | 1.2 |
| Excluded_013 | 72.38 | 45.9 | −9.03 | 1.2 |
| Excluded_014 | 71.29 | 45.87 | −10.08 | 1.2 |
| Excluded_015 | 70.4 | 45.01 | −10.05 | 1.2 |
| Excluded_016 | 73.22 | 44.64 | −9.21 | 1.2 |
| Excluded_017 | 73.57 | 44.5 | −10.59 | 1.2 |
| Excluded_018 | 72.44 | 43.42 | −8.76 | 1.2 |
| Excluded_019 | 70.83 | 45.94 | −7.48 | 1.2 |
| Excluded_020 | 72.7 | 44.42 | −11.17 | 1.2 |
| Excluded_021 | 71.36 | 46.81 | −11.01 | 1.2 |
| Excluded_022 | 70.4 | 46.88 | −12.08 | 1.2 |
| Excluded_023 | 71.1 | 46.32 | −13.31 | 1.2 |
| Excluded_024 | 70.7 | 46.57 | −14.45 | 1.2 |
| Excluded_025 | 69.95 | 48.33 | −12.31 | 1.2 |
| Excluded_026 | 71.04 | 49.18 | −12.62 | 1.2 |
| Excluded_027 | 72.15 | 47.53 | −10.96 | 1.2 |
| Excluded_028 | 71.73 | 49.17 | −11.83 | 1.2 |
| Excluded_029 | 72.15 | 45.54 | −13.07 | 1.2 |
| Excluded_030 | 72.91 | 44.95 | −14.15 | 1.2 |
| Excluded_031 | 72.85 | 43.43 | −14.23 | 1.2 |
| Excluded_032 | 71.99 | 42.8 | −13.62 | 1.2 |
| Excluded_033 | 72.44 | 45.34 | −12.06 | 1.2 |
| Excluded_034 | 73.78 | 42.85 | −14.99 | 1.2 |
| Excluded_035 | 74.5 | 43.47 | −15.48 | 1.2 |
| Excluded_036 | 74.68 | 41.28 | −12.89 | 1.2 |
| Excluded_037 | 74.95 | 40.57 | −11.64 | 1.2 |
| Excluded_038 | 75.73 | 41.45 | −10.64 | 1.2 |
| Excluded_039 | 77.08 | 41.54 | −11.08 | 1.2 |
| Excluded_040 | 75.72 | 40.84 | −9.25 | 1.2 |
| Excluded_041 | 74.94 | 42.31 | −12.99 | 1.2 |
| Excluded_042 | 77.5 | 40.57 | −11.12 | 1.2 |
| Excluded_043 | 72.66 | 41.77 | −11.76 | 1.2 |
| Excluded_044 | 67.32 | 51.12 | −9.17 | 1.2 |
| Excluded_045 | 66.47 | 52.27 | −9.43 | 1.2 |
| Excluded_046 | 65.23 | 51.83 | −10.2 | 1.2 |
| Excluded_047 | 65.32 | 51.03 | −11.13 | 1.2 |
| Excluded_048 | 67.36 | 53.18 | −10.26 | 1.2 |
| Excluded_049 | 68.71 | 52.9 | −9.7 | 1.2 |
| Excluded_050 | 68.71 | 51.4 | −9.57 | 1.2 |
| Excluded_051 | 64.27 | 52.41 | −14.57 | 1.2 |
| Excluded_052 | 63.56 | 52.26 | −15.57 | 1.2 |
| Excluded_053 | 65.31 | 54.54 | −13.76 | 1.2 |
| Excluded_054 | 66.45 | 53.96 | −13.15 | 1.2 |
| Excluded_055 | 66.26 | 53.77 | −12.13 | 1.2 |
| Excluded_056 | 65.21 | 51.54 | −14.21 | 1.2 |
| Excluded_057 | 65.51 | 50.34 | −14.98 | 1.2 |
| Excluded_058 | 64.26 | 49.73 | −15.63 | 1.2 |
| Excluded_059 | 66.19 | 49.31 | −14.08 | 1.2 |
| Excluded_060 | 67.01 | 48.26 | −14.84 | 1.2 |
| Excluded_061 | 68.16 | 49.01 | −16.02 | 1.2 |
| Excluded_062 | 68.97 | 50.23 | −15.01 | 1.2 |
| Excluded_063 | 65.76 | 51.73 | −13.31 | 1.2 |
| Excluded_064 | 68.13 | 45.76 | −17.39 | 1.2 |
| Excluded_065 | 68.96 | 45.99 | −18.58 | 1.2 |
| Excluded_066 | 68.87 | 47.05 | −19.37 | 1.2 |
| Excluded_067 | 67.99 | 48.01 | −19.12 | 1.2 |
| Excluded_068 | 69.66 | 47.16 | −20.43 | 1.2 |
| Excluded_069 | 69.69 | 45.24 | −18.83 | 1.2 |
| Excluded_070 | 67.35 | 47.94 | −18.27 | 1.2 |
| Excluded_071 | 67.93 | 48.87 | −19.76 | 1.2 |
| Excluded_072 | 70.37 | 46.39 | −20.64 | 1.2 |
| Excluded_073 | 69.59 | 48.01 | −21.07 | 1.2 |
| Excluded_074 | 65.1 | 56.49 | −9.87 | 1.2 |
| Excluded_075 | 66.05 | 57.68 | −9.97 | 1.2 |
| Excluded_076 | 66.98 | 57.67 | −10.78 | 1.2 |
| Excluded_077 | 65.8 | 58.68 | −9.13 | 1.2 |
| Excluded_078 | 66.59 | 59.9 | −9.09 | 1.2 |
| Excluded_079 | 67.86 | 59.73 | −8.28 | 1.2 |
| Excluded_080 | 68.8 | 60.54 | −8.39 | 1.2 |
| Excluded_081 | 67.9 | 58.69 | −7.45 | 1.2 |
| Excluded_082 | 69.06 | 58.43 | −6.61 | 1.2 |
| Excluded_083 | 69.99 | 57.41 | −7.25 | 1.2 |
| Excluded_084 | 71.01 | 57.04 | −6.67 | 1.2 |
| Excluded_085 | 67.05 | 58.04 | −7.4 | 1.2 |
| Excluded_086 | 69.64 | 56.96 | −8.45 | 1.2 |
| Excluded_087 | 70.45 | 56 | −9.18 | 1.2 |
| Excluded_088 | 70.85 | 54.8 | −8.35 | 1.2 |
| Excluded_089 | 70.07 | 54.31 | −7.53 | 1.2 |
| Excluded_090 | 68.73 | 57.32 | −8.9 | 1.2 |
| Excluded_091 | 72.07 | 54.32 | −8.56 | 1.2 |
| Excluded_092 | 72.56 | 53.16 | −7.82 | 1.2 |
| Excluded_093 | 72.89 | 53.51 | −6.38 | 1.2 |
| Excluded_094 | 72.94 | 52.64 | −5.51 | 1.2 |
| Excluded_095 | 73.79 | 52.57 | −8.52 | 1.2 |
| Excluded_096 | 73.43 | 51.67 | −9.68 | 1.2 |
| Excluded_097 | 72.43 | 50.27 | −9.13 | 1.2 |
| Excluded_098 | 73.59 | 49.42 | −8.08 | 1.2 |
| Excluded_099 | 73.11 | 54.79 | −6.12 | 1.2 |
| Excluded_100 | 73.41 | 55.26 | −4.77 | 1.2 |
| Excluded_101 | 72.19 | 54.96 | −3.89 | 1.2 |
| Excluded_102 | 72.33 | 54.52 | −2.75 | 1.2 |
| Excluded_103 | 73.7 | 56.76 | −4.79 | 1.2 |
| Excluded_104 | 73.06 | 55.5 | −6.92 | 1.2 |
| Excluded_105 | 71.86 | 51.18 | −3.39 | 1.2 |
| Excluded_106 | 74.2 | 51.35 | −2.65 | 1.2 |
| Excluded_107 | 74.68 | 51.03 | −4.06 | 1.2 |
| Excluded_108 | 75.2 | 50.85 | −1.6 | 1.2 |
| Excluded_109 | 75.94 | 51.75 | −4.47 | 1.2 |
| Excluded_110 | 72.14 | 51.98 | −4.05 | 1.2 |
| Excluded_111 | 79.77 | 42.88 | −5.14 | 1.2 |
| Excluded_112 | 80.63 | 42.52 | −6.25 | 1.2 |
| Excluded_113 | 81.87 | 43.38 | −6.43 | 1.2 |
| Excluded_114 | 79.83 | 42.53 | −7.55 | 1.2 |
| Excluded_115 | 78.94 | 43.73 | −7.7 | 1.2 |
| Excluded_116 | 78.26 | 43.75 | −9.03 | 1.2 |
| Excluded_117 | 78.69 | 43.09 | −9.98 | 1.2 |
| Excluded_118 | 77.17 | 44.49 | −9.12 | 1.2 |
| Excluded_119 | 78.74 | 43.1 | −5.32 | 1.2 |
| Excluded_120 | 76.83 | 45.04 | −8.27 | 1.2 |
| Excluded_121 | 76.63 | 44.55 | −10.04 | 1.2 |
| Excluded_122 | 81.83 | 44.62 | −5.97 | 1.2 |
| Excluded_123 | 82.58 | 46.95 | −6.14 | 1.2 |
| Excluded_124 | 81.62 | 47.31 | −7.22 | 1.2 |
| Excluded_125 | 80.26 | 47.08 | −7.06 | 1.2 |
| Excluded_126 | 82.07 | 47.9 | −8.39 | 1.2 |
| Excluded_127 | 79.36 | 47.45 | −8.05 | 1.2 |
| Excluded_128 | 81.18 | 48.28 | −9.39 | 1.2 |
| Excluded_129 | 79.81 | 48.05 | −9.22 | 1.2 |
| Excluded_130 | 80.94 | 44.99 | −5.49 | 1.2 |
| Excluded_131 | 82.96 | 47.22 | −14.66 | 1.2 |
| Excluded_132 | 83.38 | 47.14 | −13.21 | 1.2 |
| Excluded_133 | 83.65 | 48.17 | −12.59 | 1.2 |
| Excluded_134 | 83.44 | 45.92 | −12.67 | 1.2 |
| Excluded_135 | 83.88 | 45.73 | −11.29 | 1.2 |
| Excluded_136 | 83.28 | 44.44 | −10.66 | 1.2 |
| Excluded_137 | 81.78 | 44.5 | −10.66 | 1.2 |
| Excluded_138 | 83.17 | 45.07 | −13.26 | 1.2 |
| Excluded_139 | 81.37 | 60.45 | −15.18 | 1.2 |
| Excluded_140 | 80.23 | 61.08 | −15.82 | 1.2 |
| Excluded_141 | 79.31 | 60.02 | −16.42 | 1.2 |
| Excluded_142 | 79.59 | 58.83 | −16.33 | 1.2 |
| Excluded_143 | 81.61 | 59.43 | −15.4 | 1.2 |
| Excluded_144 | 78.2 | 60.43 | −17.04 | 1.2 |
| Excluded_145 | 77.27 | 59.47 | −17.64 | 1.2 |
| Excluded_146 | 77.64 | 59.02 | −19.05 | 1.2 |
| Excluded_147 | 75.95 | 60.23 | −17.61 | 1.2 |
| Excluded_148 | 76.39 | 61.63 | −17.87 | 1.2 |
| Excluded_149 | 77.59 | 61.77 | −16.98 | 1.2 |
| Excluded_150 | 77.48 | 57.73 | −19.3 | 1.2 |
| Excluded_151 | 75.86 | 55.81 | −19.94 | 1.2 |
| Excluded_152 | 79.3 | 55.56 | −18.24 | 1.2 |
| Excluded_153 | 79.24 | 53.99 | −19.75 | 1.2 |
| Excluded_154 | 79.35 | 54.41 | −17.6 | 1.2 |
| Excluded_155 | 79.31 | 53.44 | −18.5 | 1.2 |
| Excluded_156 | 77.13 | 57.1 | −18.51 | 1.2 |
| Excluded_157 | 79.31 | 56.53 | −17.78 | 1.2 |
| Excluded_158 | 76.5 | 50.23 | −20.99 | 1.2 |
| Excluded_159 | 76.11 | 49.43 | −19.83 | 1.2 |
| Excluded_160 | 77.09 | 48.31 | −19.55 | 1.2 |
| Excluded_161 | 74.74 | 48.8 | −20.03 | 1.2 |
| Excluded_162 | 73.6 | 49.79 | −20.17 | 1.2 |
| Excluded_163 | 72.27 | 49.11 | −20.02 | 1.2 |
| Excluded_164 | 71.23 | 49.69 | −20.3 | 1.2 |
| Excluded_165 | 72.29 | 47.86 | −19.58 | 1.2 |
| Excluded_166 | 73.23 | 47.39 | −19.34 | 1.2 |
| Excluded_167 | 71.38 | 47.32 | −19.45 | 1.2 |
| Excluded_168 | 77.44 | 48.15 | −18.27 | 1.2 |
| Excluded_169 | 78.35 | 47.1 | −17.88 | 1.2 |
| Excluded_170 | 79.7 | 47.61 | −17.42 | 1.2 |
| Excluded_171 | 80.43 | 46.92 | −16.7 | 1.2 |
| Excluded_172 | 77.03 | 48.8 | −17.53 | 1.2 |
| Excluded_173 | 80.04 | 48.82 | −17.83 | 1.2 |
| Excluded_174 | 81.31 | 49.42 | −17.44 | 1.2 |
| Excluded_175 | 81.13 | 50.88 | −17.03 | 1.2 |
| Excluded_176 | 80 | 51.36 | −16.89 | 1.2 |
| Excluded_177 | 79.37 | 49.37 | −18.46 | 1.2 |
| Excluded_178 | 82.24 | 51.58 | −16.83 | 1.2 |
| Excluded_179 | 82.18 | 52.98 | −16.44 | 1.2 |
| Excluded_180 | 81.42 | 53.22 | −15.14 | 1.2 |
| Excluded_181 | 80.45 | 53.99 | −15.11 | 1.2 |
| Excluded_182 | 81.86 | 52.58 | −14.06 | 1.2 |
| Excluded_183 | 81.22 | 52.72 | −12.76 | 1.2 |
| Excluded_184 | 81.44 | 54.11 | −12.16 | 1.2 |
| Excluded_185 | 80.79 | 54.47 | −11.19 | 1.2 |
| Excluded_186 | 81.73 | 51.65 | −11.8 | 1.2 |
| Excluded_187 | 82.71 | 51.94 | −14.15 | 1.2 |
| Excluded_188 | 84.14 | 55.14 | −10.68 | 1.2 |
| Excluded_189 | 84.98 | 55.02 | −9.48 | 1.2 |
| Excluded_190 | 85.44 | 52.79 | −10.24 | 1.2 |
| Excluded_191 | 84.1 | 55.02 | −8.22 | 1.2 |
| Excluded_192 | 83.28 | 53.78 | −8.04 | 1.2 |
| Excluded_193 | 83.82 | 52.66 | −7.42 | 1.2 |
| Excluded_194 | 81.97 | 53.74 | −8.48 | 1.2 |
| Excluded_195 | 83.05 | 51.51 | −7.24 | 1.2 |
| Excluded_196 | 81.19 | 52.59 | −8.31 | 1.2 |
| Excluded_197 | 81.74 | 51.48 | −7.69 | 1.2 |
| Excluded_198 | 84.11 | 54.33 | −11.37 | 1.2 |

The structural determinants of the pharmacophore can be mapped to their corresponding enzyme counterparts, namely each pharmacophore position interacts with one or more atoms in the OASS enzyme, as follows, using the residue numbering adopted from the structure of AtOASS, PDB ID: 2ISQ:
HD1 interacts with F148, G181 and A228;
AR1 interacts with F148 and M125;
NC1, interacts with K46, T78 or Q147;
HA1 interacts with S75 or T74 or Q147; and
HD2 interacts with I129, F148 and F230.

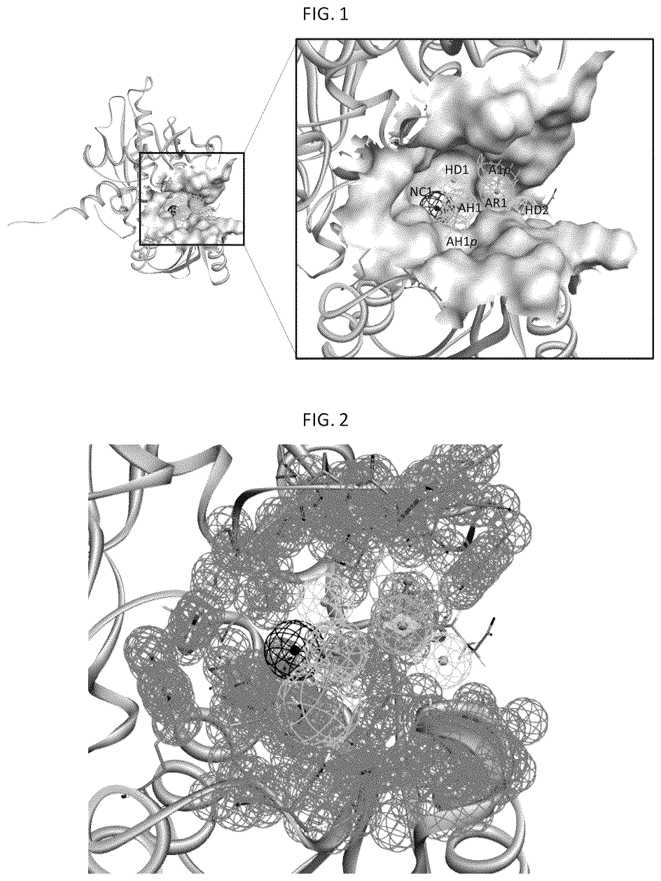

FIG. 1 presents an illustration of OASS's binding site for SAT C-terminal peptide, showing the molecular surface of the binding site as a white surface and showing some of the pharmacophore's structural determinants, referred to as HD1, AR1, A1_{P}, NC1, HA1, HA1_{P}, HD2, as net spheres.

FIG. 2 presents an illustration of the OASS's binding site for SAT C-terminal peptide, showing some of the pharmacophore's structural determinants including some of the excluded positions.

### Pharmacophore Validation:

Based on the virtual screening described above, 150 molecules having a high consensus score, were selected for in vitro evaluation for their ability to bind to OASS and to compete with SAT binding; this ability is referred to herein as “herbicidal activity” and a high rate of molecules that exhibit herbicidal activity would validate the pharmacophore hypothesis.

The in vitro evaluation of herbicidal activity included isothermal titration calorimetry (ITC) and fluorescence polarization (FP) assays.

Briefly, ITC is a quantitative bioanalytical technique that can determine enthalpy changes (ΔH) and binding stoichiometry (N) of the molecular interaction between two or more molecules in solution, such as enzyme and its inhibitor, as in the present case. ITC also allows to determine association constant (Ka), which is reciprocal to dissociation constant or binding affinity (Kd). From these initial measurements, Gibbs free energy changes (ΔG) and entropy changes (−TΔS) are calculated using the Gibbs free energy equation. ITC assays are used in the drug discovery process to determine the thermodynamic parameters of a compound-ligand interaction in solution, and the principle is based on the measurement of the heat released or absorbed as the result of a chemical reaction such as the binding event of a compound to its ligand. During the ITC screening, one component of the reaction is contained in a temperature-controlled cell, then the binding partner is gradually titrated into the system, and when binding occurs, heat is either released or absorbed. This heat change is directly proportional to the amount of binding; hence, this technique is suitable as a secondary screen yielding not only the binding affinity but also the thermodynamic profile of a ligand-target interaction giving insight into the structure-function relationship on the molecular level.

The technique of fluorescence polarization (FP) is based on the observation that when a fluorescently labeled molecule is excited by polarized light, it emits light with a degree of polarization that is inversely proportional to the rate of molecular rotation. This property of fluorescence can be used to measure the interaction of a small labeled ligand with a larger protein and provides a basis for direct and competition binding assays. FP assays are readily adaptable to a high-throughput format, have been used successfully in screens directed against a wide range of targets, and are particularly valuable in screening for inhibitors of protein-protein and protein-nucleic acid interactions when a small binding epitope can be identified for one of the partners. Competition of OASS-SAT complex by a small molecule observed via FP demonstrated that the molecules' affinity to the OASS-SAT binding site is strong enough to disrupt already conjugated OASS-SAT complex.

To evaluate the ability of the molecules to bind AtOASS (O-acetyl serine sulfhydrylase from Arabidopsis thaliana), the protein was placed in the ITC syringe and was titrated with the molecules that were placed in the ITC cell (see Examples section below). The direct dissociation binding constant (Kd) was then extracted from the binding curve using a single binding model, wherein a low Kd value corresponds to a stronger binding affinity.

Molecules were also tested for their ability to compete with the OASS binding to the SAT-derived peptide via a fluorescence polarization (FP) competition assay wherein the N-terminus labeled peptide was incubated with the AtOASS (see Examples section below). High FP values correspond to the peptide-bound state (low molecule concentrations) and low FP values correspond to the peptide-free state (low FP values). To extract the IC50, the FP data were fitted to a single site inhibition model in PRISM by GraphPad Software. The corresponding IC50 values were extracted, whereas low IC50 values correspond to molecule that better competes with the peptide binding to AtOASS.

### Discovery of Novel Herbicides:

The elucidation of the molecular blueprints of herbicides, namely the physio-chemical properties of known herbicides combined with the structural determinants constituting the pharmacophore hypothesis that inhibits the formation of the OASS-SAT complex, according to some embodiments of the present invention, can be harnesses to discover herbicides among known molecules (known molecules that their herbicidal activity was not known) as well as to design and generate novel molecules which will exhibit herbicidal activity, both manually/visually and computationally. A skilled artisan of the art of molecular design and computational chemistry possesses the ability to scan the chemical space of plausible scaffold for molecules that fit the molecular blueprints of herbicides provided herein, and select a number of suitable scaffolds to be used as a starting point. The process can be effected both manually/visually and computationally, using software packages such as BIOVIA Discovery Studio [BIOVIA, Dassault Systemes, BIOVIA Workbook, Release 2020; BIOVIA Pipeline Pilot, Release 2020, San Diego: Dassault Systemes, 2022].

Briefly, the generation of de novo compounds based on the herein-provided molecular blueprints may start, for example, from seeking a common scaffold within a list of compounds that exhibited high affinity to the target either in silico, in vitro or in planta. The common scaffold is thereafter used to place the structural determinants of the pharmacophore in the corresponding positions for each. The generated structurers are thereafter screened in silico for their docking or consensus score to filter-out misfits.

Hence, according to some embodiments of the present invention, there is provided a compound, which is an inhibitor of the O-acetyl serine sulfhydrylase (OASS) enzyme, at least in the sense of interfering with the binding of the SAT peptide to its binding site in OASS, which is defined by:
15-40, or 20-50 non-hydrogen atoms;
2-7H-bond (hydrogen bond) acceptors;
0-3H-bond donors;
1-2 functional groups capable of exhibiting a negative charge (e.g., carboxyl group);2-8 rotatable bonds;
0-1 functional groups capable of exhibiting a positive charge (e.g., carboxyl group);2-8 rotatable bonds;
2-9 rotatable bonds;
0-2 hydrophobic functional groups (e.g., alkyl or aryl group); and
0-4 non-aromatic rings;
2-5 aromatic rings;
   whereas at least some of the atoms constitute at least three, or at least four, or all five structural features (determinants) positioned and oriented on an arbitrary 3D cartesian coordinates system (in angstroms; Å), selected from the group consisting of:
i) a functional group capable of exhibiting a negative charge at position 75.93, 45.12, −12.53 within a radius of 1.7;
ii) a first hydrophobic functional group at position 78.51, 47.01, −12.92 within a radius of 1.6;
iii) a second hydrophobic functional group at position 78.26, 49.86, −6.08 within a radius of 1.6;
iv) an aromatic ring at position 77.12, 51.92, −10.61 within a radius of 1.6, whereas this aromatic ring is having a ring projection at position 80.05, 51.32, −10.93 within a radius of 2.2; and
v) a H-bond acceptor at position 74.18, 50.71, −13.04 within a radius of 1.6, whereas this H-bond acceptor is having a ring projection at position 71.80, 49.22, −11.99 within a radius of 2.2.

The compound is also characterized by:
a Log P (empiric or calculated) that ranges 0.5-5.5; and
a molecular mass that ranges 300-600 g/mol.

The compound is also characterized by exhibiting at least one property selected from the group consisting of:
k_{d} lower that 100 μm, or lower that 80 μm, or lower that 50 μm, or lower that 30 μm, or lower that 10 m as determined in vitro by an iso thermal calorimetry (ITC) assay;
IC50 lower that 200 μm, or lower that 100 μm, or lower that 50 μm, or lower that 10 μm as determined in-vitro by a fluorescence polarization (FP) assay;
at least 10%, or at least 20%, or at least 30%, or at least 40%, or at least 50%, or at least 60%, or at least 70%, or at least 80%, or at least 90% inhibition as determined in-vitro by an FP assay; and at least 10%, or at least 20%, or at least 30%, or at least 40%, or at least 50%, or at least 60%, or at least 70%, or at least 80%, or at least 90% inhibition as determined by an in-planta root elongation assay.

In addition to the above, while considering the compound in its OASS-bound configuration, the compound is defined also by lacking atoms in the positions presented in Table 2B hereinabove, listing excluded positions. This is to say that the compound has no atoms that would prevent the binding to OASS due to steric hindrance, space occupation, or overlap with OASS atoms.

Each of the structural determinants that defines the compound, according to some embodiments of the present invention, exhibit positioning and orientation so as to interacts with one or more residues in the OASS. Specifically, as determined by the pharmacophore: The structural determinant referred to herein as HD1 interacts with F148, G181 and/or A228; The structural determinant referred to herein as AR1 interacts with F148 and/or M125; The structural determinant referred to herein as NC1, interacts with K46, T78 and/or Q147; The structural determinant referred to herein as HA1 interacts with S75 or, T74 and/or Q147; and

The structural determinant referred to herein as HD2 interacts with 1129, F148 and/or F230.

In some embodiments, HD1 interacts with F148, G181, and A228; AR1 interacts with F148 and M125; NC1, interacts with K46, or T78 or Q147; HA1 interacts with S75, or T74 or Q147; and HD2 interacts with 1129, F148 and F230.

To find new molecules that fit to the elucidate pharmacophore, for each active compound/hit (validated in-vitro), the inventors identified a core scaffold, and based on this scaffold identified additional available library of building blocks to enumerate and generate novel compounds, with similar scaffold but which differ from the hit by replacing chemical groups (modifying/adding/removing) in order to get the best fit to the described pharmacophore. This process was done computationally following the method described in Pierce, A. C.; Rao G.; Bemis G. W. BREED: Generating novel inhibitors through hybridization of known ligands. Application to CDK2, P38, and HIV protease. J. Med. Chem. 2004, 47, 2768-2775. and Lewell, X. Q.; Judd, D. B.; Watson, S. P.; Hann, M. M. “RECAP—retrosynthetic combinatorial analysis procedure: a powerful new technique for identifying privileged molecular fragments with useful applications in combinatorial chemistry.” J. Chem. Inf. Comput. Sci. 1998, 38,511-522, and the top ranked compounds are selected for chemical synthesis.

In order to reduce to practice the elucidation of the molecular blueprints of herbicides, the inventors have selected a single representative molecular scaffold that was common to 5 library molecules that were used to validate the pharmacophore hypothesis.

Table 3 presents the chemical structures of the 5 representative library molecules that were used to validate the pharmacophore hypothesis alongside with the mapping of the pharmacophore on their core scaffold. The pharmacophore's structural determinants are marked as denoted in Table 2A.

TABLE 3

| Molecular in screening library | | Pharmacophore mapping on core scaffold |
|---|---|---|
| PJL-61 | 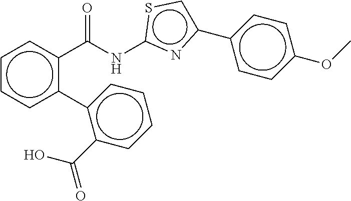 | 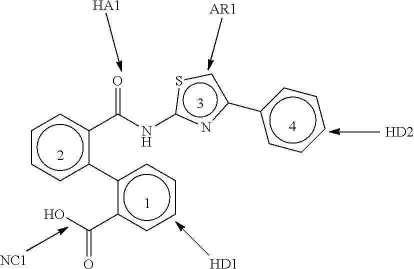 |
| | | Scaffold 1 |
| PJL-63 | 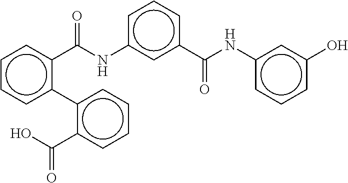 | 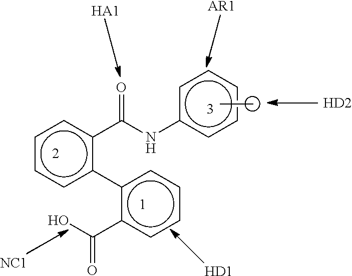 |
| | | Scaffold 1b |
| PJS-41 | 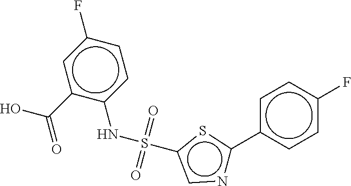 | 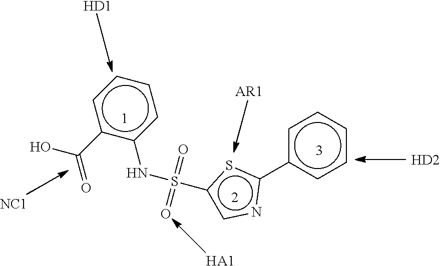 |
| | | Scaffold 2 |
| PJS-39 | 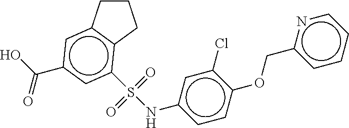 | 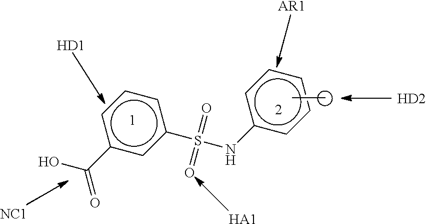 |
| | | Scaffold 3 |
| PJS-211 | 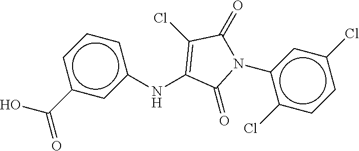 | 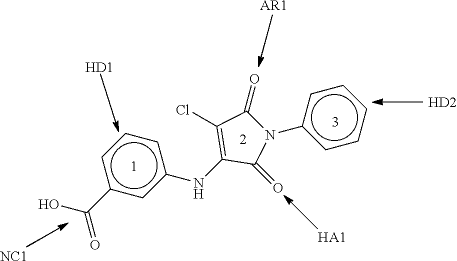 |
| | | Scaffold 4 |
| PJL-93 | 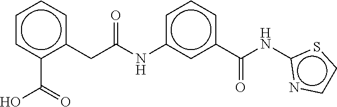 | 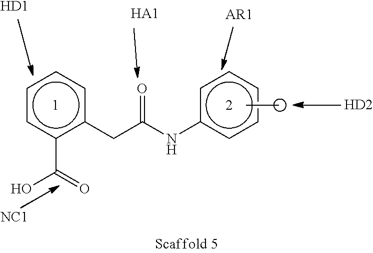 |
| | | Scaffold 5 |

Whereas rings 2 and 4 in Scaffold 1 are each independently a substituted or unsubstituted 5 or 6 membered aryl or heteroaryl, ring 3 in Scaffold 1b is a substituted or unsubstituted 5 or 6 membered aryl or heteroaryl, ring 3 in Scaffold 2 is a substituted or unsubstituted 5 or 6 membered aryl or heteroaryl, ring 2 in Scaffold 3 is a substituted or unsubstituted 5 or 6 membered aryl or heteroaryl, ring 3 in Scaffold 4 is a substituted or unsubstituted 5 or 6 membered aryl or heteroaryl, and ring 2 in Scaffold 5 is a substituted or unsubstituted 5 or 6 membered aryl or heteroaryl.

The following compound, which have been found effecting as herbicide, fall under one of the scaffolds 1-5:
PJL-107, PJL-106, PJL-126, PJL-110, PJL-125, PJL-130, PJL-116, PJL-65, PJL-132, PJL-61, PJL-108, PJL-60, PJL-109, PJL-118, PJL-133, PJL-119, PJL-131, PJS-212, PJL-129, PJL-115, PJL-64, PJL-127, PJL-128, PJL-67, PJL-66, PJL-59, PJL-120, PJL-117, and PJL-111 fall under Scaffold 1;
Scaffold 1b is represented by the compounds selected from the group consisting of PJL-59, PJL-60, PJL-62, PJL-63, PJL-68, and PJS-120;
PJL-86, PJL-81, PJS-41, PJL-114, PJL-84, PJL-47, PJL-79, PJL-83, PJL-85, PJL-5, PJL-82, PJL-27, PJS-110, PJL-121, PJL-122, and PJL-123 fall under Scaffold 2;
PJL-27, PJL-71, PJL-74, PJS-227, PJS-39, PJL-43, PJL-39, PJL-29, PJL-21, PJL-22, PJS-31, PJL-2, PJS-30, PJL-76, PJL-70, and PJL-69 fall under Scaffold 3;
PJS-211 and PJL-103 fall under Scaffold 4; and
PJL-95, PJL-96, PJL-91, PJL-88, PJS-208, PJL-92, and PJL-93 fall under Scaffold 5.

The identification of molecules from a virtual screening library, the extraction of active molecules from the hits of the virtual screening, and the elucidation of the pharmacophore provided a core scaffold in the form of a benzoic acid core (benzyl ring corresponds to HD1 structural determinant) with four ring substitutions besides the carboxyl functional group (carboxyl corresponds to NC1 structural determinant). It is noted herein that the selection of the scaffold should not be seen as limiting the scope of the present inventing to herbicides having this particular scaffold, and it should be understood that the present invention is capable of providing many more herbicides with other scaffolds, based on the same paradigm, the same pharmacophore hypothesis and the same screening procedure.

According to some embodiments of the present invention, the herbicidal compound comprises a benzoic acid core and four variables in aromatic ring positions 2, 3, 4 and 5, referred to as R₁, R₂, R₃ and R4 respectively; the scaffold is referred to herein by the general Formula I:

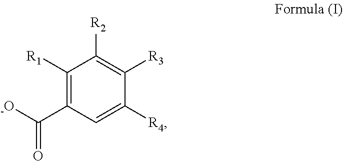

wherein R1 is selected from hydrogen atom, a radical of Formula (A), a radical of Formula (A2), a radical of Formula (B), and a radical of Formula (C):

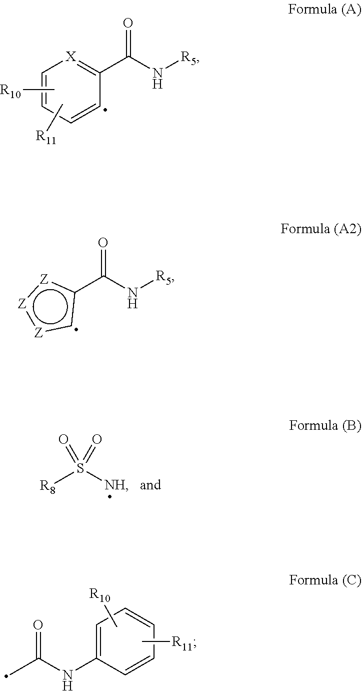

X is carbon, oxygen, nitrogen or absent (5-membered ring),
R₂ is selected from hydrogen atom, the radical of formula (D), and the following radical of Formula (E):

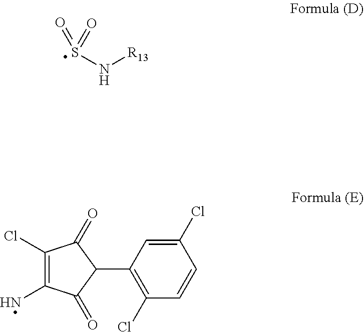

R₃ and R₄ are independently selected from hydrogen, (C₁-C₃)-alkyl, (C₁-C₃)-haloalkyl, amino, cyano, hydroxy, (C₁-C₃)-alkoxy, halogen, and nitro group; or
R₃ and R₄ are linked via (C₁-C₃)-alkyl to form a ring;
R₅ is selected from any one of the following radicals:

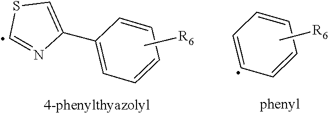

R₆ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, nitro group, and any one of the following radicals:

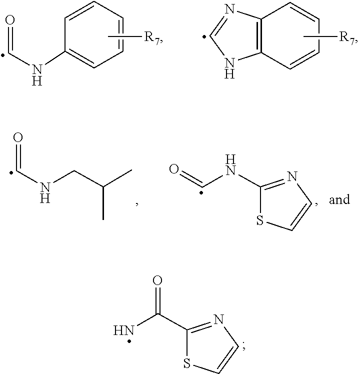

R₇ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, and nitro group;
R₈ is selected from any one of the following radicals:

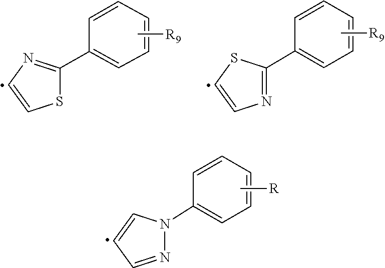

R₉ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, and nitro group;
R₁₀ and R₁₁ are attached to any available phenyl ring carbon atom and independently selected from hydrogen, (C₁-C₃)-alkyl, (C₁-C₃)-haloalkyl, (C₁-C₃)-alkoxy, halogen, amino, cyano, hydroxy, nitro group, and any one of the following radicals:

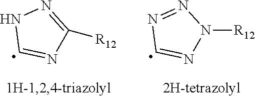

R₁₂ is selected from hydrogen, (C₁-C₃)-alkyl, and (C₁-C₃)-haloalkyl;
R₁₃ selected from any one of the following radicals:

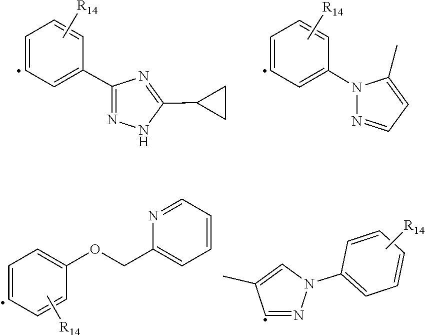

R₁₄ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, nitro group;
(i) provided that when R₁ is the radical of Formula (A), the radical of Formula (A2) or the radical of Formula (C), then
R₂, R₃ and R₄ are hydrogens;
(ii) provided that when R₁ is the radical of Formula (B), then
R₂ is hydrogen, and
R₃ and R₄ are independently selected from hydrogen, (C₁-C₃)-alkyl, (C₁-C₃)-haloalkyl, amino, cyano, hydroxy, (C₁-C₃)-alkoxy, trifluoromethyl, halogen, and nitro group;
(iii) provided that when R₂ is the radical of formula (D), then
R₁ is hydrogen, and
R₃ and R₄ are linked via (C₁-C₃)-alkyl to form a ring; and
(iv) provided that when R₂ is the radical of formula (E), then
R₁, R₃ and R₄ are hydrogens.

While the structure in general Formula (I) is shown as an anion of a carboxylic (benzoic) acid, it is to be understood that the compound can be in the free acid form (—COOH) as well as any salt or ester thereof. Hence, any depiction, illustration or scheme showing the compounds provided herein as an anion or a free acid, it is to be taken as both.

The term “alkyl” refers to a saturated monovalent hydrocarbon radical. Exemplary alkyl groups include methyl, ethyl and propyl. The term “(C₁-C₃)-alkyl” refers to an alkyl containing from one to three carbon atoms. When alkyl is used as a suffix following another named group, such as “haloalkyl”, this is intended to refer to an alkyl having bonded thereto one, two or three of the other, specifically-named groups, such as halogen, at any point of attachment on either the straight or branched chain of the alkyl.

The term “aryl”, refers to a monovalent unsaturated aromatic hydrocarbon radical of six to eighteen ring atoms having a single ring or multiple condensed rings. Exemplary aryl groups are phenyl, biphenyl, benzyl, naphthyl, anthryl, pyrenyl and the like. When the term “substituted” is used with such groups, as in “optionally substituted with one to three substituents independently selected from”, it should be understood that the aryl moiety may be optionally substituted with the same or different groups independently selected from those recited above and hereinafter as appropriate.

The term “cycloalkyl” refers to a fully saturated and partially unsaturated cyclic monovalent hydrocarbon radical having three to six carbon atoms in a ring. Exemplary cycloalkyl groups are cyclopropyl, cyclobutyl, cyclopentyl and cyclohexanyl.

The terms “heterocyclic” and “heterocyclyl” refer to fully saturated or partially unsaturated non-aromatic cyclic radicals of three to eight ring atoms in each cycle (in each monocyclic group, six to twelve atoms in a bicyclic group, and ten to eighteen atoms in a tricyclic group), which have at least one heteroatom (nitrogen, oxygen or sulfur) and at least one carbon atom in a ring. Each ring of the heterocyclic group containing a heteroatom may have from one to three heteroatoms, where the nitrogen and/or sulfur heteroatoms may optionally be oxidized and the nitrogen heteroatoms may optionally be quaternised. A heterocyclyl group may have a carbon ring atom replaced with a carbonyl group. The heterocyclyl group may be attached to the remainder of the molecule at any nitrogen atom or carbon atom of the ring or ring system. Additionally, the heterocyclo group may have a second or third ring attached thereto in a spiro or fused fashion, provided the point of attachment is to the heterocyclyl group. An attached spiro ring may be a carbocyclic or heterocyclic ring and the second and/or third fused ring may be a cycloalkyl, aryl or heteroaryl ring. Exemplary monocyclic heterocyclic groups include azetidinyl, oxiranyl, pyrrolidinyl, pyrazolinyl, imidazolidinyl, dioxanyl, dioxolanyl, oxazolidinyl, piperidinyl, piperazinyl, morpholinyl, tetrahyrdofuryl, tetrahydropyranyl, thiamorpholinyl, and the like. Exemplary bicyclic heterocyclic groups include indolinyl, isoindolinyl, quinuclidinyl, benzopyrrolidinyl, benzopyrazolinyl, benzoimidazolidinyl, benzopiperidinyl, tetrahydroisoquinolinyl, tetrahydroquinolinyl, dihydroisoindolyl and the like.

The term “heteroaryl” refers to aromatic monocyclic, bicyclic or tricyclic radicals of three to eight ring atoms in each cycle (for example, three to eight atoms in a monocyclic group, six to twelve atoms in a bicyclic group, and to eighteen atoms in a tricyclic group), which have at least one heteroatom (nitrogen, oxygen or sulfur) and at least one carbon atom in a ring. Each ring of the heteroaryl group may have one to four heteroatoms, wherein nitrogen and/or sulfur may optionally be oxidised, and the nitrogen heteroatoms may optionally be quaternised. The heteroaryl group may be attached to the remainder of the molecule at any nitrogen atom or carbon atom of the ring or ring system. Additionally, the heteroaryl group may have a second or third carbocyclic (cycloalkyl or aryl) or heterocyclic ring fused thereto provided the point of attachment is to the heteroaryl group. Exemplary heteroaryl groups are pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl and so on. Exemplary bicyclic heteroaryl groups include benzothiazolyl, benzoxazolyl, quinolinyl, benzoxadiazolyl, benzothienyl, chromenyl, indolyl, indazolyl, isoquinolinyl, benzimidazolyl, benzopyranyl, benzofuryl, benzofurazanyl, benzopyranyl, cinnolinyl, quinoxalinyl, pyrrolopyridyl, furopyridinyl (such as furo[2,3-c]pyridinyl, furo[3,2-b]pyridinyl] or furo[2,3-b]pyridinyl), triazinylazepinyl, and the like.

The heterocyclyl ring may optionally be fused to a (one) aryl or heteroaryl ring as defined herein provided the aryl and heteroaryl rings are monocyclic. Additionally, one or two ring carbon atoms in the heterocyclyl ring can optionally be replaced with a carbonyl group. When the heterocyclyl ring is partially saturated it can contain one to three ring double bonds provided that the ring is not aromatic.

The terms “alkoxy” refers to the groups of the structure —OR and wherein the group R is independently selected from the alkyl or cycloalkyl groups defined and recited above and hereinafter as appropriate.

The terms “alkylamino” or “dialkylamino” refers to an amino group wherein one or both of the hydrogen atoms are replaced with a group selected from the alkyl or cycloalkyl groups defined and recited above and hereinafter as appropriate.

The terms “halo” and “halogen” refers to fluoro/fluorine, chloro/chlorine, bromo/bromine, or iod/iodine radicals/atoms, relatively. The term “haloalkyl” refers to alkyl and cycloalkyl radicals as defined above, substituted with one or more halogen atoms, including those substituted with different halogens. Exemplary groups are chloromethyl, trifluoromethyl, perfluoropropyl, trichloroethylenyl, chloroacetylenyl, and the like.

In some embodiments of the present invention, the compound is represented by general Formula (IA):

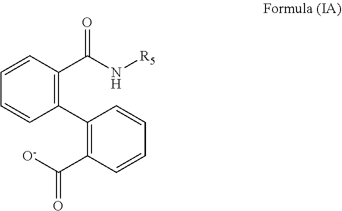

wherein R₅ is the 4-phenylthyazolyl of the following formula:

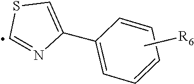

R₆ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, and nitro group.

The exemplary compound of the above embodiment is compound PJL-107:

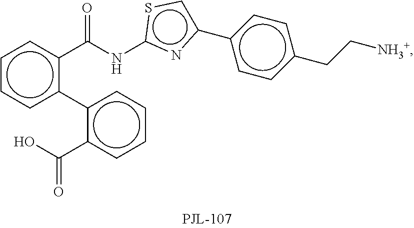

wherein the carboxyl moiety corresponds to the structural determinant referred to as NC1, the benzoic acid benzyl ring moiety corresponds to the structural determinant referred to as HD1 (aromatic rings fall under hydrophobic functionality), the amide moiety corresponds to the structural determinant referred to as HA1, the thiazole moiety corresponds to the structural determinant referred to as AR1, and the anisole moiety corresponds to the structural determinant referred to as HD2 (see, also, Table 3).

In another embodiment, the present invention provides compounds having Formula (IA):

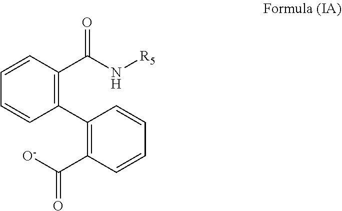

wherein R₅ is the phenyl of the following formula:

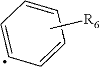

R₆ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, nitro group, and any one of the following radicals:

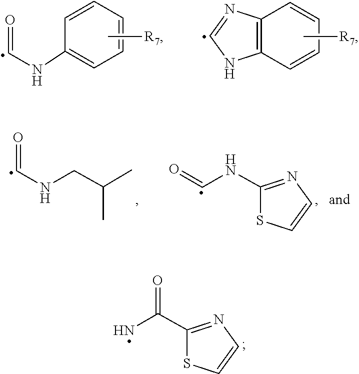

R₇ is attached to any available phenyl ring carbon atom and independently selected from hydrogen, (C₁-C₃)-alkyl, (C₁-C₃)-haloalkyl, (C₁-C₃)-alkoxy, halogen, amino, cyano, hydroxy, and nitro group.

In a particular embodiment, the compounds of the present invention, which are derived from Formula (IA), are represented by general Formula (IA-1):

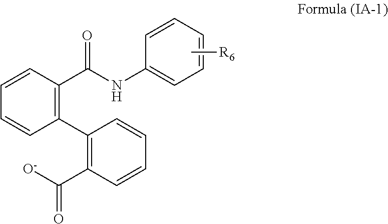

wherein R₆ is attached to any available phenyl ring carbon atom and independently selected from hydrogen, (C₁-C₃)-alkyl, (C₁-C₃)-haloalkyl, (C₁-C₃)-alkoxy, halogen, amino, cyano, hydroxy, nitro group.

In a specific embodiment, the compounds of Formula (IA-1) have the R₆ group defined as hydrogen, halogen, trifluoromethyl, hydroxy or methoxy. The exemplary compounds of this embodiment are:

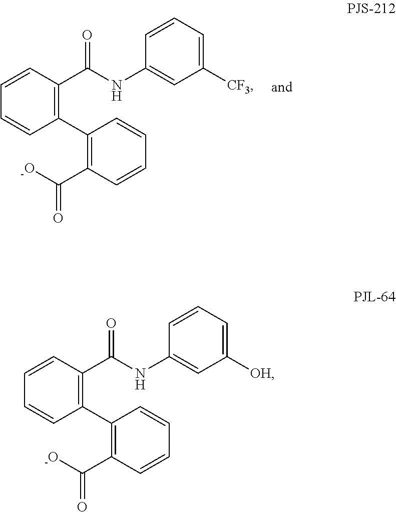

wherein the carboxyl moiety corresponds to the structural determinant referred to as NC1, the benzoic acid aryl ring moiety corresponds to the structural determinant referred to as HD1, the amide moiety corresponds to the structural determinant referred to as HA1, and the —OH/—CF₃ substituted benzene moiety corresponds to the structural determinant referred to as AR1 (see, also, Table 3).

In a further embodiment, the present invention describes the compounds which are derived from Formula (IA) and having Formula (IA-2):

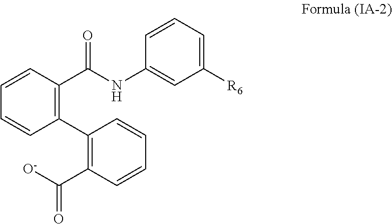

wherein R₆ is selected from the following radicals:

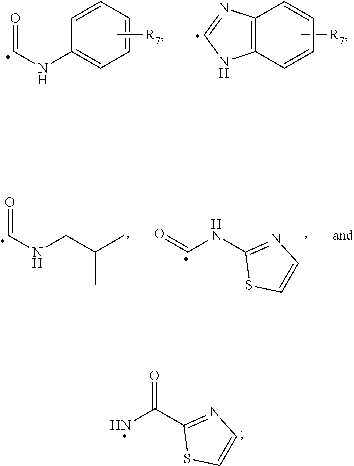

R₇ is attached to any available phenyl ring carbon atom and independently selected from hydrogen, (C₁-C₃)-alkyl, (C₁-C₃)-haloalkyl, (C₁-C₃)-alkoxy, halogen, amino, cyano, hydroxy, and nitro group.

In a specific embodiment, the compounds of Formula (IA-2) have the R₇ group defined as hydrogen, halogen, trifluoromethyl, hydroxy and methoxy. The exemplary compounds of this embodiment are:

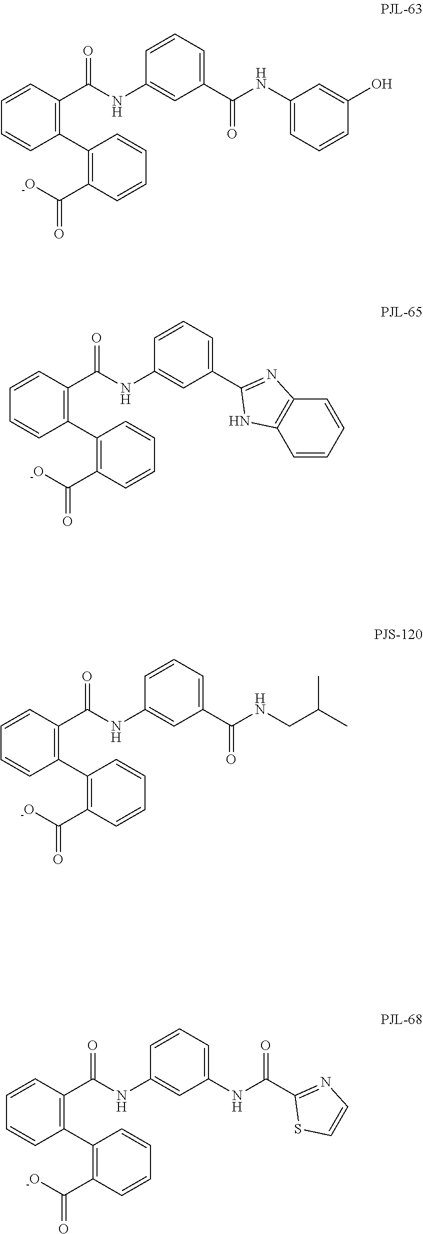

In another embodiment, the compounds of the invention have the following Formula (IB):

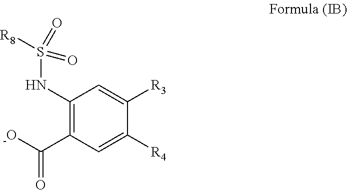

wherein R₃ and R₄ are independently selected from hydrogen, halogen, hydroxy, amino, cyano, (C₁-C₃)-alkyl, (C₁-C₃)-haloalkyl, (C₁-C₃)-alkoxy, and nitro group;
R₈ is selected from any one of the following radicals:

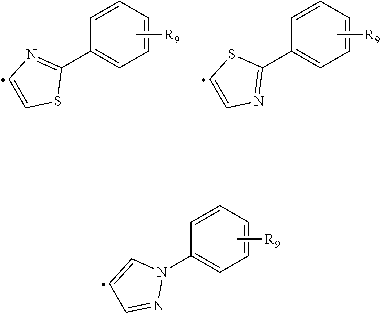

R₉ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, and nitro group.

In a further specific embodiment, the compounds of Formula (IB) have R₃, R₄ and R₉ independently selected from hydrogen, halogen, trifluoromethyl, hydroxy and methoxy.

Exemplary herbicidal compounds of this embodiment are:

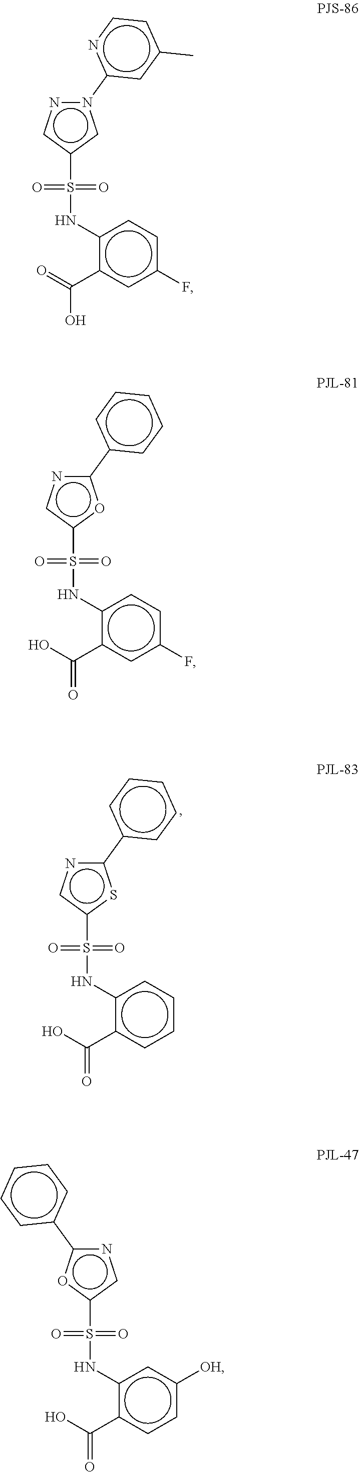

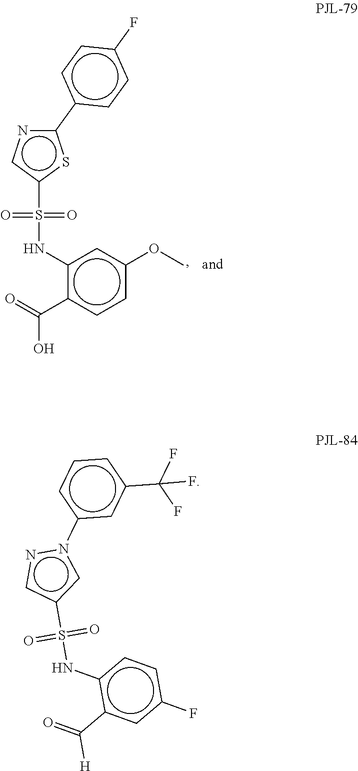

In a further embodiment, the compounds of the invention have the following Formula (IC):

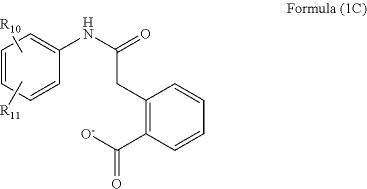

wherein R₁₀ and R₁₁ are attached to any available phenyl ring carbon atom and independently selected from hydrogen, (C₁-C₃)-alkyl, (C₁-C₃)-haloalkyl, (C₁-C₃)-alkoxy, halogen, amino, cyano, hydroxy, nitro group, and any one of the following radicals:

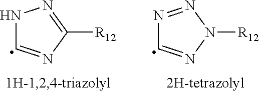

R₁₂ is selected from hydrogen, (C₁-C₃)-alkyl, and (C₁-C₃)-haloalkyl.

In particular embodiment, the compounds of Formula (IC) have R₁₀ and R₁₁ independently selected from hydrogen, hydroxy, and any one of the following radicals:

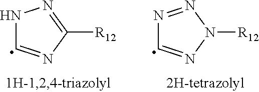

R₁₂ is selected from hydrogen, (C₁-C₃)-alkyl, and (C₁-C₃)-haloalkyl.

In a specific embodiment, the compounds of compounds of Formula (IC) have R₁₀ selected from hydrogen and hydroxy, and R₁₁ is selected from any one of the following radicals:

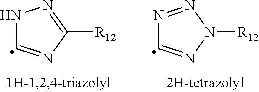

R₁₂ is selected from hydrogen, (C₁-C₃)-alkyl, and (C₁-C₃)-haloalkyl.

In another specific embodiment, the compounds of Formula (IC) have R₁₂ group defined as (C₁-C₃)-alkyl. The exemplary compounds of this embodiment are:

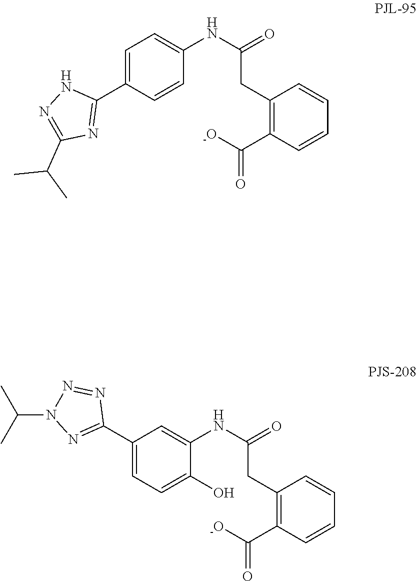

In some embodiments, the compounds of the invention have the following Formula (ID):

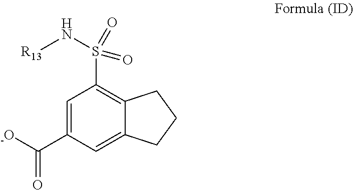

wherein R₁₃ selected from any one of the following radicals:

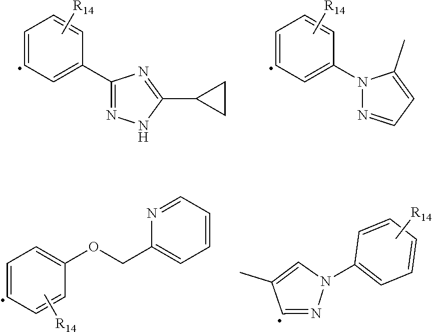

R₁₄ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, nitro group.

In yet further specific embodiment, the compounds of Formula (ID) have R₁₄ selected from hydrogen, halogen, trifluoromethyl, hydroxy and methoxy. The exemplary compounds of this embodiment are:

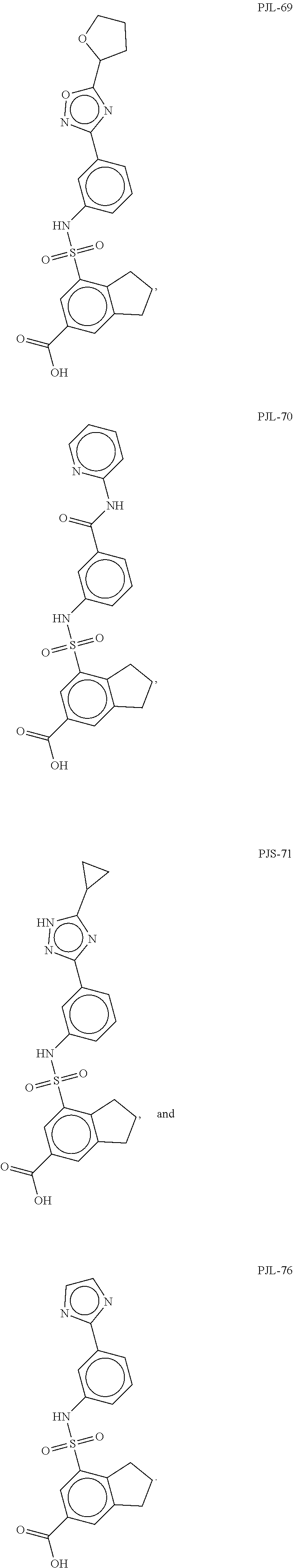

The exemplary herbicidal compound having R₂ defined as the radical of Formula (E) is the compound PJS-211 of the following formula:

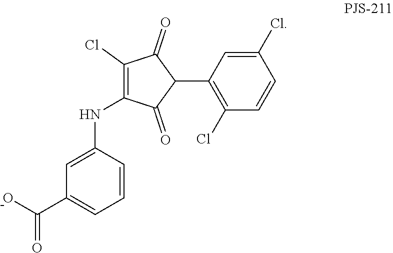

It is to be understood that the scope of the invention encompasses molecules that are known prior to the date of the present invention, but not as herbicides, and further encompasses novel molecules that have been designed, produced and tested for their predicted herbicidal activity. Hence, excluded by proviso from the scope of novel compounds are compounds denoted PJL-2, PJL-5, PJL-21, PJL-22, PJL-27, PJL-29, PJS-30, PJS-31, PJL-39, PJS-39, PJS-41, PJL-43, PJL-59, PJL-60, PJL-61, PJL-62, PJL-63, PJL-64, PJL-65, PJL-66, PJL-67, PJL-68, PJL-88, PJL-91, PJL-92, PJL-93, PJL-95, PJL-96, PJL-103, PJS-110, PJL-120, PJS-120, PJL-128, PJS-208, PJS-211, PJS-212, and PJS-227.

Accordingly, in some embodiments of the present invention, provided herein are novel compounds that were designed based on the identification of OASS as a target for herbicidal compounds, and based on the elucidation of the binding site of SAT in OASS, and based on the physico-chemical properties and structural determinants disclosed herein, and include, without limitation, compounds denoted PJL-47, PJL-69, PJL-70, PJL-71, PJL-74, PJL-76, PJL-79, PJL-81, PJL-82, PJL-83, PJL-84, PJL-85, PJL-86, PJL-106, PJL-107, PJL-108, PJL-109, PJL-110, PJL-111, PJL-114, PJL-115, PJL-116, PJL-117, PJL-118, PJL-119, PJL-121, PJL-122, PJL-123, PJL-125, PJL-126, PJL-127, PJL-129, PJL-130, PJL-131, PJL-132, and PJL-133.

Table 4 presents the herbicidal compounds, according to some embodiments of the present invention, whereas “Scaffold” denotes the correlation of the compound to the scaffold presented in Table 3, and the columns on the right of “Scaffold” present activity assay results (see, Example section that follows below).

TABLE 4

| | | | | | | Root- | Pre-emergence assay | | |
|---|---|---|---|---|---|---|---|---|---|
| | | | | | | growth | | Germina- | Growth |
| Com- | | | ITC | FP % | FP | inhibition | | tion | inhibi- |
| pound | | Scaf- | Kd | inhibi- | IC50 | % | Weed | inhibition | tion |
| Name | Structure | fold | (μM) | tion | (μM) | (50 uM) | model | on (%) | on (%) |
| PJL-107 | 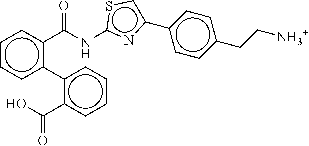 | 1 | 0.32 | 41 | 1.9 | 29 | | | |
| PJL-106 | 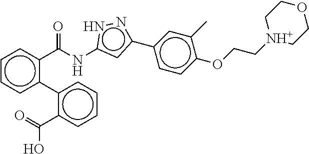 | 1 | 0.94 | 45 | 1.9 | 0 | | | |
| PJL-126 | 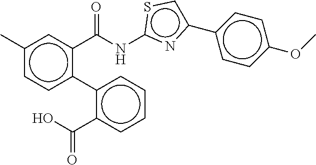 | 1 | 3 | 58 | 2.1 | 93 | | | |
| PJL-110 | 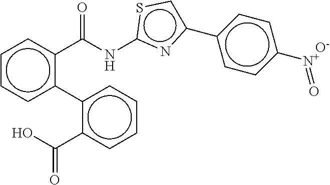 | 1 | n/a | 46 | 2.6 | 98 | | | |
| PJL-125 | 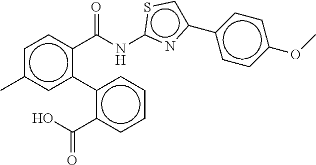 | 1 | n/a | 56 | 3.4 | n/a | | | |
| PJL-130 | 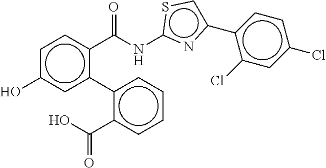 | 1 | n/a | 52 | 3.4 | 93 | | | |
| PJL-116 | 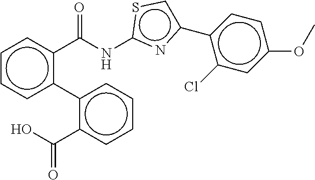 | 1 | 0.5 | 41 | 3.6 | 92 | Trifolium alexandrium | 75 | 80 |
| PJL-65 | 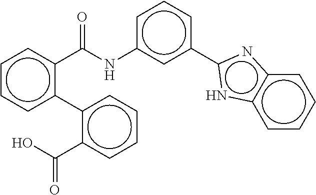 | 1 | 1.77 | 38 | 4 | 0 | | | |
| PJL-132 | 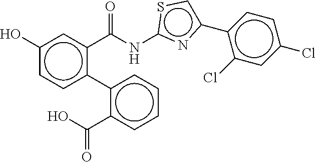 | 1 | n/a | 51 | 4.5 | n/a | | | |
| PJL-61 | 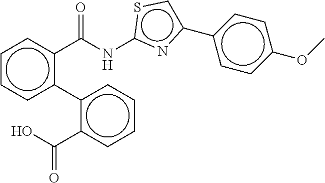 | 1 | 0.4 | 43 | 4.7 | 95 | Trifolium alexandrium Amaranthus palmeri | 70 33 | 60 0 |
| PJL-108 | 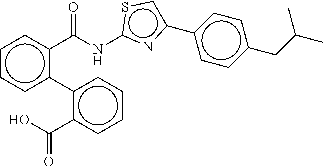 | 1 | n/a | 47 | 4.7 | 17 | | | |
| PJL-60 | 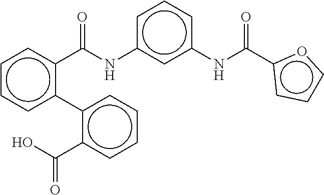 | 1b | 1.27 | 32 | 4.8 | 0 | | | |
| PJL-109 | 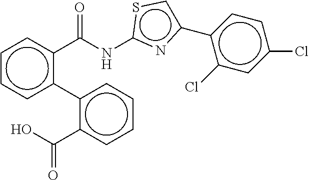 | 1 | 0.07 | 48 | 5.4 | 89 | Solanum nigrum | 63 | 0 |
| PJL-118 | 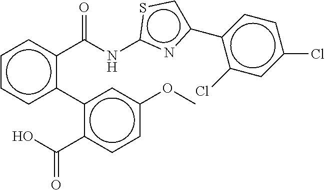 | 1 | 2.3 | 43 | 5.99 | 47 | | | |
| PJL-63 | 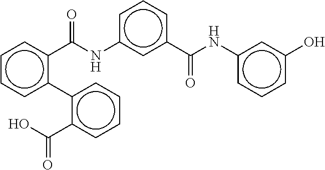 | 1b | 1.48 | 34 | 6.3 | 12 | | | |
| PJL-62 | 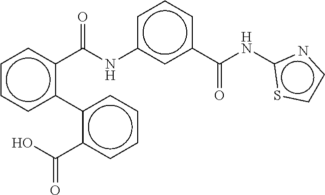 | 1b | 2 | 31 | 6.7 | 0 | | | |
| PJL-133 | 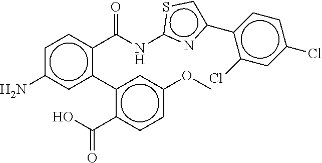 | 1 | n/a | 48 | 7.6 | n/a | | | |
| PJL-119 | 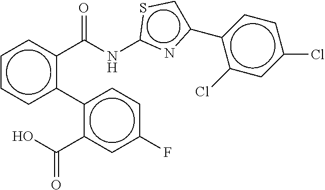 | 1 | n/a | 52 | 8.56 | 100 | | | |
| PJL-131 | 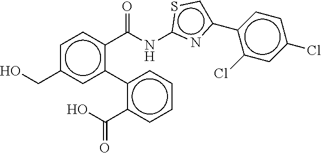 | 1 | n/a | 42 | 9.3 | n/a | | | |
| PJL-68 | 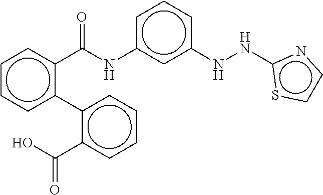 | 1b | 3.4 | 32 | 10 | 0 | | | |
| PJS-212 | 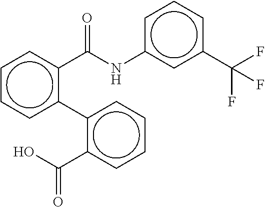 | 1 | 8.11 | 34 | 10.6 | 34 | | | |
| PJL-129 | 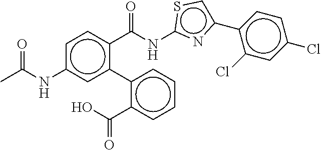 | 1 | n/a | 43 | 11 | | | | |
| PJL-115 | 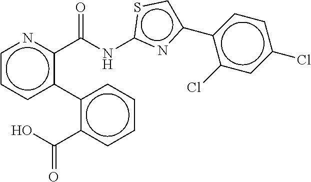 | 1 | n/a | 36 | 12 | 96 | | | |
| PJL-64 | 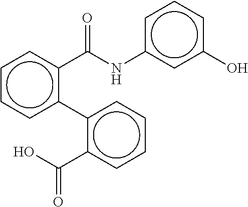 | 1 | 3.4 | 23 | 12.8 | 23 | | | |
| PJL-127 | 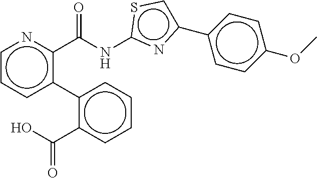 | 1 | 7.6 | 38 | 15.3 | 100 | | | |
| PJL-128 | 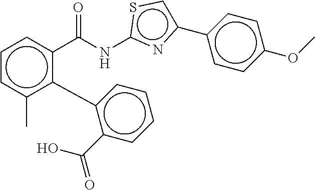 | 1 | n/a | 41 | 18.5 | n/a | | | |
| PJL-67 | 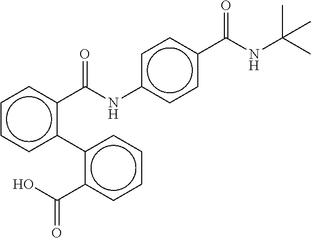 | 1 | 2.38 | 30 | 20.9 | 9 | | | |
| PJL-66 | 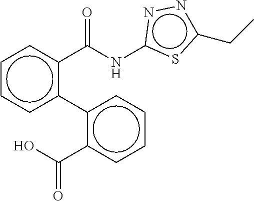 | 1 | 8.6 | 17 | 30.8 | 0 | | | |
| PJL-120 | 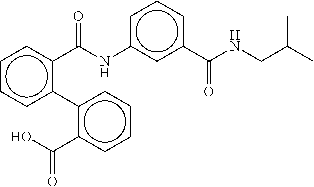 | 1b | 4 | 34 | 34 | 3 | | | |
| PJL-59 | 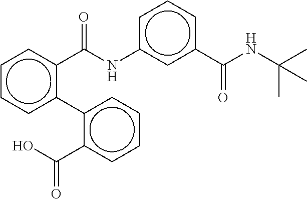 | 1b | n/a | 19 | 58 | 6 | | | |
| PJL-120 | 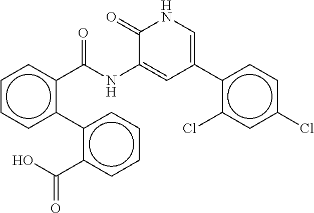 | 1 | n/a | 14 | 115 | 88 | | | |
| PJL-117 | 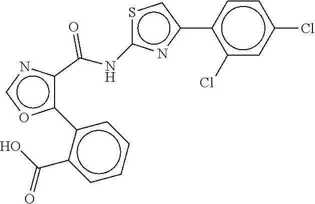 | 1 | n/a | 10 | 248 | 100 | Sinapis arvensis Lolium rigidum | 90 0 | 80 60 |
| PJL-111 | 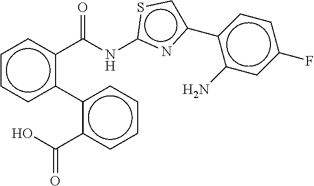 | 1 | n/a | 45 | n/a | 66 | | | |
| PJL-86 | 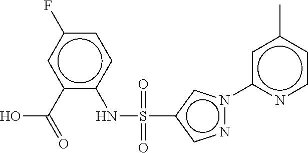 | 2 | n/a | 36 | 20 | 0 | | | |
| PJL-81 | 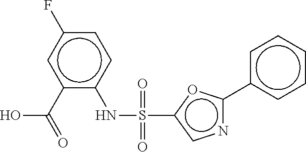 | 2 | 28 | 23 | 32 | 15 | | | |
| PJL-41 | 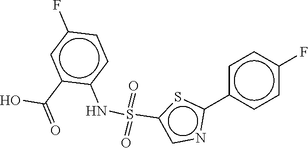 | 2 | 4.8 | 19 | 34 | 85 | | | |
| PJL-114 | 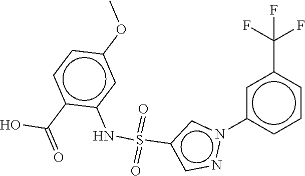 | 2 | 12 | 17 | 41 | | | | |
| PJL-84 | 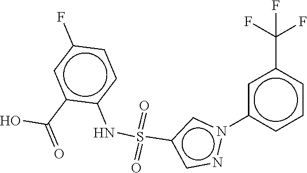 | 2 | 10.9 | 24 | 44 | 47 | | | |
| PJL-47 | 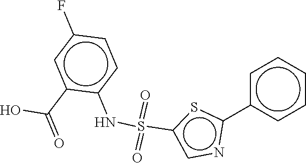 | 2 | 6 | 16 | 68 | 88 | Amaranthus palmeri | 66 | 20 |
| PJL-79 | 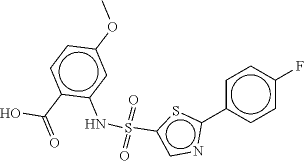 | 2 | 7.1 | 18 | 55 | 97 | Sinapis arvensis | 95 | 90 |
| PJL-83 | 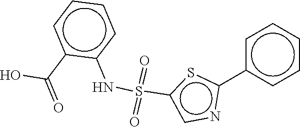 | 2 | 9.9 | 17 | 74 | 0 | | | |
| PJL-85 | 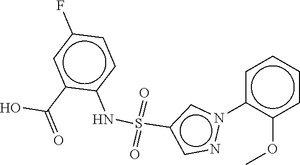 | 2 | n/a | 15 | 111 | 0 | | | |
| PJL-5 | 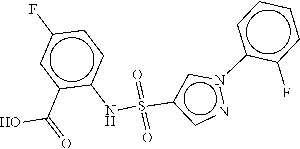 | 2 | n/a | 16 | 148 | 56 | | | |
| PJL-82 | 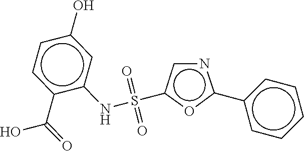 | 2 | n/a | 11 | 150 | 0 | | | |
| PJL-27 | 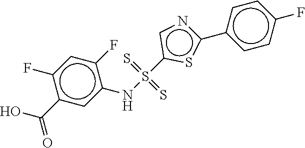 | 2/3 | n/a | 11 | 188 | 25 | | | |
| PJL-110 | 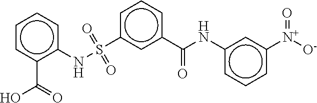 | 2 | n/a | 7 | 315 | 12 | | | |
| PJL-121 | 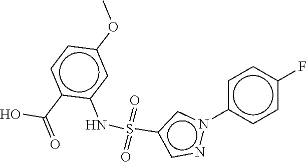 | 2 | n/a | 4.6 | 322 | 72 | | | |
| PJL-122 | 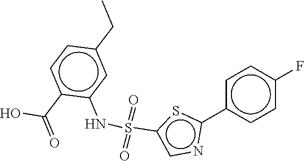 | 2 | n/a | 25 | n/a | | | | |
| PJL-123 | 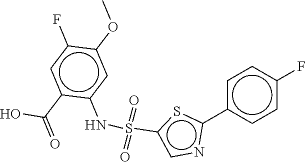 | 2 | n/a | 16 | n/a | n/a | | | |
| PJL-71 | 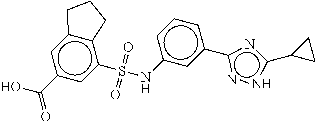 | 3 | 6.11 | 24 | 21 | 17 | | | |
| PJL-74 | 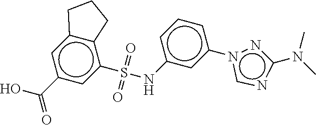 | 3 | n/a | 26 | 22 | 9 | | | |
| PJS-227 | 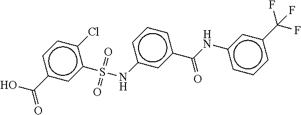 | 3 | n/a | 18 | 54.2 | 39 | | | |
| PJS-39 | 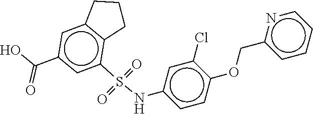 | 3 | 7.5 | 14 | 72 | 2.3 | | | |
| PJL-43 | 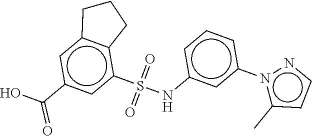 | 3 | 9.9 | 17 | 87 | 6 | | | |
| PJL-39 | 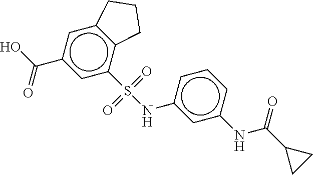 | 3 | 10.1 | 12 | 127 | 18 | | | |
| PJL-29 | 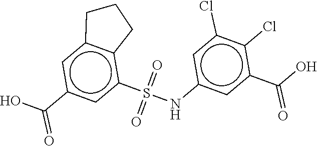 | 3 | 3.5 | 13 | 132 | 11 | | | |
| PJL-21 | 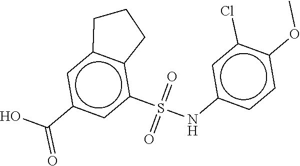 | 3 | n/a | 14 | 149 | 0 | | | |
| PJL-22 | 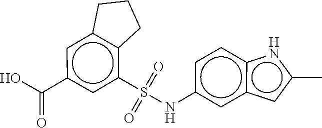 | 3 | n/a | 13 | 206 | 0 | | | |
| PJS-31 | 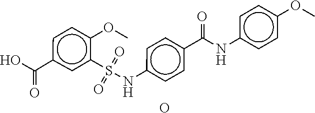 | 3 | 5.6 | 11 | 225 | 0 | | | |
| PJL-2 | 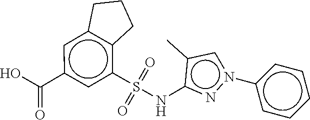 | 3 | n/a | 11 | 236 | 70 | | | |
| PJL-30 | 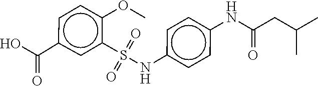 | 3 | 6.6 | 11 | 300 | 0 | | | |
| PJL-76 | 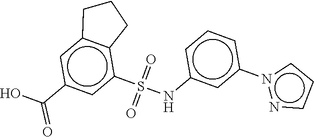 | 3 | 18.3 | 16 | n/a | 0 | | | |
| PJL-70 | 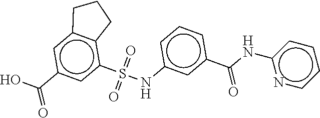 | 3 | n/a | 15 | n/a | 0 | | | |
| PJL-69 | 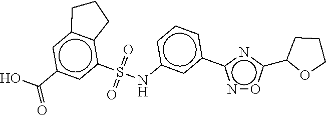 | 3 | 8.12 | 21 | n/a | n/a | | | |
| PJL-211 | 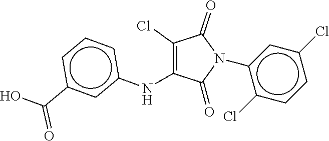 | 4 | 11.6 | 17 | 63.9 | 72 | | | |
| PJL-103 | 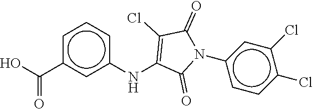 | 4 | 9.37 | 21 | 73 | 0 | | | |
| PJL-95 | 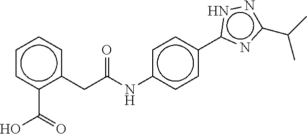 | 5 | n/a | 27 | 22 | 10 | | | |
| PJL-96 | 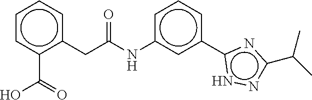 | 5 | n/a | 22 | 43 | 2 | | | |
| PJL-91 | 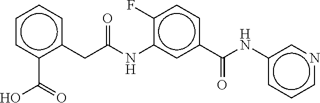 | 5 | n/a | 13 | 55 | n/a | | | |
| PJL-88 | 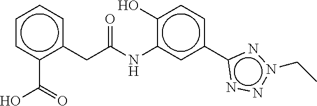 | 5 | n/a | 10 | 80 | 0 | | | |
| PJL-208 | 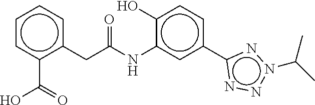 | 5 | 19.8 | 16 | 97 | 0 | | | |
| PJL-92 | 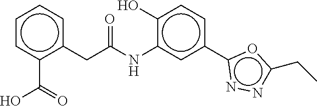 | 5 | n/a | 13 | 122 | 0 | | | |
| PJL-93 | 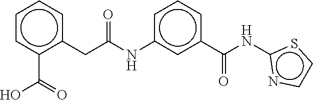 | 5 | 5.3 | 21 | n/a | 0 | | | |

According to some embodiments of the present invention, the compounds provided herein are contemplated in a form of a pro-herbicide, a solvate, a hydrate and/or agronomically acceptable salt thereof.

The phrase “agronomically acceptable salt” refers to a charged species of the parent herbicidal compound provided herein, and its counter ion, which is typically used to modify the solubility characteristics of the parent compound and/or to reduce any significant adverse effect by the parent herbicidal compound, while not abrogating the biological activity and properties of the administered herbicidal compound. An example, without limitation, of an agronomically acceptable salt would be a carboxylate anion and a cation such as, but not limited to, ammonium, sodium, potassium and the like.

The term “pro-herbicide” refers to a compound that is converted into the active herbicide under the condition it is used at, in the context of an herbicidal composition or in planta. A pro-herbicide is typically useful for facilitating the application of the herbicide or to improve its absorption into the plant cell. The herbicide may, for instance, be insoluble in a particular carrier, or its bioavailability too low, whereas the pro-herbicide is more soluble or more bioavailable for absorption into a plant. Pro-herbicides are also often used to achieve a sustained release of the active compound. An example, without limitation, of a pro-herbicide would be a compound of the present invention, having one or more carboxylic acid moieties, which is administered as an ester (the “prodrug”), e.g., methyl ester or ethyl ester. Such a pro-herbicide readily hydrolyzes, to thereby provide the free compound (the parent herbicide). The selected ester may affect both the solubility characteristics and the hydrolysis rate of the prodrug.

According to some embodiments of the present invention, the herbicide compounds provided herein are in a form of a salt, preferably an agronomically acceptable salt, or in the form of an ester (i.e., a pro-herbicide), preferably a methyl ester. Further preferably the herbicide compounds provided herein are in a form of a salt or an ester of the carboxy group on the benzoic acid moiety thereof. Formula (I*) and Formula (I**) illustrate an exemplary ammonium salt and a methyl benzoate ester of the compound of Formula (I), respectively:

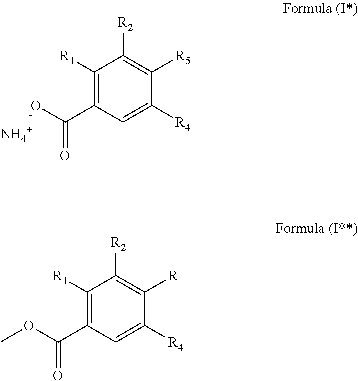

The term “solvate” refers to a complex of variable stoichiometry (e.g., di-, tri-, tetra-, penta-hexa-, and so on), which is formed by a solute (the polymer as described herein) and a solvent, whereby the solvent does not interfere with the biological activity of the solute. Suitable solvents include, for example, ethanol, acetic acid and the like. The term “hydrate” refers to a solvate, as defined hereinabove, where the solvent is water.

### Uses of the Compounds:

The compounds provided herein have been developed and designed to exhibit herbicidal activity, namely to be used as herbicides that adversely affect plant growth. As known in the art, an herbicide is a compound which can penetrate into at least one plant cell and adversely control or modify the plant's growth, e.g., killing, retarding, defoliating, desiccating, regulating, stunting, tillering, stimulating and dwarfing. The term “plant” refers to all physical parts of a plant, including cells, organelles, seeds, seedlings, saplings, roots, tubers, stems, stalks, foliage, and fruits. The term “growth” in the context of plants includes all phases of development from seed germination to natural or induced cessation of life.

Since the compounds of the present invention have been designed to modulate the OASS-SAT PPI, these compounds can enter a plant cell, and specifically penetrate the plant organelle where OASS is found. The compounds of the present invention can be used for seed or foliage treatment. In a particular embodiment, the compounds of the present invention are used as selective herbicides, non-selective herbicides, agricultural herbicides, non-agricultural herbicides, herbicides in integrated pest management, herbicides in gardening, herbicides in clearing waste ground, herbicides in clearing industrial or constructions sites, or herbicides in clearing roadsides, railways and railway embankments.

As will be demonstrated below, the compounds of the present invention are indeed herbicidally active and would be effective in regulating growth of a wide variety of undesirable plants, i.e., weeds, when applied, in herbicidally effective amount, to the growth medium prior to emergence of the weeds or to the weeds subsequent to emergence from the growth medium. The term “herbicidally effective amount” is that amount of a compound or mixture of compounds of the present invention required to so injure or damage weeds such that the weeds are incapable of recovering following application. Alternatively, the term “herbicidally effective amount” describes an amount of an herbicide compound which adversely controls or modifies plant growth.

The actual amount used depends upon several considerations, including weather, soil, neighboring crops, particular weed susceptibility, and overall cost limitations. The quantity of a compound or mixture of compounds of the present invention applied in order to exhibit a satisfactory herbicidal effect may vary over a wide range and depends on a variety of factors, such as, for example, hardiness of a particular weed species, extent of weed infestation, climatic conditions, soil conditions, method of application and the like. Of course, the efficacy of a particular compound against a particular weed species may readily be determined by routine laboratory or field testing in a manner well known to the art.

Weeds that may be effectively controlled by the application of compounds of the present invention are for example, barnyard grass (Echinochloa crus-galli), crabgrass (Digitaria sauguinalis), hemp sesbania (coffeeweed) (Sesbania herbacea), jimsonweed (Datura stramonium), Johnson grass (Sorghum halepense), tall morning glory (Ipomoea purpurea), wild mustard (Brassica rapa), prickly sida (Sida spinosa), velvetleaf (Abutilon theophrasti), wild oat (Avena fatua), yellow foxtail (Setaria glauca), yellow nutsedge (Cyperus esculentus) and the like.

### Herbicidal Compositions:

As mentioned above, the compounds of the present invention exhibit herbicidal activity, at least in the sense of inhibiting or disrupting the cysteine biosynthesis pathway, or by inhibiting the association of OASS and SAT. These compounds, either previously disclosed but not as herbicides, or novel and designed based on the provisions of the present invention, constitute an active ingredient in an herbicidal composition. The compositions are designed to facilitate the application of the compounds by rendering the compound into a colloid (emulsion of suspension), into granules or into a powder. The formulation also assists in the entry of the compound into the cells of the plant. Hence, according to an aspect of some embodiments of the present invention, there is provided an herbicidal composition that includes as an active ingredient, any one or more of the compounds provided herein, and an agronomically acceptable carrier and optionally at least one adjuvant, wherein the carrier or the optional adjuvant are for allowing the compound to be used as herbicide.

The herbicidal compositions are applied to the terrestrial area of the unwanted vegetation, to the undesired plants or to a habitat thereof, in a form of an herbicidal composition comprising a herbicidally effective amount of the compound or compounds provided herein. As a post-emergent, the herbicides of the invention may be applied to the terrestrial area of the unwanted vegetation neat or as an emulsion or solution. The effective amount will differ between compounds, undesired vegetation, soils, climate, season, and other per-case conditions. A person skilled in the field of herbicides is capable of determining the herbicidal effective amount by conducting a simple concentration sensitivity test, as typically done in the art per-case. The Examples section that follows below provides at least two procedures that can be used to determine the herbicidal effective amount of an herbicide.

A compound or compounds of the present invention can be used in various formulations with agronomically acceptable inert carriers, solvents, adjuvants, other herbicides, or other commonly used agricultural compounds, for example, insecticides, fungicides, pesticides, fertilizers or the like. The compounds provided herein can be found in a composition with an agronomically acceptable carrier that allows the compound to be used as an herbicide. When formulated with other typically used agronomically acceptable materials, the amount of compound or compounds of this invention may vary over a wide range, for example, from about 0.05 to about 95 percent by weight on weight of the formulation. Typically, such formulations would contain from about 5 to 75 percent by weight of a compound or compounds of the present invention.

A typical herbicidal composition includes, beside of the active ingredient (the herbicidal compound or agent), carriers/solvents, diluents, extenders, synergists, slow-release agents, surfactants, colloid stabilizers, dispersing agents, emulsifying agents, safeners, pH adjusters, conditioning agents, wetting agents, acidifiers, buffering agents, water conditioners, anti-foaming agents, antifreeze agents, biocides, compatibility agents, drift control agents, and any suitable combination of these adjuvants, as well as colorants and odorants. According to some embodiments of the present invention, at least one adjuvant is selected to facilitates the entry of the compound to at least one type of plant cell.

The compounds of the present invention alone or in the form of an herbicidal composition, formulated with other agronomically acceptable materials, are typically applied in the form of dusts, granules, wettable powders, solutions, suspensions, aerosols, aqueous colloids, emulsions, dispersions or the like in a manner well known to the art.

Any solvent in which the herbicide is soluble, or may be emulsified or suspended in, may be employed as a carrier/diluent. Suitable solvents include water or water-soluble alcohols, such as methanol, ethanol, and isopropyl alcohol, or a ketone such as acetone or methyl ethyl ketone. Such compounds further form emulsions and suspensions with/in water.

The herbicidal composition provided herein may include one or more safeners, which are chemical agents used in combination with herbicides to make them “safer”, namely, to reduce the effect of the herbicide on crop plants, and to improve selectivity between crop plants vs. weed species being targeted by the herbicide. Herbicide safeners can be used to pretreat crop seeds prior to planting, or they can be sprayed on plants as a mixture with the herbicide. In the context of some embodiments of the present invention, a safener can be one or more of benoxacor, BPCMS, cloquintocet, cyometrinil, cyprosulfamide, dichlormid, dicyclonon, dietholate, fenchlorazole, fenclorim, flurazole, fluxofenim, furilazole, isoxadifen, jiecaowan, jiecaoxi, mefenpyr, mephenate, metcamifen, naphthalic anhydride, and oxabetrinil.

Adjuvants are spray solution additives that are mixed with an herbicide composition to improve performance of the spray mixture. Adjuvants can either enhance activity of an herbicide's active ingredient (activator adjuvant) or offset any problems associated with spray application, such as adverse water quality or wind (special purpose or utility modifiers). In the context of embodiments of the present invention, herbicidal composition adjuvants include surfactants (nonionic, ionic and amphoteric), wetting agents (spreaders), penetrants, oil adjuvants (COC; petroleum oils and vegetable oils), ammonium fertilizers, utility adjuvants, dyes, drift control/foaming agents, thickening agents, deposition agents (stickers), water conditioners, compatibility agents, pH buffers, humectants, defoaming/antifoam agents and UV absorbents.

Surfactants, or surface-active agents, are a broad category of activator adjuvants that facilitate and enhance the absorbing, emulsifying, dispersing, spreading, sticking, wetting, or penetrating properties of herbicides. Wetter/Spreaders are most often used with herbicides to help it spread over and penetrate the waxy cuticle (outer layer) of a leaf or to penetrate through the small hairs present on the leaf surface. Because of the high surface tension of water, spray mixture droplets can maintain their roundness and sit on the leaf hairs or waxy surface without much of the herbicide actually contacting the leaf. The primary purpose of a wetter/spreader is to reduce the surface tension of the spray solution to allow more intimate contact between the spray droplet and the plant surface. They may also act to change the permeability of the leaf surface. Most wetter/spreaders used with herbicides are considered non-ionic surfactants. This means that these compounds have no electrical charge and are compatible with most pesticides. There are cationic (positive charge) and anionic (negative charge) surfactants, but they are not as commonly used, with the exception of the cationic surfactant in the Roundup® formulation of glyphosate. Wetter/spreaders have the physical characteristics of both oil and water (amphiphilic). There are several different basic chemistries of wetter/spreaders, including, without limitation, cationic wetter/spreaders such as ethoxylated fatty amines (e.g., polyethoxylated amine; POEA), cationic wetter/spreaders, and non-ionic wetter/spreaders such as alkylphenol ethoxylate-based. Commercially available surfactant, suitable for use in the context of an herbicidal composition, include, without limitation, AirForce®+UAN, Cadence®, DyneAmic®, Dyne-A-Pak®, Freeway®, Inergy, Invade Xtra®, Kinetic HV®, Persist® Advanced+UAN, Phase®/Phase II, Regiwet, Renegade®, Rivet®, Silkin®, Soysurf Plus, Soysurf Xtra, Syl-tac®, Triple Play, and Volare™+UAN.

Additional information, examples and use guides can be found in the literature, such as, for example, “Compendium of Herbicide Adjuvants”, 2016, 13^{th} Edition, by Bryan G. Young, Purdue University, Joseph L. Matthews, Southern Illinois University, and Fred Whitford, Purdue Pesticide Programs, or in Young, Bryan G.; Joseph L. Matthews, “Herbicide adjuvants”, Encyclopedia of Agrochemicals, 2003, 707-718; and Tu, M., and J. M. Randall. “Adjuvants”, TU, M. et al., Weed Control Methods Handbook the Nature Conservancy, Davis: TNC (2003): 1-24.

Oil adjuvants can increase the penetration of oil-soluble herbicides into plants, and are commonly used when conditions are hot and dry, and/or when leaf cuticles are thick. They are derived from either refined petroleum (mineral) oils or from vegetable oils (including seed oils), and do not readily mix with water. Therefore, when an oil adjuvant is combined with water in a spray tank, a surfactant emulsifier must also be added, which distributes the oil droplets (micelles) uniformly throughout the mix. These “emulsifiable oil” adjuvant combinations typically contain both a non-phytotoxic oil (typically ranging 80 to 99%) and a surfactant (1 to 20%), and are added to the spray tank usually as just 1% of the total spray volume. Emulsifiable oil adjuvant blends can enhance the absorption of an oil-soluble herbicide into the plant more than an oil adjuvant by itself. Adding a surfactant to the mixture not only emulsifies the oil in the water-based spray solution, but also lowers the surface tension of the spray solution. These adjuvants can also increase herbicide absorption through the plant cuticle, increase spray retention on leaf surfaces, and reduce the time needed for the herbicide composition to become rainfast. The exact mode of action of these oils is unknown, but they enhance the spread of droplets on plant surfaces. They may also split open the cuticle and increase both the fluidity of cuticular components and herbicide diffusion rates. Two types of emulsifiable oil adjuvants are “crop oils” and “crop oil concentrates” (COC).

Utility adjuvants are added to improve the application of the formulation to the target plants. By themselves, utility adjuvants do not directly enhance herbicidal activity. Instead, they change the physical or chemical properties of the herbicidal composition in ways that make it easier to apply to the target plant(s), minimize unwanted effects, and broaden the range of conditions under which a given herbicide formulation can be effective. Most utility adjuvants are typically not used in wildland situations, since herbicides applied in wildlands are generally not applied aerially, with large booms, or in tank mixtures with several herbicides and other additives. Some activator adjuvants are also utility adjuvants and some even have herbicidal effects of their own.

Wetting agents or spreading agents lower surface tension in the spray droplet, and allow the herbicide formulation to form a large, thin layer on the leaves and stems of the target plant. Since these agents are typically nonionic surfactants diluted with water, alcohol, or glycols, they may also function as activator adjuvants (surfactants). However, some wetting or spreading agents affect only the physical properties of the spray droplets, and do not affect the behavior of the formulation once it is in contact with plants.

Drift control agents are designed to reduce spray drift, which most often results when fine (<150 μm diameter) spray droplets are carried away from the target area by breezes, including those caused by the aircraft or vehicle carrying the spray equipment. Drift control agents alter the viscoelastic properties of the spray solution, yielding a coarser spray with greater mean droplet sizes and weights, and minimizing the number of small, easily-windborne droplets. These agents are typically composed of large polymers such as polyacrylamides, and polysaccharides, and certain types of gums. Foaming agents also act as drift control agents. When used with specialized nozzles, these agents create foams with different degrees of stability. These foams can be placed more precisely than standard liquid sprays, and are sometimes used to mark the edge of spray applications. Foams ensure complete coverage without over-spraying. Foaming agents are usually added in quantities of 0.1 to 4.0% of the entire herbicidal composition.

Thickening agents and other viscosity modifiers can modify the viscosity of the herbicidal composition and are used to reduce drift, particularly for aerial applications, and primarily in agriculture. Thickening agents may include water swellable polymers that can produce a “particulated solution,” hydroxyethyl celluloses, and/or polysaccharide gums. Viscosity can also be increased by making invert emulsions (follow directions on individual herbicide labels) of the herbicidal composition. The compatibility of the thickening agent with the herbicidal composition can be influenced by the order of mixing, pH, temperature, and/or the salt content of the herbicidal composition. Thickening agents are typically used in areas where sensitive populations or crops are growing close to treated areas.

Deposition agents, or stickers, are used to reduce losses of formulation from the target plants due to the droplets evaporating from the target surface, or beading-up and falling off. Spray retention on difficult-to-wet leaf surfaces is regulated by the degree of surface tension and energy dissipation during the spray process. Deposition agents such as guar gum can reduce surface tension while increasing the viscoelasticity of the droplets. Stickers keep the herbicide in contact with plant tissues by remaining viscous, and therefore resist being washed-off by rain or knocked off by physical contact. Stickers are generally the most useful with dry wettable powder and granule formulations. Film-forming vegetable gels, emulsifiable resins, emulsifiable mineral oils, vegetable oils, waxes, and water-soluble polymers can all be used as stickers. Fatty acids (technically anionic surfactants) are frequently used as stickers, and although they are “naturally derived” and are typically considered safe, they may have considerable contact activity. Certain low volatility oils may also function as stickers.

Water conditioners are frequently added to herbicidal compositions when the water used in the formulation is high in salts in order to minimize or prevent reactions between ions, which would result in the formation of precipitates of insoluble salts. When there are many cations present, as in hard water, they can react with the herbicide, decreasing the uptake and effect of the herbicide. For instance, high levels of calcium in water (hard water) reduce the control efficacy of glyphosate. Similarly, sodium bicarbonate reduces the efficacy of sethoxydim. A water conditioner, such as ammonium sulfate (which also happens to be a nitrogen fertilizer), can negate this effect.

Compatibility agents prevent chemical and/or physical interactions between different herbicides and fertilizers that could lead to non-homogeneous or unsprayable mixtures when these compounds are combined. In most cases, the herbicide label will state which herbicides may or may not be mixed together. Some herbicides are applied with fertilizers or fertilizer solutions, especially in agricultural settings, and compatibility agents are used to keep these herbicides in suspension, and are generally added with a liquid fertilizer. Most herbicides can be applied in nitrogen solutions without any compatibility problems, but compatibility may be poor when the water contains high levels of various salts (hard water), or when the water is unusually cool.

Defoaming and antifoam agents reduce or suppress the formation of foam in spray tanks. Many herbicidal compositions have a tendency to foam excessively, especially when mixed with soft water, which can cause problems during mixing (foam overfill) or when rinsing the sprayer. Most defoamer agents are dimethopolysiloxane-based, but silica, alcohol, and oils have also been used for this purpose. Defoaming agents can reduce surface tension, physically burst the air bubbles, and/or otherwise weaken the foam structure. In general, it is easier to prevent foam formation than to eliminate foam after it forms. Antifoam agents are usually dispensed from aerosol cans or plastic-squeeze bottles, and are added directly to the herbicidal composition at the onset of foam formation. The highest concentration needed for eliminating foam is typically about 0.1% of the herbicidal composition. Some applicators in agricultural settings even use kerosene or diesel fuel at about 0.1% for eliminating foam in spray tanks, but this is not recommended in natural areas.

Natural sunlight, especially ultraviolet light, may degrade some herbicides. A few adjuvants that protect herbicides from the deleterious effect(s) of sunlight are available. They may do this by either physical or chemical processes, such as by increasing the rate of herbicide uptake into the cuticle, or by absorbing the UV-light themselves.

Dyes are commonly used for spot or boom spraying. It is generally recommended that the use of a dye for most herbicide treatments in wildlands even if applied with small handheld sprayers or wicks because the presence of a dye makes it far easier to see where the herbicide has been applied and where it has dripped, spilled or leaked. Dyes make it easier to detect missed spots and to avoid spraying a plant or area twice. It is never appropriate to use food coloring or any other substances that have not been approved or labeled by the authorities for use as herbicide adjuvants.

According to some embodiments of the present invention, the compound provided herein can be formulated into several types of herbicidal compositions, which include, without limitation:
EC—emulsifiable concentrate;
DC—dispersible concentrates;
EW—emulsion in water;
ME—microemulsion;
SC—aqueous suspension concentrates;
SE—aqueous suspo-emulsion;
OD—oil dispersion; and
WG—water dispersible granule.

A suspo-emulsion (SE) is a formulation containing both solid and liquid (or low melting point solid plus solvent) active ingredients dispersed in an aqueous phase. Suspo-emulsions allow to utilize together in the same water-based formulation different active ingredients with different physical-chemical characteristics.

These formulations present systems that can be diluted in water, which is the most common way to use herbicides. Some of the formulations are in the form of colloids (emulsions of liquids and/or suspensions or dispersions of solids) for cases that complete dissolving of the herbicidal compound cannot be achieved in a single or mixed solvent.

These formulations also relate to the particle size and the concentration of the active compound in the final product, and therefore a relatively wide range of solutions are provided.

Table 4 below includes, without limitation, the vast majority of practical operational and cost-effective herbicidal compositions which are encompassed and contemplated as embodiments of the present invention. Depending on the type of formulation, the concentration of the listed components also varies (Table 4 presents the amount of the ingredient in weight percent of the total weight of the composition).

TABLE 5

| | Amount of | Amount of | Amount of | Amount of | |
|---|---|---|---|---|---|
| Formulation | Active | organic | non-ionic | anionic | Amount |
| type | ingredient | solvent | surfactant | surfactant | of water |
| EC | 5-60 | Up to 100 | 2-8 | 2-5 | — |
| DC | 5-25 | Up to 100 | 1-10 | 1-10 | — |
| EW | 3-30 | 30-70 | 3-10 | 3-10 | 30-70 |
| ME | 3-40 | 4-45 | 10-30 | 10-30 | Up to 100 |
| SC | 20-70 | — | 0.5-5 | 0.5-5 | Up to 100 |
| SE | 10-50 | 5-35 | 2-8 | 3-8 | 5-35 |
| OD | 3-40 | Up to 100 | 3-8 | 3-8 | — |
| WG | 30-90 | — | 1-25 | 1-20 | — |

Solvents and carriers include, without limitation, mineral oils, vegetables oils (e.g., castor oil, linseed oil, rapeseed oil and derivatives thereof, cotton oil, palm oil, soyabean oil, sunflower oil, sesame seed oil and the line), ketones (e.g., butyl derivatives ketones, mainly isobutyl's), alcohols (e.g., propanols, butanols, pentanols, hexanols, glycols, and the likes), aliphatic ethers and esters, C3-C7 acetates, acids (e.g., propanoic acids, butanoic acids, pentanoic acids, haxanoic acids and the like).

Anionic surfactants deprotonate at neutral and acid pH values and form micelles at low concentrations. They have strong wetting and emulsifying capabilities. Anionic surfactants suitable for use in the herein provided herbicidal compositions include, without limitation, sulfonates (e.g., alkyl benzene sulfonates salts, alkyldiphenyl disulphonate oxides, and sodium sulfonate olefines), sulfosuccinate (e.g., dioctyl sulfosuccinates), sulfate esters, phosphate esters, phosphate ethers, ether carboxylates, acrylics, sodium salts, amine salts, lignin-based salts, fatty acid derivatives, and various naphthalenes.

The main role of a non-ionic surfactants is to emulsify oil-in-water in the spray tank as well as in can formulation stabilization. Non-ionic surfactants provide steric stabilization and therefore improves stability. Non-ionic surfactants are frequently used in combinations, which include a low polarity partner and a high polarity partner to balance the polarity requirements of the oil phase as well as low HLB partner with high HLB partner. Non-ionic surfactants include, without limitation, synthetic alcohol alkoxylates ethoxylates, natural alcohol ethoxylates, alkyl phenol ethoxylates, alkylpolysaccarides, amine ethoxylates, ethylene oxide-propylene oxide block polymers, fatty acid esters ethoxylates, fatty amine ethoxylates, vegetable oil ethoxylates, polymeric options, and combinations thereof.

### Herbicidal Methods:

Since some of the aspects of the present invention are based on the identification of OASS as a plant protein target for herbicidal activity, one of the aspects of the present invention is the provision of OASS as an herbicidal target. Furthermore, since some of the aspects of the present invention is the provision of a small molecule that is defined in terms of structural determinants, physico-chemical properties and activity, so as to bind to OASS at the binding site of SAT, there is provided a method for inhibiting the binding of SAT to OASS, including uncoupling the proteins from each other once bound, which his effected by contacting OASS with any one of the compounds provided herein. According to an aspect of some embodiments of the present invention, inhibiting the binding of SAT to OASS causes plant growth inhibition, namely inhibiting the binding of SAT to OASS has herbicidal consequences. According to embodiments of the present invention, the compound provided herein inhibits the binding of SAT to OASS since it exhibits functional groups at position and orientation so as to bind to OASS at high affinity. This affinity is afforded due to interaction of the compound with at least three, at least four or all five groups of amino acid residues of OASS. The groups of amino acid residues in OASS, numbered according to the numbering convention used in PDB ID: 2ISQ, are selected from the group consisting of:
F148, G181 and/or A228;
F148 and/or M125;
K46, T78 and/or Q147;
S75, T74 and/or Q147; and
I129, F148 and/or F230.

In some embodiments, the groups of amino acid residues in OASS with which the compound interacts, are selected from the group consisting of:
F148, G181 and A228;
F148 and M125;
K46, T78 or Q147;
S75 or T74 or Q147; and
I129, F148 and F230.

In a further embodiment of the present invention, a method for the control of undesired vegetation or clearing areas from the undesired vegetation comprises applying to the terrestrial area of said undesired vegetation a herbicidally effective amount of a compound or compounds of the present invention. The method of the invention may be used to control established vegetation in the vicinity of a seeded crop or in a weed concentrate area by contacting the foliage of the unwanted vegetation with the herbicidal composition. The herbicidal activity of such herbicidal compositions rapidly dissipates in the unwanted vegetation upon contact. The terrestrial area of the undesired plants treated by the compounds of the present invention may be agricultural areas, crop fields, gardens, waste grounds, industrial or constructions sites, railways or railway embankments, waterways, canals and ponds (to control water weeds and algae).

According to an aspect of some embodiments of the present invention, there is provided a method of inhibiting the post-emergence growth of a plant (e.g., undesired vegetation), which is effected by contacting any part of the plant with a herbicidally effective amount of any one of the compounds or compositions provided herein.

According to an aspect of some embodiments of the present invention, there is provided a method of inhibiting the pre-emergence growth of a plant (e.g., undesired vegetation), which is effected by contacting at least one terrestrial area with a herbicidally effective amount of any one of the compounds or compositions provided herein.

The term “terrestrial area” refers to the locus of the undesired vegetation, or the locus where the prevention of growth of undesired vegetation is sought, on land, in water, or in an artificial environment. A terrestrial area includes, without limitation, any agricultural areas, crop fields, gardens, greenhouses, wastelands, industrial or construction sites, roadsides, railways or railway embankments, and waterways, canals, reservoirs and ponds.

In some embodiments, compound is used as a pre-emergence herbicide, a post-emergence herbicide, a selective herbicide, a non-selective herbicide, an agricultural herbicide, a non-agricultural herbicide, an herbicide in integrated pest management, a gardening herbicide, an herbicide in clearing waste ground, an herbicide in clearing an industrial or a constructions site, or an herbicide in for clearing railways and railway embankments.

Unless specifically stated, as used herein, the term “about” is understood as within a range of normal tolerance in the art, for example within two standard deviations of the mean. In one embodiment, the term “about” means within 10% of the reported numerical value of the number with which it is being used, preferably within 5% of the reported numerical value. For example, the term “about” can be immediately understood as within 10%, 9%, 8%, 7%, 6%, 5%, 4%, 3%, 2%, 1%, 0.5%, 0.1%, 0.05%, or 0.01% of the stated value. In other embodiments, the term “about” can mean a higher tolerance of variation depending on for instance the experimental technique used. Said variations of a specified value are understood by the skilled person and are within the context of the present invention. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges, for example from 1-3, from 2-4, and from 3-5, as well as 1, 2, 3, 4, 5, or 6, individually. This same principle applies to ranges reciting only one numerical value as a minimum or a maximum. Unless otherwise clear from context, all numerical values provided herein are modified by the term “about”. Other similar terms, such as “substantially”, “generally”, “up to” and the like are to be construed as modifying a term or value such that it is not an absolute. Such terms will be defined by the circumstances and the terms that they modify as those terms are understood by those of skilled in the art. This includes, at very least, the degree of expected experimental error, technical error and instrumental error for a given experiment, technique or an instrument used to measure a value. Alternatively, the term “about” refers to ±10%.

The terms “comprises”, “comprising”, “includes”, “including”, “having” and their conjugates mean “including but not limited to”. The term “comprising”, used in the claims, is “open ended” and means the elements recited, or their equivalent in structure or function, plus any other element or elements which are not recited. It should not be interpreted as being restricted to the means listed thereafter; it does not exclude other elements or steps. It needs to be interpreted as specifying the presence of the stated features, integers, steps or components as referred to, but does not preclude the presence or addition of one or more other features, integers, steps or components, or groups thereof. Thus, for example, the scope of the expression “a composition comprising x and z” should not be limited to compositions consisting only of ingredients x and z. Also, the scope of the expression “a method comprising the steps x and z” should not be limited to methods consisting only of these steps.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

As used herein, the term “and/or” includes any and all combinations of one or more of the associated listed items. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

Various embodiments and aspects of the present invention as delineated hereinabove and as claimed in the claims section below find experimental and/or calculated support in the following examples.

### EXAMPLES

Reference is now made to the following examples, which together with the above descriptions illustrate some embodiments of the invention in a non-limiting fashion.

### Example 1

### Molecular Syntheses

Based on the elucidation and the validation of the pharmacophore and the identification of molecules in the library that were not known as herbicides or for their binding affinity to OASS, but exhibited the OASS-SAT binding inhibition, the present inventors have designed and prepared a group of exemplary novel compounds that exhibited OASS-SAT binding inhibition.

Exemplary compounds, according to embodiments of the present invention, falling under Scaffold 1 in Table 3, include compounds denoted PJl-106, PJL-126, PJL-107, PJL-131, PJL-110, PJL-116, and PJL-108 were prepared following Scheme 1 below:

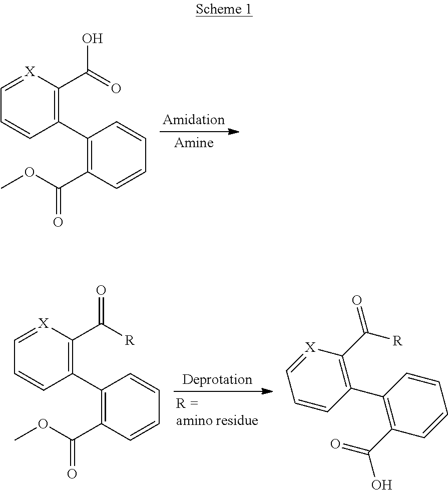

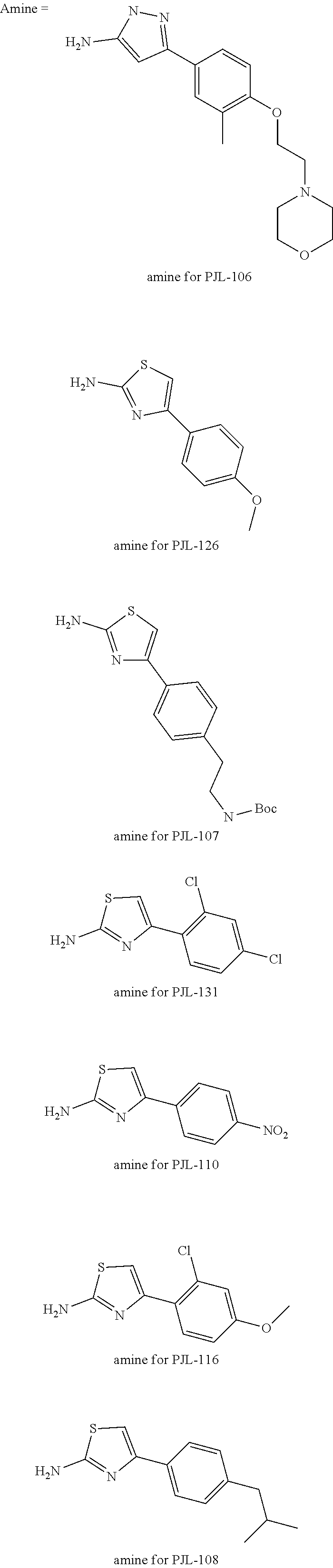

All novel compounds, according to embodiments of the present invention, were prepared similarly, following combinatorial synthesis guidelines known in the art.

Some exemplary compounds that include compounds denoted PJL-109, PJL-118 and PJL-119 were prepared following Scheme 2 below:

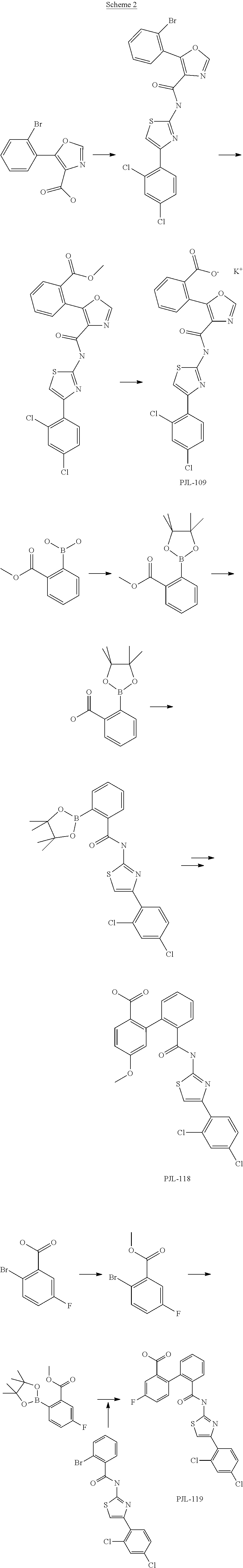

Some exemplary compounds were prepared following Scheme 3 below:

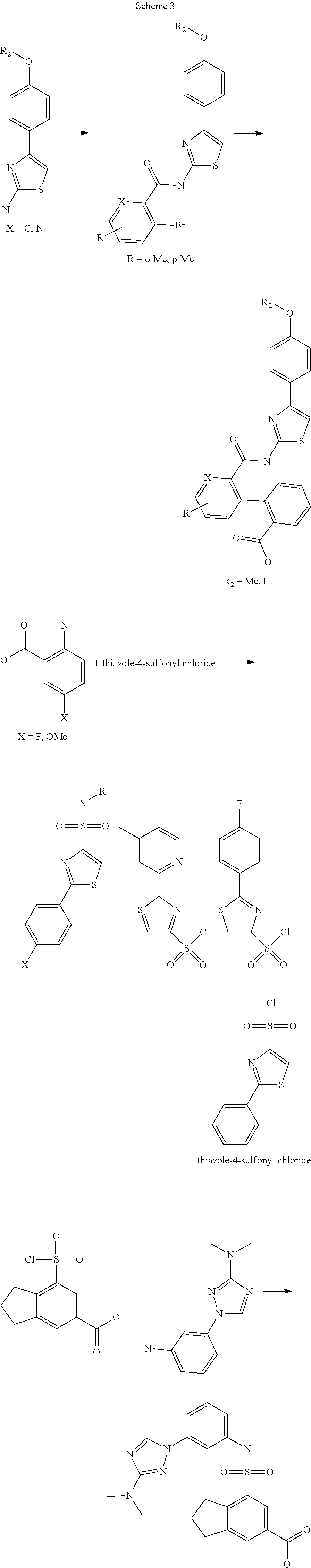

### Example 2

### Virtual and In Vitro Screening of Molecules

### Docking/Filtering and Selection of Virtual Hits:

Molecules of the filtered/profiled library that fit the physico-chemical and pharmacophore constraints were further selected for molecular docking onto the structure of Arabidopsis thaliana OASS (AtOASS; PDB ID: 2ISQ]. Docking was executed on an interface defined by radius of 11 Å from the center of SAT binding site (adjacent to Y905 ring) using GoldScore fitness function with no constraints. Ten different structural docking orientation and configuration were returned for each molecule. The best orientation of each molecule was selected considering the following criteria:
(i) the molecule shows carboxylate and hydrophobic groups at the location of AtOASS I907;
(ii) the molecule shows an aromatic ring in proximity to the AtOASS Y905 ring; and
(iii) orientation that made H-bond interaction with AtOASS S75 side-chain or backbone, and further preferably an interaction also with M125 backbone and Q147; and
(iv) orientation that shows a hydrophobic group in the hydrophobic pocket of AtOASS defined by P73, M125, 1129, M144, Q146, F148 and F230.

Each molecule ranked by two scores: Gold-based docking score and pose (orientation) score. Then top 40 percent ranked molecules were further filtered by the application of additional scoring functions PLP1, GoldPLP and ChemScore [The Cambridge Crystallographic Data Centre (CCDC) doi: 10.1002/prot.10465]. A consensus score based on the latter normalized scores (between 0-1), together with the previously defined docking score and pose score was applied to select the top ranked 30 percent molecules. These were clustered, and representative cluster centers were reviewed manually. Finally, 150 molecules were purchased for further in vitro and in planta evaluation.

### In Vitro Screening:

Selected molecules that exhibited virtual hits were purchased, and each molecule was dissolved in DMSO to a final concentration of 50 mM. Molecules were evaluated for their binding to OASS by two orthogonal methods, ITC and FP.

Two molecules showed direct binding to AtOASS by ITC and the ability to compete SAT-derived peptide by FP, PJS-120 and PJS-41.

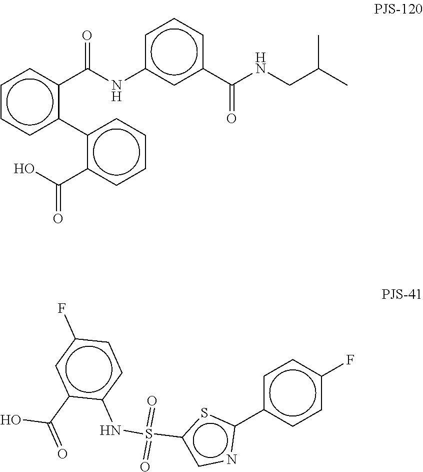

### Example 3

### Expression and Purification of OASS and Synthesis of SAT

The gene encoding Arabidopsis thaliana OASS amino acids was synthesized and subcloned into a pGex-6p-1 vector (GeneScript) containing and N-terminus GST tag followed by a Prescission Protease cleaving site. The plasmid was transformed into E. coli Rosetta (DE3) cells. Cells were cultured to OD=0.8 and protein expression was induced with the addition of 0.5 mM IPTG. The cells were cultured for an additional 16 hours at 25° C. and harvested by centrifugation at 4000 rpm for 20 minutes. Cell were lysed by sonication, and the lysate was centrifuged for 30 minutes at 20000 g and OASS was purified on a GST column. Following protein elution, the N-terminus GST-tag was cleaved by the application of Precision Protease followed by overnight dialysis and OASS was eluted with 20 mM reduced glutathione in PBS.

The synthesis of SAT fragment, corresponding to the 10 C-terminal residues of Arabidopsis thaliana SAT (AtSAT; C10; UniProtKB accession number Q42588) was synthesized by GeneScript with an N-terminus FITC tag and C-terminus acetylation.

### Example 4

### In Vitro and in Planta Evaluation of Herbicidal Activity

### Iso Thermal Calorimetry (ITC):

To evaluate the binding of the small molecules to the enzyme OASS, the purified protein was placed in a 6-8 MWCO dialysis bag in 30 mM phosphate buffer, 30 mM NaCl, 1 mM TCEP, and 5% dimethyl sulfoxide. Sequential buffer exchange dialyses were executed for 12 hours each at 4° C. until dilution of >10,000 was achieved. Before the execution of the ITC binding experiment, the OASS was placed in the ITC syringe at a concentration of 400 μM and each of the small molecules was dissolved in the same dialysis buffer to a final concentration of 40 μM in the ITC cell. ITC experiments were conducted by titrating the OASS to the compounds in the cell. The ITC binding constant was evaluated using the build-in analysis software.

### Fluorescence Polarization (FP):

Fluorescence polarization (FP) measurements were made on samples arrayed in 96-well plates by using BioTek H1 Synergy instrument equipped with FP module. A peptide derived from the sequence of SAT (sequence accession number Q42588) was synthesized with an N-terminus fluorescein isothiocyanate (FITC) tag. The peptide in a concentration of 200 nM was incubated with OASS at a concentration of 2.2 μM in each well. Variable concentrations of each molecule starting from 100 μM down to 0.048 μM were added to evaluate the ability of each molecule to compete off the FITC labeled peptide bound to OASS. Fluorescence experiments were executed with an Em/Exc wavelengths of 480/520 nm. All measurements were done in triplicate. Experimental polarization data from simple and competitive binding experiments were fitted using GraphPad Prism, with error bars representing SD.

### Root Elongation in Plates:

6-well plates were prepared in a biological hood with sterile half Murashige and Skoog mineral medium (MS; Duchefa, Haarlem, Netherlands) containing 1% sucrose (w/v), pH 5.8. Tested molecules were dissolved in 50 mM DMSO and diluted to various concentrations 1,000-fold greater than the final desired concentration the MS medium maintaining a final concentration of 0.1% DMSO. MS medium with 0.1% DMSO was used as a negative control. After 7 days the plates were scanned and the root length was calculated using the imageJ software.

### L-Cysteine Recovery Assay:

For the L-cysteine recovery root elongation assay, 6-well plates were prepared in a biological hood with sterile half Murashige and Skoog mineral medium (MS; Duchefa, Haarlem, Netherlands) containing 1% sucrose (w/v), pH 5.8. A stock of 10 mM L-cysteine in DDW was prepared and diluted to a final concentration of 0.03 mM into the MS medium in the presence of 25 to 1.56 μM of PJL 109. The MS medium maintained a final concentration of 0.1% DMSO. MS medium with 0.1% DMSO and 0.03 mM L-cysteine was used as a negative control. After 7 days the plates were scanned and the root length was calculated using the imageJ software.

Preparation of Arabidopsis thaliana Seeds:

Arabidopsis thaliana seeds were washed with 70% ethanol for 1 minute and then with sterile water. Further sterilization was done by washing the seeds with 50% hypochlorite and 0.2% Triton x-100 for 10 minutes followed by five cycles of washing with sterile water. Six seeds were placed in each well, and the plates were covered and sealed with parafilm. Plates were placed vertically at 4° C. for two days and then transferred to a growth chamber for seven days (22-24° C.) at day/light cycles of 16/8 hours.

### Pre-Emergence Assay in Pots:

7×7 cm pots were prepared with 20% soil and 80% sand. Trifolium alexandrium (16 seeds), Lolium rigidum (25 seeds), Solanum nigrum (25 seeds), Amaranthus palmeri (40 seeds) or Sinapis arvensis (12 seeds) were sowed and 2 cm of strained sand was used to cover the seeds. The pots were placed in the growth room under a lighting regime of 16 hours of light and 8 hours in the dark at 23° C. The day after (except for the Solanum and Amaranthus 4-5 days after), the pots were sprayed in a chemical hood at a 45° angle with 2 ml spray solution containing 1:10 acetone or phosphate buffer dissolved active ingredient in tap water to a final concentration of 5 mM. Every 2-3 days the pots were irrigated with tap water.

### Example 5

### Results

### In Vitro Binding Results (FP and ITC):

The designed de-novo herbicidal compounds were synthesized and evaluated for their in vitro and in planta activity. After dissolving the molecules to a stock of 50 mM in DMSO, binding and competition with the SAT-derived peptides were evaluated using ITC and FP. ITC measures the direct heat change occurring due to the interaction of a pair of molecules. ITC has certain advantages, such as the capability to detect a relatively broad range of binding affinities, being a label-free method, and providing a wide set of interaction parameters, such as affinity, stoichiometry, enthalpy, and entropy of binding. To evaluate the ability of the molecules to bind AtOASS, the protein was placed in the ITC syringe and was titrated with the molecules that were placed in the ITC cell. The direct dissociation binding constant (Kd) was then extracted from the binding curve using a single binding model. Two consecutive experiments were combined using the built in ITC module to enable better quantification. A low Kd value corresponds to a stronger binding affinity.

Molecules were also tested for their ability to compete with the OASS binding to the SAT-derived peptide via a fluorescence polarization (FP) competition assay. The N-terminus labeled peptide was incubated with the AtOASS in each of the wells in a 96-well plate. Various concentrations of the molecules were added and the FP values were recorded. High FP values correspond to the peptide-bound state (low molecule concentrations) and low FP values correspond to the peptide-free state (low FP values). To extract the IC50, the FP data were fitted to a single site inhibition model in graphpad prism. The corresponding IC50 values were extracted. Low IC50 values correspond to molecule that better competes with the peptide binding to AtOASS.

### In Planta Root Elongation Assay:

Following the in vitro evaluation, selected molecules were tested for their ability to inhibit A. thaliana root elongation on agar plates. Seeds were plated on medium containing the molecules dissolved in the indicated concentrations and root length was measured after 6 or 7 days when the root in the non-treated plate reached the end of the well.

### Pre-Emergence Assay:

Pre-emergence herbicidal activity was determined by monitoring plant growth in pots. Molecules were dissolved at the indicated concentrations below and volume of 2 ml was sprayed onto the soil, and plant growth was monitored for 21 days.

Table 4 above presents the activity assays results obtained for a selection of herbicidal compounds, according to some embodiments of the present invention.

The results of the experimental A. thaliana growth in the presence of various concentrations of the exemplary compound PJL-109, with 0.03 mM, and no external addition of cysteine, show that the addition of cysteine to the agar plates partially reverses the germination inhibition effect of the tested molecule in a dose-response manner. The IC50 value in the presence of 0 or 0.03 mM L-cysteine was 1.7 μM and 4.9 μM respectively indicating that the molecule binds to the target enzyme, OASS, and inhibits the biosynthesis of cysteine.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, contents of publicly accessible databases, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A compound of Formula (I): 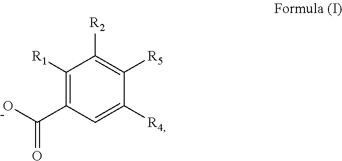
wherein R₁ is selected from hydrogen atom, a radical of Formula (A), a radical of Formula (A2), a radical of Formula (B), and a radical of Formula (C): 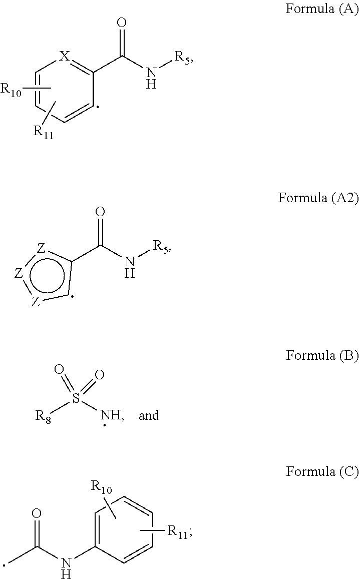
X and Z are each independently carbon, oxygen, or nitrogen,
R₂ is selected from hydrogen atom, radical of formula (D), and the following radical of Formula (E): 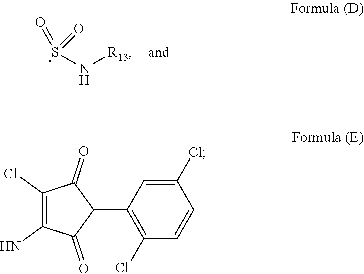
R₃ and R₄ are independently selected from hydrogen, (C₁-C₃)-alkyl, (C₁-C₃)-haloalkyl, amino, cyano, hydroxy, (C₁-C₃)-alkoxy, halogen, and nitro group; or
R₃ and R₄ are linked via (C₁-C₃)-alkyl to form a ring;
R₅ is selected from any one of the following radicals: 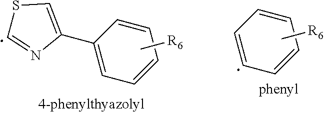
R₆ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, nitro group, and any one of the following radicals: 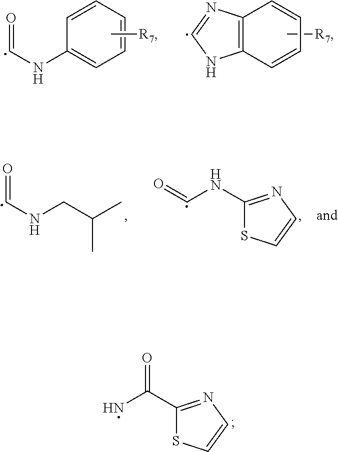
R₇ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, and nitro group;
R₈ is selected from any one of the following radicals: 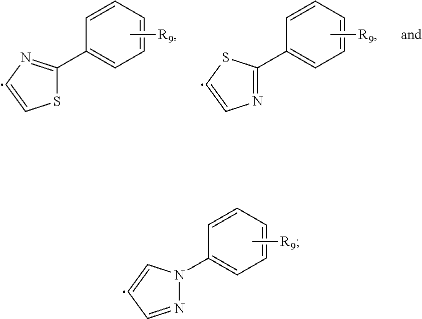
R₉ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, and nitro group;
R₁₀ and R₁₁ are attached to any available phenyl ring carbon atom and independently selected from hydrogen, (C₁-C₃)-alkyl, (C₁-C₃)-haloalkyl, (C₁-C₃)-alkoxy, halogen, amino, cyano, hydroxy, nitro group, and any one of the following radicals: 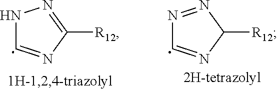
R₁₂ is selected from hydrogen, (C₁-C₃)-alkyl, and (C₁-C₃)-haloalkyl;
R₁₃ selected from any one of the following radicals: 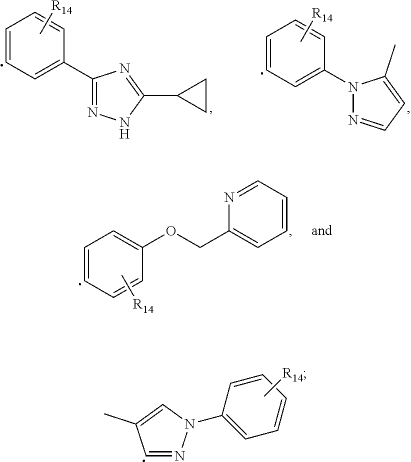
R₁₄ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, nitro group;
(i) provided that when R₁ is selected from the group consisting of said radical of Formula (A), said radical of Formula (A2), and said radical of Formula (C), then
R₂, R₃ and R₄ are hydrogens;
(ii) provided that when R₁ is said radical of Formula (B), then
R₂ is hydrogen, and
R₃ and R₄ are independently selected from hydrogen, (C₁-C₃)-alkyl, (C₁-C₃)-haloalkyl, amino, cyano, hydroxy, (C₁-C₃)-alkoxy, trifluoromethyl, halogen, and nitro group;
(iii) provided that when R₁ is said radical of Formula (C), then
R₁₀ and R₁₁ are not (C₁-C₃)-haloalkyl, (C₁-C₃)-alkoxy, halogen, amino, or cyano;
(iv) provided that when R₅ is phenyl, then
R₆ is not hydrogen, (C₁-C₃)-alkyl, or nitro group;
(v) provided that when R₂ is said radical of formula (D), then
R₁ is hydrogen, and
R₃ and R₄ are linked via (C₁-C₃)-alkyl to form a ring; and
(vi) provided that when R₂ is the radical of formula (E), then
R₁, R₃ and R₄ are hydrogens.

2. The compound of claim 1 having Formula (IA): 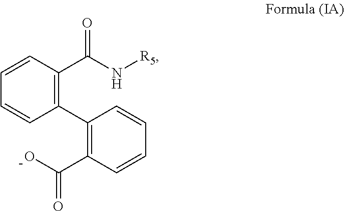
wherein R₅ is the 4-phenylthyazolyl of the following formula: 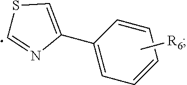
R₆ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, and nitro group.

3. (canceled)

4. The compound of claim 1 having Formula (IA): 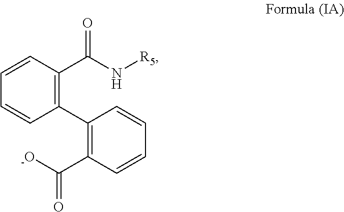
wherein R₅ is the phenyl of the following formula: 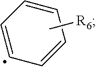
R₆ is attached to any available phenyl ring carbon atom and selected from halogen, hydroxy, (C₁-C₃)-haloalkyl, (C₁-C₃)-alkoxy, amino, cyano, and any one of the following radicals: 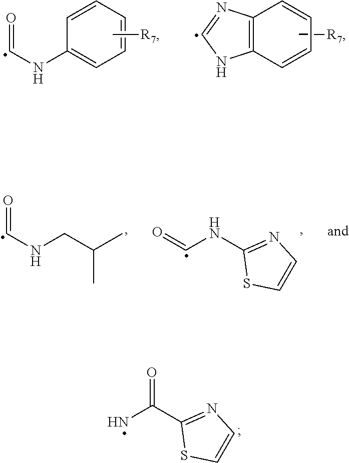
R₇ is attached to any available phenyl ring carbon atom and independently selected from hydrogen, (C₁-C₃)-alkyl, (C₁-C₃)-haloalkyl, (C₁-C₃)-alkoxy, halogen, amino, cyano, hydroxy, and nitro group.

5-8. (canceled)

9. The compound of claim 1 having Formula (IB): 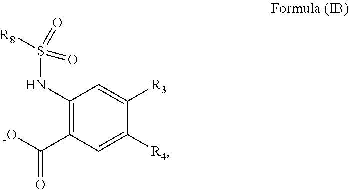
wherein R₃ and R₄ are independently selected from hydrogen, halo, hydroxy, amino, cyano, (C₁-C₃)-alkyl, (C₁-C₃)-haloalkyl, (C₁-C₃)-alkoxy, and nitro group;
R₈ is selected from any one of the following radicals: 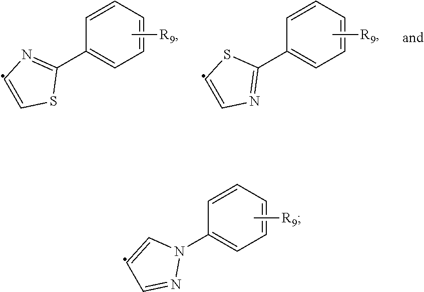
R₉ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, and nitro group.

10-11. (canceled)

12. The compound of claim 1 having Formula (IC): 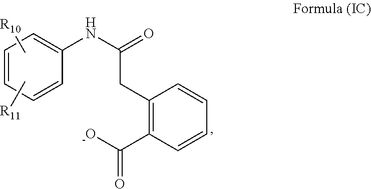
wherein R₁₀ and R₁₁ are attached to any available phenyl ring carbon atom and independently selected from hydrogen, (C₁-C₃)-alkyl, hydroxy, nitro group, and any one of the following radicals: 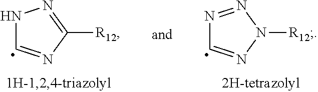
R₁₂ is selected from hydrogen, (C₁-C₃)-alkyl, and (C₁-C₃)-haloalkyl.

13-15. (canceled)

16. The compound of claim 1 having Formula (ID): 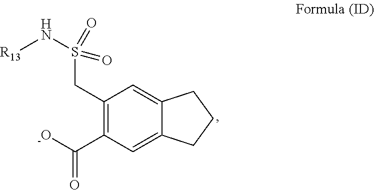
wherein R₁₃ selected from any one of the following radicals: 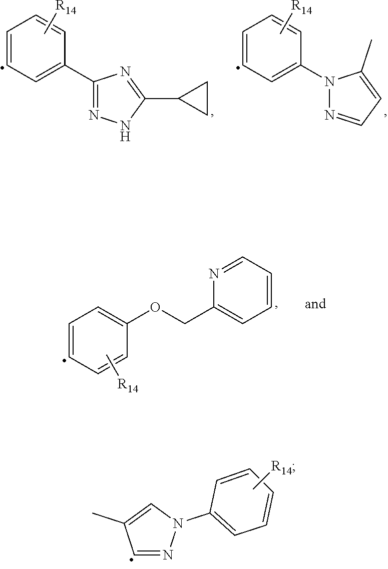
R₁₄ is attached to any available phenyl ring carbon atom and selected from hydrogen, halogen, hydroxy, (C₁-C₃)-alkyl, (C₁-C₃)-haloalkyl, (C₁-C₃)-alkoxy, amino, cyano, nitro group.

17-18. (canceled)

19. A compound selected from the group consisting of:

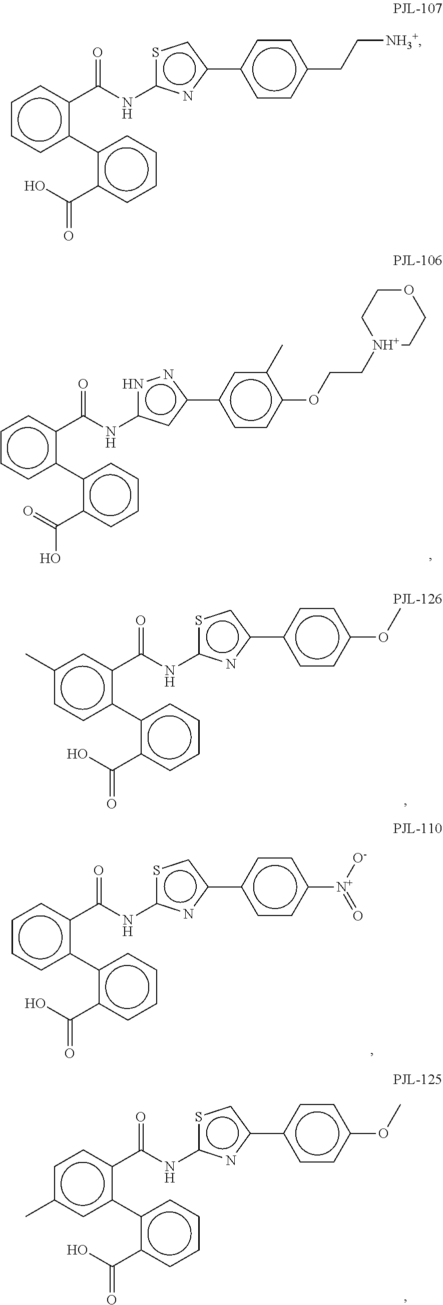

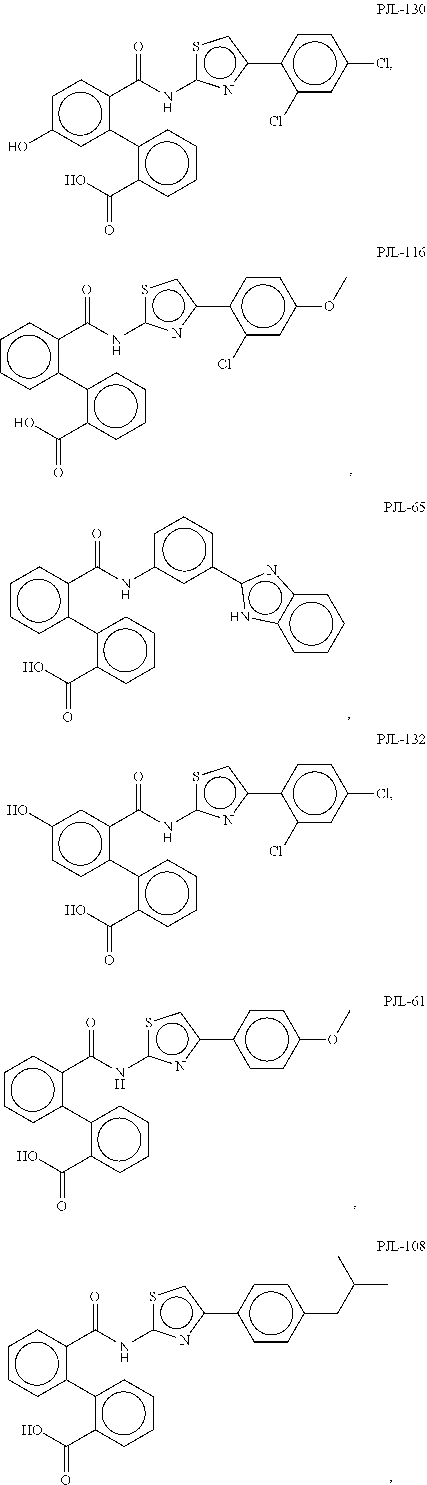

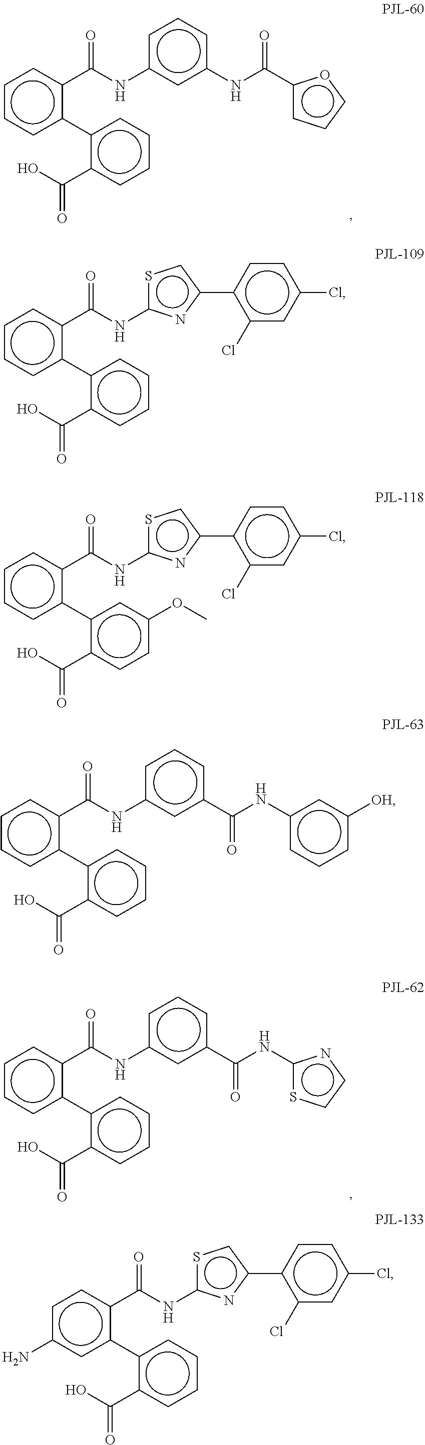

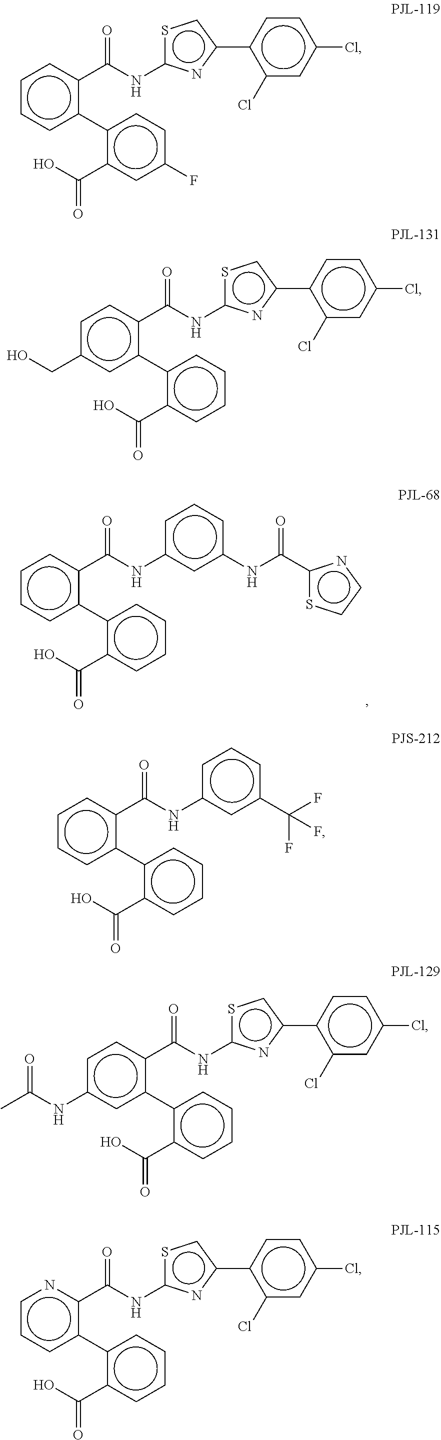

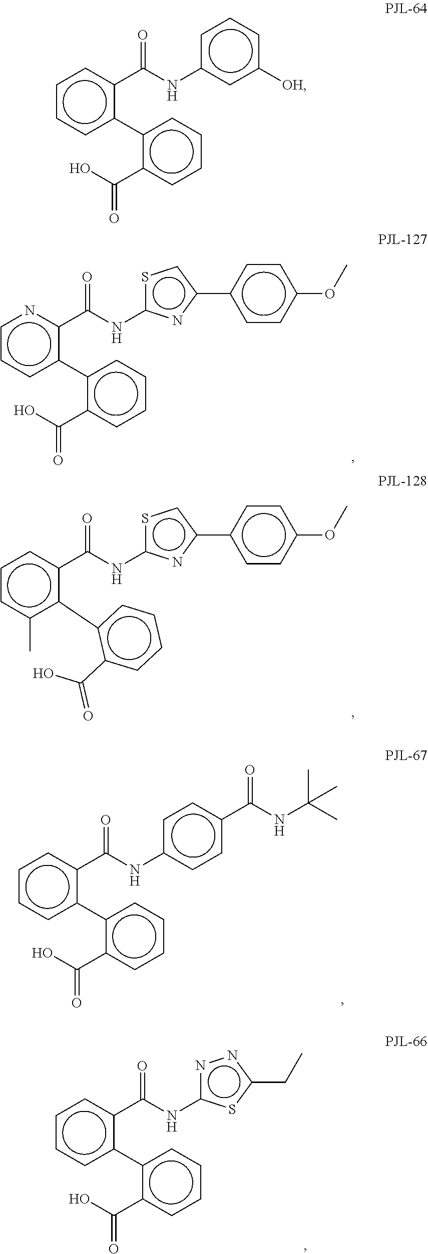

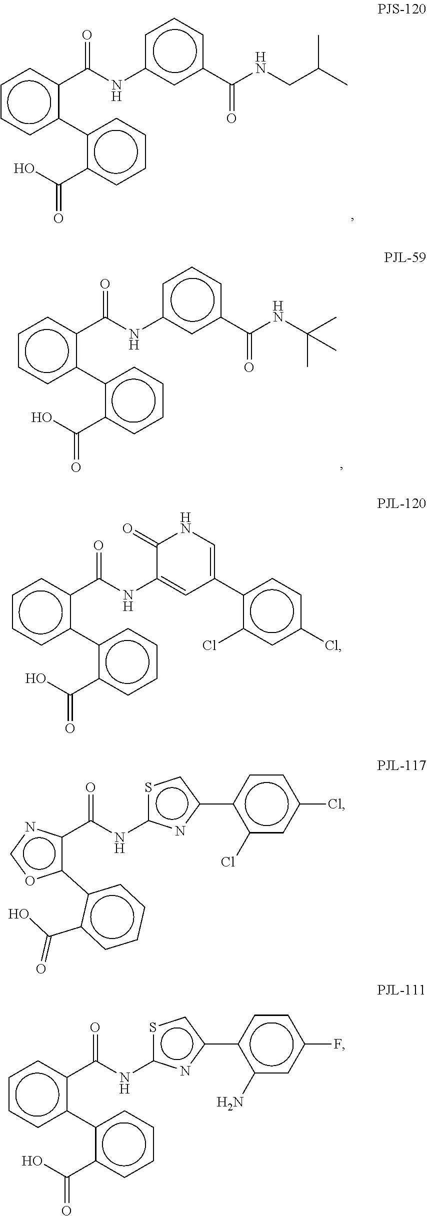

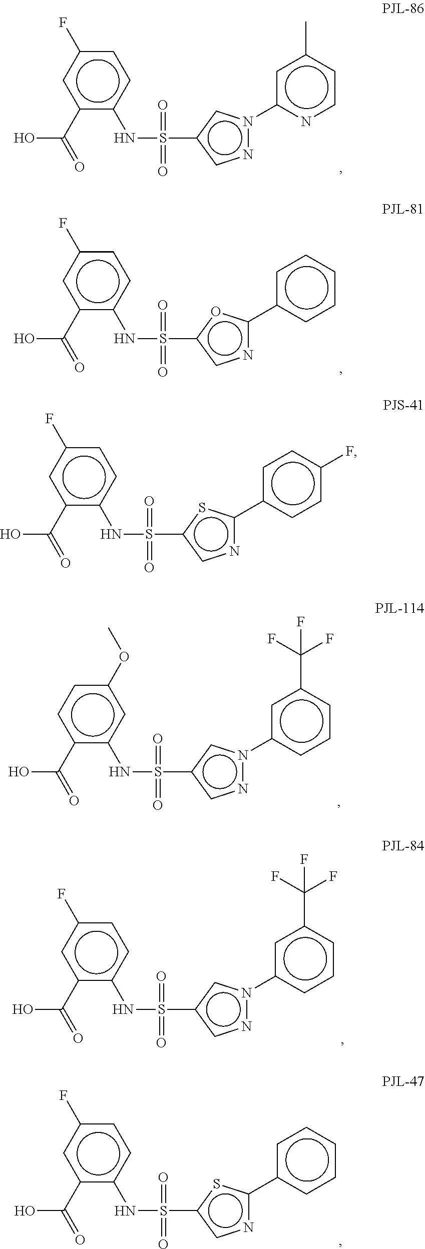

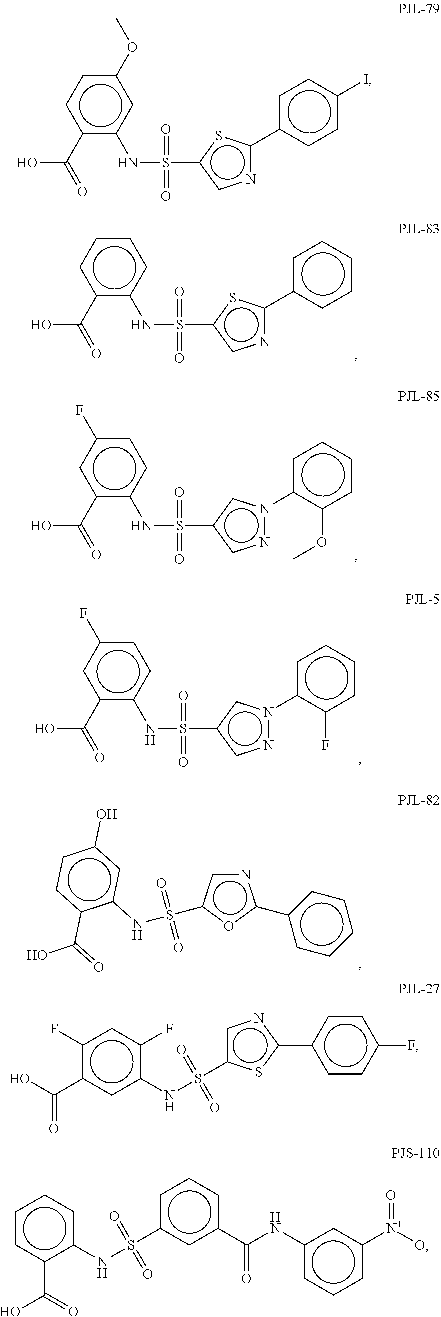

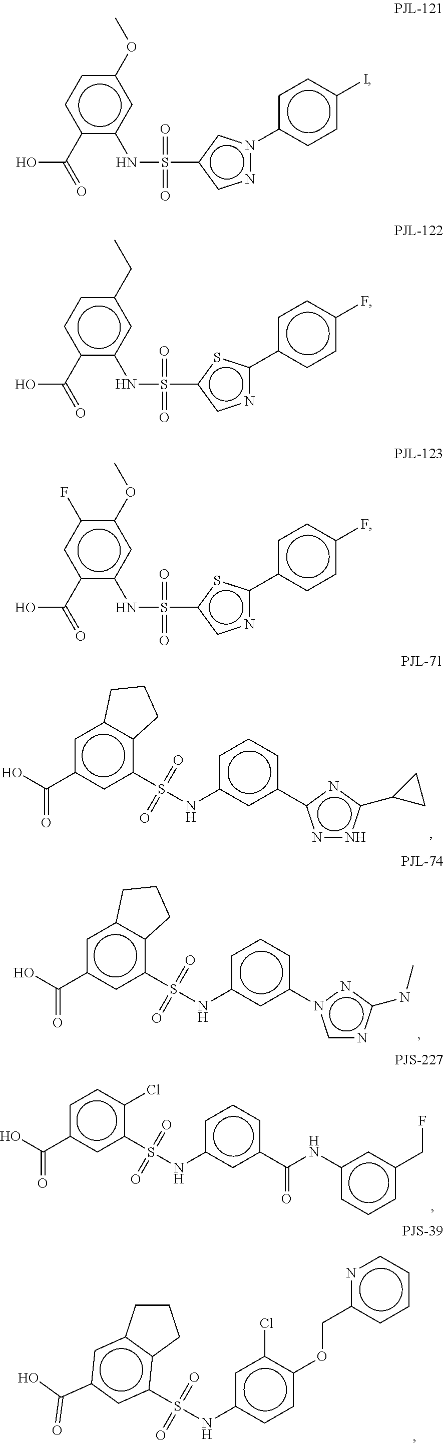

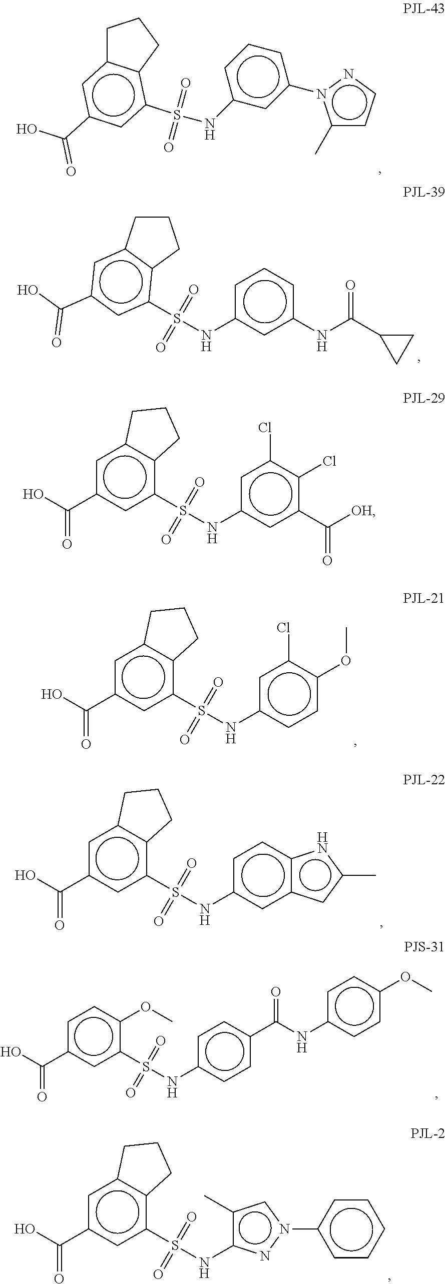

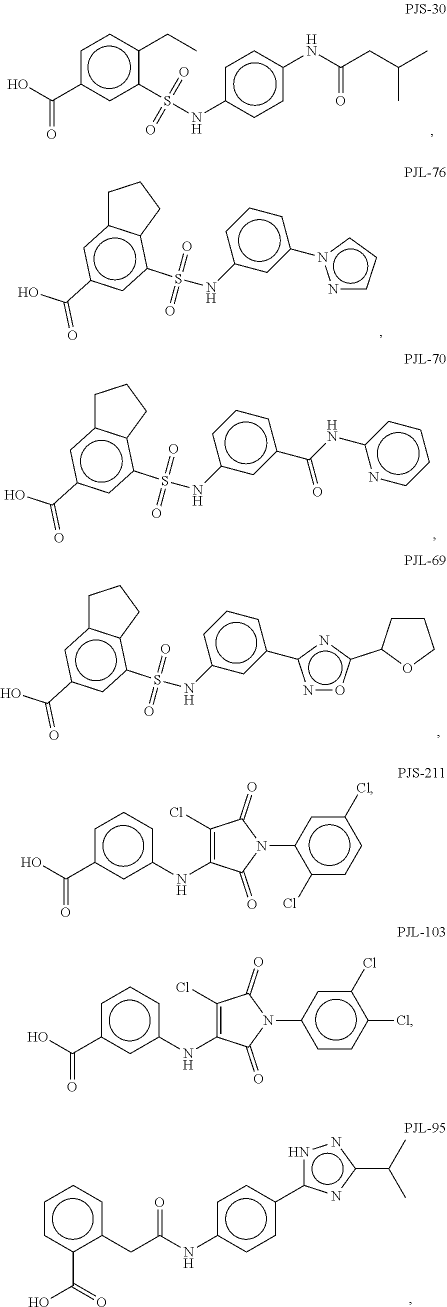

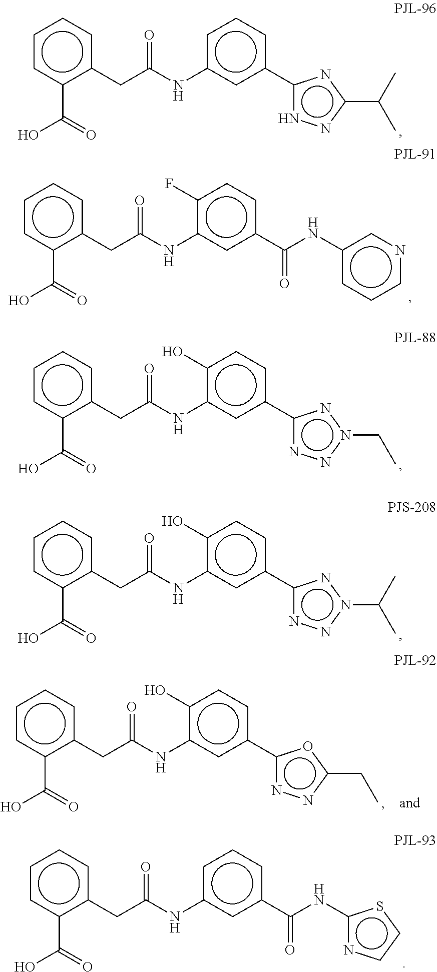

20. A compound inhibiting the binding of O-acetyl serine sulfhydrylase (OASS) to serine acetyltransferase (SAT), comprising:
15-40 non-hydrogen atoms;
2-7H-bond acceptors;
0-3H-bond donors;
1-2 functional groups capable of exhibiting a negative charge;
0-1 functional groups capable of exhibiting a positive charge;
2-9 rotatable bonds;
1-2 hydrophobic functional groups;
0-4 non-aromatic rings; and
2-5 aromatic rings;
said atoms, and/or said functional groups, and/or said bonds, and/or said rings, form at least three structural determinants positioned on an arbitrary 3D cartesian coordinates system (in angstroms; Å), selected from the group consisting of:
a first hydrophobic functional group at position 78.51, 47.01, −12.92 within a radius of 1.6 (HD1);
a second hydrophobic functional group at position 78.26, 49.86, −6.08 within a radius of 1.6 (HD2);
a functional group capable of exhibiting a negative charge at position 75.93, 45.12, −12.53 within a radius of 1.7 (NC1);
an aromatic ring at position 77.12, 51.92, −10.61 within a radius of 1.6 (AR1), said aromatic ring is having a ring projection at position 80.05, 51.32, −10.93 within a radius of 2.2 (A1_{P}); and
a H-bond acceptor at position 74.18, 50.71, −13.04 within a radius of 1.6 (HA1), said H-bond acceptor is having a ring projection at position 71.80, 49.22, −11.99 within a radius of 2.2 (HA1_{P}),
and characterized by:
a Log P that ranges 0.5-5.5;
a molecular mass that ranges 300-600 g/mol;
wherein the compound exhibiting at least one property selected from the group consisting of:
k_{d} lower that 100 m as determined in vitro by an iso thermal calorimetry (ITC) assay;
IC50 lower that 200 m as determined in-vitro by a fluorescence polarization (FP) assay;
at least 10% inhibition as determined in-vitro by an FP assay; and
at least 10% inhibition as determined by an in-planta root elongation assay.

21. The compound of claim 20, wherein said structural determinants lack atoms in at least one position selected from the group consisting of Table 2B.

22. The compound of claim 20, wherein said structural determinants lack atoms in positions listed in Table 2B.

23. The compound of claim 20, wherein each of said structural determinants exhibit positioning and orientation so as to interact with at least one residue in OASS, wherein:
HD1 interacts with F148, G181 and/or A228;
AR1 interacts with F148 and/or M125;
NC1, interacts with K46, T78 and/or Q147;
HA1 interacts with S75, T74 and/or Q147; and
HD2 interacts with 1129, F148 and/or F230.

24-25. (canceled)

26. The compound of claim 20, characterized by a general formula selected from the group consisting of: 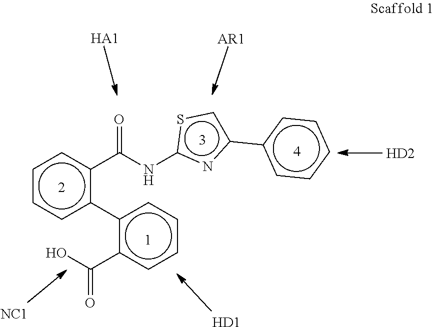
wherein rings 2 and 4 are each independently a substituted or unsubstituted 5 or 6 membered aryl or heteroaryl, 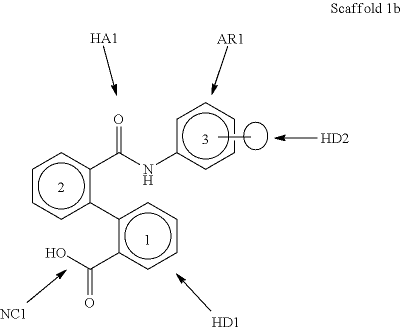
wherein ring 3 is a substituted or unsubstituted 5 or 6 membered aryl or heteroaryl, 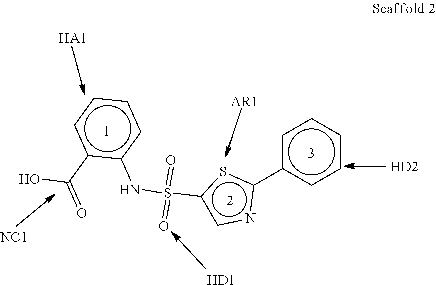
wherein ring 3 is a substituted or unsubstituted 5 or 6 membered aryl or heteroaryl, 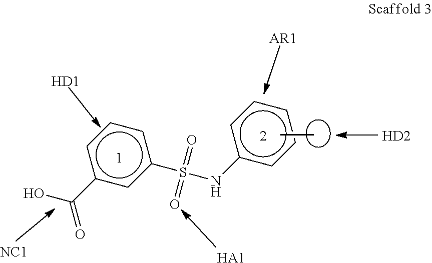
wherein ring 2 is a substituted or unsubstituted 5 or 6 membered aryl or heteroaryl, 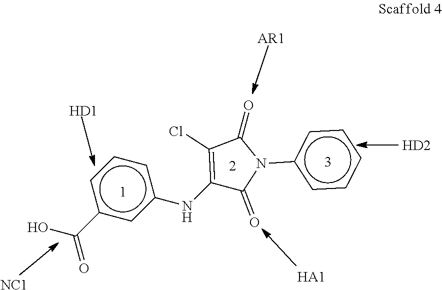
wherein ring 3 is a substituted or unsubstituted 5 or 6 membered aryl or heteroaryl, and 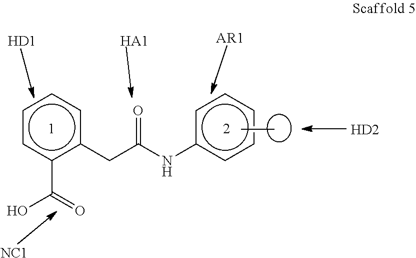
wherein ring 2 is a substituted or unsubstituted 5 or 6 membered aryl or heteroaryl.

27. The compound of claim 26, wherein:
Scaffold 1 is represented by the compounds selected from the group consisting of PJL-107, PJL-106, PJL-126, PJL-110, PJL-125, PJL-130, PJL-116, PJL-65, PJL-132, PJL-61, PJL-108, PJL-60, PJL-109, PJL-118, PJL-133, PJL-119, PJL-131, PJS-212, PJL-129, PJL-115, PJL-64, PJL-127, PJL-128, PJL-67, PJL-66, PJL-59, PJL-120, PJL-117, and PJL-111;
Scaffold 1b is represented by the compounds selected from the group consisting of PJL-59, PJL-60, PJL-62, PJL-63, PJL-68, and PJS-120;
Scaffold 2 is represented by the compounds selected from the group consisting of PJL-86, PJL-81, PJS-41, PJL-114, PJL-84, PJL-47, PJL-79, PJL-83, PJL-85, PJL-5, PJL-82, PJL-27, PJS-110, PJL-121, PJL-122, and PJL-123;
Scaffold 3 is represented by the compounds selected from the group consisting of PJL-27, PJL-71, PJL-74, PJS-227, PJS-39, PJL-43, PJL-39, PJL-29, PJL-21, PJL-22, PJS-31, PJL-2, PJS-30, PJL-76, PJL-70, and PJL-69;
Scaffold 4 is represented by the compounds selected from the group consisting of PJS-211 and PJL-103; and
Scaffold 5 is represented by the compounds selected from the group consisting of PJL-95, PJL-96, PJL-91, PJL-88, PJS-208, PJL-92, and PJL-93.

28. The compound of claim 1, capable of entering a plant cell.

29. The compound of claim 1, capable of entering a plant plastid/chloroplast.

30. An herbicidal composition comprising, as an active ingredient, the compound of claim 1, and an agronomically acceptable carrier and optionally at least one adjuvant, said carrier or said adjuvant is for allowing the compound to be used as herbicide.

31-38. (canceled)

39. A method of inhibiting the binding of SAT to OASS, comprising contacting OASS with a compound, said compound comprises functional groups at position and orientation so as to interact with at least three groups of amino acid residues of OASS, said groups of amino acid residues are selected from the group consisting of:
F148, G181 and/or A228;
F148 and/or M125;
K46, T78 and/or Q147;
S75, T74 and/or Q147; and
I129, F148 and/or F230.

40-41. (canceled)

42. A method of inhibiting the post-emergence growth of a plant, comprising contacting the plant with a herbicidally effective amount of the compound of claim 1.

43. A method of inhibiting the pre-emergence growth of a plant, comprising contacting at least one terrestrial area with a herbicidally effective amount of the compound of claim 1.

44. A method of controlling undesired vegetation growth, comprising applying to at least one terrestrial area of said undesired vegetation a herbicidally effective amount of the compound of claim 1.

45-46. (canceled)
